SEQUENCE LISTING (23 sequences)

SEQ ID NO: 1 - RNA, 21 nt, Artificial Sequence
  single-stranded RNA (ssRNA) 2′OMe-uridine homopolymer 21 mer, polyUmod21, (mU)-21, polyU21, polyU-21-Umod
uuuuuuuuuuuuuuuuuuuuu

SEQ ID NO: 2 - RNA, 15 nt, Artificial Sequence
  single-stranded RNA (ssRNA) 2′OMe-uridine homopolymer 15 mer, polyUmod15, (mU)-15, polyU15, polyU-15-Umod
uuuuuuuuuuuuuuu

SEQ ID NO: 3 - RNA, 10 nt, Artificial Sequence
  single-stranded RNA (ssRNA) 2′OMe-uridine homopolymer 10 mer, polyUmod10, (mU)-10, polyU10, polyU-10-Umod
uuuuuuuuuu

SEQ ID NO: 4 - RNA, 21 nt, Artificial Sequence
  firefly luciferase (Luc) sense strand single-stranded RNA (ssRNA) having 2′-OMe modifications at u residues (Luc UmodS) (Luc-mU)
gauuauguccgguuauguauu

SEQ ID NO: 5 - RNA, 21 nt, Artificial Sequence
  Escherichia coli beta-galactosidase (beta-gal) sense strand single-stranded RNA (ssRNA)
cuacacaaaucagcgauuuuu

SEQ ID NO: 6 - RNA, 21 nt, Artificial Sequence
  cystic fibrosis transmembrane conductance regulator ATP-binding cassette sub-family C, member 7/Aequorea victoria enhanced green fluorescent protein (GFP) fusion protein (CFTR/EGFP) sense strand single-stranded RNA (ssRNA) (GFP-S)
ggcuacguccaggagcgcauu

SEQ ID NO: 7 - RNA, 20 nt, Artificial Sequence
  GU rich RNA40 single-stranded RNA (ssRNA)
gcccgucuguugugugacuc

SEQ ID NO: 8 - RNA, 21 nt, Artificial Sequence
  influenza A virus nucleocapsid protein (NP) NP1496 sense strand single-stranded RNA (ssRNA)
ggaucuuauuucuucggaguu

SEQ ID NO: 9 - RNA, 21 nt, Artificial Sequence
  influenza A virus nucleocapsid protein (NP) NP1496 sense strand single-stranded RNA (ssRNA) having 2′-OMe modifications at u residues (NP1496 UmodS) (NP-mU)
ggaucuuauuucuucggaguu

SEQ ID NO: 10 - RNA, 21 nt, Artificial Sequence
  firefly luciferase (Luc) sense strand single-stranded RNA (ssRNA)
gauuauguccgguuauguauu

SEQ ID NO: 11 - RNA, 21 nt, Artificial Sequence
  Escherichia coli beta-galactosidase (beta-gal) sense strand single-stranded RNA (ssRNA) having 2′-OMe modifications at u residues (beta-gal UmodS), betagal-mU
cuacacaaaucagcgauuuuu

SEQ ID NO: 12 - RNA, 23 nt, Artificial Sequence
  human apolipoprotein B (ApoB) antisense strand (AS) single-stranded RNA (ssRNA) (ApoB-AS)
auugguauucagugugaugacac

SEQ ID NO: 13 - RNA, 21 nt, Artificial Sequence
  Escherichia coli beta-galactosidase (beta-gal) antisense strand (AS) single-stranded RNA (ssRNA) (beta-gal AS)
aaaaaucgcugauuuguguag

SEQ ID NO: 14 - RNA, 21 nt, Artificial Sequence
  human apolipoprotein B (ApoB) sense strand single-stranded RNA (ssRNA) (ApoB-S)
gucaucacacugaauaccaau

SEQ ID NO: 15 - RNA, 21 nt, Artificial Sequence
  Escherichia coli beta-galactosidase (beta-gal) antisense strand (AS) single-stranded RNA (ssRNA) (betagal-AS)
aaaucgcugauuuguguaguu

SEQ ID NO: 16 - RNA, 21 nt, Artificial Sequence
  Escherichia coli beta-galactosidase (beta-gal) single-stranded RNA (ssRNA) having 2′-OMe modifications at c residues (betagal-mC)
cuacacaaaucagcgauuuuu

SEQ ID NO: 17 - RNA, 21 nt, Artificial Sequence
  Escherichia coli beta-galactosidase (beta-gal) single-stranded RNA (ssRNA) having 2′-OMe modifications at g residues (betagal-mG)
cuacacaaaucagcgauuuuu

SEQ ID NO: 18 - RNA, 21 nt, Artificial Sequence
  Escherichia coli beta-galactosidase (beta-gal) single-stranded RNA (ssRNA) having 2′-OMe modifications at a residues (betagal-mA)
cuacacaaaucagcgauuuuu

SEQ ID NO: 19 - RNA, 21 nt, Artificial Sequence
  influenza A virus nucleocapsid protein (NP) single-stranded RNA (ssRNA) having 2′-OMe modifications at c residues (NP-mC)
ggaucuuauuucuucggaguu

SEQ ID NO: 20 - DNA, 20 bp, Artificial Sequence
  synthetic DNA oligonucleotide ODN 2216 (PO)
gggggacgatcgtcgggggg

SEQ ID NO: 21 - DNA, 25 bp, Artificial Sequence
  synthetic DNA oligonucleotide ODN M362 (PO)
tcgtcgtcgttcgaacgacgttgat

SEQ ID NO: 22 - DNA, 16 bp, Artificial Sequence
  synthetic DNA oligonucleotide ODN 6295 (PO)
taacgttgaggggcat

SEQ ID NO: 23 - DNA, 20 bp, Artificial Sequence
  synthetic DNA oligonucleotide ODN 1826 (PO)
tccatgacgttcctgacgtt

(19) United States
(12) Patent Application Publication
(10) Pub. No.: US 2008/0171716 A1
(43) Pub. Date: Jul. 17, 2008

(54) NUCLEIC ACID MODULATION OF TOLL-LIKE RECEPTOR-MEDIATED IMMUNE STIMULATION

(75) Inventors: Ian MacLachlan, Mission (CA); Marjorie Robbins, Vancouver (CA); Adam Judge, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Protiva Biotherapeutics, Inc., Burnaby (CA)

(21) Appl. No.: 11/839,065

(22) Filed: Aug. 15, 2007

Related U.S. Application Data
(60) Provisional application No. 60/838,344, filed on Aug. 16, 2006.
(60) Provisional application No. 60/933,839, filed on Jun. 7, 2007.

(51) Int. Cl. A61K 31/70; C07H 21/00; A61P 25/00

(52) U.S. Cl. 514/44; 536/24.5

(57) ABSTRACT

The present invention provides methods of modulating the activation of certain Toll-like receptors (TLRs) such as TLR7/8 using chemically modified nucleic acid molecules. The present invention also provides methods of using such modified nucleic acid molecules to treat diseases or disorders associated with TLR7/8 activation such as systemic lupus erythematosus. The present invention further provides compositions comprising a combination of modified nucleic acid molecules and nucleic acid molecules that silence expression of one or more target sequences. Methods of using such compositions to reduce or abolish target gene expression without inducing cytokine production are also provided.

### CROSS-REFERENCE TO RELATED APPLICATIONS

This application is related to U.S. Provisional Application No. 60/838,344, filed Aug. 16, 2006, and U.S. Provisional Application No. 60/933,839, filed Jun. 7, 2007, the disclosures of which are herein incorporated by reference in their entirety for all purposes.

### BACKGROUND OF THE INVENTION

After the discovery of the protein Toll as a signaling receptor for immunity in Drosophila melanogaster, several homologous Toll-like receptors (TLRs) have been identified in mammals. TLRs are key receptors of the innate immune system and recognize a diverse range of conserved microbial molecules (Janeway et al., Annu. Rev. Immunol., 20:197-216 (2002); Akira et al., Nat. Rev. Immunol., 4:499-511 (2004)). Four out of the ten TLRs identified in humans recognize nucleic acids, demonstrating the fundamental importance of microbial DNA and RNA in triggering innate responses to pathogenic microorganisms (Hemmi et al., Nature, 408:740-745 (2000); Alexopoulou et al., Nature, 413:732-738 (2001); Diebold et al., Science, 303:1529-1531 (2004); Heil et al., Science, 303:1526-1529 (2004)).

Although TLR-mediated activation can initiate rapid and effective control of infection, the consequences to the host can be chronic or acute inflammation. For example, the roles of TLR2 and TLR4 have been demonstrated in microbial sepsis (Beutler, Nature, 430:257-263 (2004)). TLR3 has been shown to be required for the central nervous system inflammation that leads to the disruption of the blood-brain barrier during West Nile virus infection in mice (Wang et al., Nat. Med., 10:1366-1373 (2004)). This demonstrates that the nucleic acid component of a pathogen such as an RNA virus can trigger inflammation destructive to host tissues. In addition, TLR activation by endogenous ligands has been reported in some types of sterile inflammation (Andreakos et al., Immunol. Rev., 202:250-265 (2004); Rifkin et al., Immunol. Rev., 204:27-42 (2005)). For example, TLR2 and TLR4 respond to endogenous heat-shock proteins, TLR4 to extracellular matrix fragments, fibrinogen, and β-defensin (Smiley et al., J. Immunol., 167:2887-2894 (2001); Biragyn et al., Science, 298:1025-1029 (2002)), and TLR3 to mRNA (Kariko et al., J. Biol. Chem., 279:12542-12550 (2004)), all of which may be present at elevated levels at sites of tissue injury and inflammation. Similarly, DNA-anti-DNA IgG immune complexes (ICs) have been shown to stimulate autoantibody production in mice by a process involving TLR9 (Leadbetter et al., Nature, 416:603-607 (2002)).

By analogy to the adaptive immune system, the innate immune system requires mechanisms for self-nonself discrimination. Discrimination between nucleic acids of mammalian versus microbial origin by TLRs is particularly difficult, and the expression of the DNA- and RNA-specific TLRs in endosomal vesicles, but not on the cell surface, may represent one mechanism for restricting the response to nucleic acids from invading microorganisms. However, the failure of TLRs to discriminate between self and nonself nucleic acids may contribute to inflammation and autoimmunity.

For example, immune complexes of autoantibodies to chromatin and RNA protein particles (snRNP) are diagnostic for systemic lupus erythematosus (SLE) and play an important role in the pathogenesis of the disease. SLE affects more than a million people in the United States alone, primarily young and middle-aged women. SLE is a relapsing, remitting disease with devastating consequences and is poorly treated or prevented with existing therapies. Patients suffer from kidney dysfunction, leading to renal failure and a wide and variable range of symptoms including arthritis, fever, skin rashes, and brain inflammation. Increased serum levels of IFN-α have been observed in many SLE patients and correlate with both disease activity and key disease markers such as anti-DNA antibodies (Hooks et al., N. Engl. J. Med., 301:5-8 (1979); Ytterberg et al., Arthritis Rheum., 25:401-406 (1982); Bengtsson et al., Lupus, 9:664-671 (2000); Ronnblom et al., Arthritis Res. Ther., 5:68-75 (2003)).

Furthermore, a set of characteristic IFN-α-inducible genes are constitutively up-regulated in blood cells of SLE patients (Blanco et al., Science, 294:1540-1543 (2001); von Wussow et al., Arthritis Rheum., 32:914-918 (1989); Bennett et al., J. Exp. Med., 197:711-723 (2003); Baechler et al., Proc. Natl. Acad. Sci. USA, 100:2610-2615 (2003)). These elevated IFN-α levels have a direct role in the pathology of lupus because patients with non-autoimmune disorders who are treated with IFN-α develop antinuclear antibodies, anti-dsDNA antibodies, and SLE. Viral infections, UV skin injury, or other events leading to IFN-α induction are known to be activators of flares of SLE. In addition, NZB mice, which spontaneously develop a lupus-like disease, have less severe disease with delayed onset when made deficient for the IFN-α receptor (Santiago-Raber et al., J. Exp. Med., 197:777-788 (2003)).

There is considerable evidence that chronically activated plasmacytoid predendritic cells (PDCs) and the IFN-α that they produce in response to TLR stimulation are involved in the pathogenesis of SLE. For example, patients with SLE have a 50-100-fold decrease in the number of PDCs circulating in the blood (Blanco et al., Science, 294:1540-1543 (2001); Cederblad et al., J Autoimmun., 11:465-470 (1998)). This decrease is caused by in vivo activation of PDCs followed by cell migration into peripheral lymphoid tissues and sites of inflammation. Indeed, activated PDCs have been observed to accumulate in cutaneous lupus erythematosus lesions (Farkas et al., Am. J. Pathol., 159:237-243 (2001); Blomberg et al., Lupus, 10:484-490 (2001)). These cells, when activated with viruses, can produce large amounts of IFN-α. In addition, immune complexes of autoantibodies present in serum samples from SLE patients can cause the production of IFN-α by peripheral blood mononuclear cells (PBMCs) in vitro.

A growing body of evidence supports the idea that TLR activation plays a central role in the maintenance and progression of SLE by promoting elevated IFN-α levels. TLR7 and TLR9 are particularly relevant to SLE, as they are expressed by human PDCs, and stimulation through these receptors leads to very high levels of IFN-α production by PDCs. Exogenous viruses acting through these TLRs also induce IFN-α production and thus exacerbate the disease. Excessive IFN-α production in SLE can also be triggered by immune complexes of autoantibodies containing self-DNA or RNA (Ronnblom et al., Arthritis Res. Ther., 5:68-75 (2003); Vallin et al., J. Immunol., 163:6306-6313 (1999); Bave et al., J. Immunol., 165:3519-3526 (2000)). The recognition by TLRs is likely facilitated by the expression of FcγRII on PDCs, allowing efficient uptake of the self-nucleic acid into endosomal compartments that contain TLR7 and TLR 9 (Means et al., J. Clin. Invest., 115:407-417 (2005); Bave et al., J. Immunol., 171:3296-3302 (2003)).

Mammalian RNA or DNA, when complexed with autoantibodies, can represent potent self-antigens for TLR7 or TLR9, respectively. In fact, this inappropriate self-recognition by the innate immune system plays a substantial role in autoimmune diseases such as SLE. Several distinct subsets of atypical, non-stimulatory DNA sequences that inhibit TLR9 stimulation by CpG-containing immunostimulatory sequences have been described. These sequences have been identified from diverse sources, including viral sequences, mutated CpG sequences, and repeats of the TTAGGG motif present in mammalian telomeres (Krieg et al., Proc. Natl. Acad. Sci. USA, 95:12631-12636 (1998); Yamada et al., J. Immunol., 169:5590-5594 (2002); Zhu et al., J Leukoc. Biol., 72:1154-1163 (2002); Stunz et al., Eur. J. Immunol., 32:1212-1222 (2002); Ho et al., J. Immunol., 171:4920-4926 (2003); Gursel et al., J. Immunol., 171:1393-1400 (2003); Duramad et al., J. Immunol., 174:5193-5200 (2005); Zeuner et al., Arthritis Rheum., 46:2219-2224 (2002); Dong et al., Arthritis Rheum., 50:1686-1689 (2004)). Such TLR9 antagonists are active on human cells and can inhibit IFN-α production from PDCs in response to TLR9 activation.

Despite the identification of a variety of DNA sequences that can inhibit TLR9 signaling, RNA sequences that are capable of selectively inhibiting TLR7-mediated activation have not been identified. In addition, poor uptake of exogenous nucleic acids by cells represents a barrier to the development of DNA- or RNA-based inhibitors of TLR7 activation.

Thus, there is a strong need in the art for modulators of TLR7 signaling that reduce or completely abrogate an immune response triggered by immunostimulatory RNA or by inflammatory or autoimmune diseases such as SLE and methods for efficiently introducing them into cells. The present invention addresses these and other needs.

### SUMMARY OF THE INVENTION

The present invention provides methods of modulating the activation of certain Toll-like receptors such as TLR7 and/or TLR8 (“TLR7/8”) using chemically modified nucleic acid molecules. The present invention also provides methods of using such modified nucleic acid molecules to treat diseases or disorders associated with TLR7/8 activation such as SLE. The present invention further provides compositions comprising a combination of modified nucleic acid molecules and nucleic acid molecules that silence expression of one or more target sequences. Methods of using such compositions to reduce or abolish target gene expression without inducing cytokine production are also provided.

The present invention is based, in part, upon the surprising discovery that nucleic acid molecules having 2′-O-methyl (2′OMe) modifications at uridine, guanosine, and/or adenosine residues can reduce or completely abrogate (i.e., “antagonize”) the immune response induced by TLR7/8 agonists, including immunostimulatory RNA. In particular, Examples 1 and 3 illustrate that potent reduction of cytokine production in response to TLR7/8 agonists can be achieved by administering one or more of the modified nucleic acid molecules described herein. As a result, patients suffering from diseases or disorders in which inappropriate TLR7/8 activation induces excessive cytokine production can benefit from therapy with the modified nucleic acid molecules of the present invention. Furthermore, Examples 2 and 3 illustrate that potent gene silencing can be achieved with a significant reduction in cytokine production when an immunostimulatory RNA that silences expression of a target sequence is administered in combination with one or more non-complementary modified nucleic acid molecules of the present invention. Thus, patients can experience the benefits of RNAi therapy without suffering the immunostimulatory side-effects associated with such therapy.

In one aspect, the present invention provides a method for modulating TLR activation comprising administering to a mammalian subject an effective amount of a nucleic acid having at least one modified nucleotide. In some embodiments, the modified nucleic acid comprises a DNA sequence in the form of an oligonucleotide (e.g., single-stranded DNA), duplex DNA, plasmid DNA, PCR product, or derivatives or combinations of these groups. In other embodiments, the modified nucleic acid comprises an RNA sequence in the form of an oligonucleotide (e.g., single-stranded RNA), duplex RNA, or derivatives or combinations of these groups. The modified nucleic acid can alternatively comprise a single- or double-stranded sequence having a mixture of deoxyribonucleotides, ribonucleotides, and derivatives thereof (e.g., single-stranded DNA/RNA hybrid). In certain instances, the modified nucleic acid comprises at least one, two, three, four, five, six, seven, eight, nine, ten, or more modified nucleobases, sugars, and/or internucleoside linkages in the nucleic acid sequence. In some instances, all of the nucleotides in the nucleic acid sequence contain modified nucleobases, sugars, and/or internucleoside linkages. Without being bound to any particular theory, the modified nucleic acid molecules described herein modulate TLR7/8 activation by antagonizing the immune response (e.g., cytokine production) mediated by these receptors.

The modified nucleic acid typically contains at least one 2′OMe nucleotide such as a 2′OMe purine or pyrimidine nucleotide and includes 2′OMe-uridine nucleotides, 2′OMe-guanosine nucleotides, and/or 2′OMe-adenosine nucleotides (see, e.g., U.S. Patent Publication No. 20070135372). The modified nucleic acid generally does not contain only 2′OMe-cytidine modifications, but may contain at least one 2′OMe-cytidine nucleotide in addition to 2′OMe-uridine, 2′OMe-guanosine, and/or 2′OMe-adenosine nucleotides. In certain instances, at least two, three, four, five, six, seven, eight, nine, ten, or more uridines in the modified nucleic acid are 2′OMe-uridines. Preferably, every uridine in the modified nucleic acid is a 2′OMe-uridine (“Umod”). In certain other instances, at least two, three, four, five, six, seven, eight, nine, ten, or more guanosines in the modified nucleic acid are 2′OMe-guanosines. Preferably, every guanosine in the modified nucleic acid is a 2′OMe-guanosine (“Gmod”). Alternatively, at least two, three, four, five, six, seven, eight, nine, ten, or more adenosines in the modified nucleic acid are 2′OMe-adenosines. Preferably, every adenosine in the modified nucleic acid is a 2′OMe-adenosine (“Amod”). The modified nucleic acid can comprise a sequence of about 5 to about 1000 nucleotides in length, e.g., about 5-500, 5-250, 5-100, 5-60, 5-50, 5-40, 5-30, 10-60, 10-50, 10-40, 10-30, 15-60, 15-50, 15-40, or 15-30 nucleotides in length.

In a related aspect, the present invention provides a method for treating a disease or disorder associated with TLR activation comprising administering to a mammalian subject an effective amount of a nucleic acid having at least one modified nucleotide. As described above, the modified nucleic acid can comprise a single- or double-stranded DNA, a single- or double-stranded RNA, or a single- or double-stranded DNA/RNA hybrid sequence having one or more modified nucleobases, sugars, and/or internucleoside linkages. Without being bound to any particular theory, the modified nucleic acid molecules described herein are particularly useful for treating diseases or disorders associated with inappropriate TLR7/8 activation because they antagonize the TLR7/8-mediated immune response (e.g., cytokine production) that results from disease pathogenesis. In certain instances, the disease or disorder associated with TLR7/8 activation is an autoimmune disease such as, for example, systemic lupus erythematosus (SLE), multiple sclerosis, or arthritis.

The modified nucleic acid typically contains at least one 2′OMe nucleotide such as a 2′OMe purine or pyrimidine nucleotide and includes 2′OMe-uridine nucleotides, 2′OMe-guanosine nucleotides, and/or 2′OMe-adenosine nucleotides. The modified nucleic acid generally does not contain only 2′OMe-cytidine modifications, but may contain at least one 2′OMe-cytidine nucleotide in addition to 2′OMe-uridine, 2′OMe-guanosine, and/or 2′OMe-adenosine nucleotides. In certain instances, at least two, three, four, five, six, seven, eight, nine, ten, or more uridines in the modified nucleic acid are 2′OMe-uridines. In certain other instances, at least two, three, four, five, six, seven, eight, nine, ten, or more guanosines in the modified nucleic acid are 2′OMe-guanosines. Alternatively, at least two, three, four, five, six, seven, eight, nine, ten, or more adenosines in the modified nucleic acid are 2′OMe-adenosines. Preferably, the modified nucleic acid comprises a Umod, Gmod, and/or Amod sequence. The modified nucleic acid can comprise a sequence of about 5 to about 1000 nucleotides in length, e.g., about 5-500, 5-250, 5-100, 5-60, 5-50, 5-40, 5-30, 10-60, 10-50, 10-40, 10-30, 15-60, 15-50, 15-40, or 15-30 nucleotides in length.

With regard to autoimmune diseases such as SLE, TLR9 activation may also play a role in disease maintenance and progression by promoting elevated cytokine (e.g., IFN-α) levels. As such, in some embodiments, the method for treating a disease or disorder associated with TLR activation further comprises administering to the mammalian subject an effective amount of a TLR9 antagonist. Suitable TLR9 antagonists include, but are not limited to, viral sequences, mutated CpG sequences, and repeats of the TTAGGG motif present in mammalian telomeres. See, e.g., Krieg et al., Proc. Natl. Acad. Sci. USA, 95:12631-12636 (1998); Yamada et al., J. Immunol., 169:5590-5594 (2002); Zhu et al., J Leukoc. Biol., 72:1154-1163 (2002); Stunz et al., Eur. J. Immunol., 32:1212-1222 (2002); Ho et al., J. Immunol., 171:4920-4926 (2003); Gursel et al., J. Immunol., 171:1393-1400 (2003); Duramad et al., J. Immunol., 174:5193-5200 (2005); Zeuner et al., Arthritis Rheum., 46:2219-2224 (2002); Dong et al., Arthritis Rheum., 50:1686-1689 (2004). Additional TLR9 antagonists that are suitable for use in the methods of the present invention are described in, e.g., U.S. Patent Publication No. 20050239733.

In another aspect, the present invention provides a modified nucleic acid comprising a sequence of about 5 to about 1000 nucleotides in length (e.g., about 5-500, 5-250, 5-100, 5-60, 5-50, 5-40, 5-30, 10-60, 10-50, 10-40, 10-30, 15-60, 15-50, 15-40, or 15-30 nucleotides in length), wherein at least one uridine in the nucleic acid is a modified uridine such as a 2′OMe-uridine. As described above, the modified nucleic acid can comprise a single- or double-stranded DNA, a single- or double-stranded RNA, or a single- or double-stranded DNA/RNA hybrid sequence having one or more modified nucleobases, sugars, and/or internucleoside linkages. In one embodiment, at least two, three, four, five, six, seven, eight, nine, ten, or more uridines in the modified nucleic acid are 2′OMe-uridines. In another embodiment, every uridine in the modified nucleic acid is a 2′OMe-uridine.

In a related aspect, the present invention provides a modified nucleic acid comprising a sequence of about 5 to about 1000 nucleotides in length (e.g., about 5-500, 5-250, 5-100, 5-60, 5-50, 5-40, 5-30, 10-60, 10-50, 10-40, 10-30, 15-60, 15-50, 15-40, or 15-30 nucleotides in length), wherein at least one guanosine in the nucleic acid is a modified guanosine such as a 2′OMe-guanosine. As described above, the modified nucleic acid can comprise a single- or double-stranded DNA, a single- or double-stranded RNA, or a single- or double-stranded DNA/RNA hybrid sequence having one or more modified nucleobases, sugars, and/or internucleoside linkages. In one embodiment, at least two, three, four, five, six, seven, eight, nine, ten, or more guanosines in the modified nucleic acid are 2′OMe-guanosines. In another embodiment, every guanosine in the modified nucleic acid is a 2′OMe-guanosine.

In another related aspect, the present invention provides a modified nucleic acid comprising a sequence of about 5 to about 1000 nucleotides in length (e.g., about 5-500, 5-250, 5-100, 5-60, 5-50, 5-40, 5-30, 10-60, 10-50, 10-40, 10-30, 15-60, 15-50, 15-40, or 15-30 nucleotides in length), wherein at least one adenosine in the nucleic acid is a modified adenosine such as a 2′OMe-adenosine. As described above, the modified nucleic acid can comprise a single- or double-stranded DNA, a single- or double-stranded RNA, or a single- or double-stranded DNA/RNA hybrid sequence having one or more modified nucleobases, sugars, and/or internucleoside linkages. In one embodiment, at least two, three, four, five, six, seven, eight, nine, ten, or more adenosines in the modified nucleic acid are 2′OMe-adenosines. In another embodiment, every adenosine in the modified nucleic acid is a 2′OMe-adenosine.

In yet another aspect, the present invention provides a composition comprising a nucleic acid having at least one modified nucleotide and a nucleic acid that silences expression of a target sequence. As described above, the modified nucleic acid can comprise a single- or double-stranded DNA, a single- or double-stranded RNA, or a single- or double-stranded DNA/RNA hybrid sequence having one or more modified nucleobases, sugars, and/or internucleoside linkages. Without being bound to any particular theory, the modified nucleic acid molecules described herein modulate the immunostimulatory activity of nucleic acids that silence target gene expression by antagonizing the TLR7/8-mediated immune response (e.g., cytokine production) induced by such nucleic acids.

The modified nucleic acid typically contains at least one 2′OMe nucleotide such as a 2′OMe purine or pyrimidine nucleotide and includes 2′OMe-uridine nucleotides, 2′OMe-guanosine nucleotides, and/or 2′OMe-adenosine nucleotides. The modified nucleic acid generally does not contain only 2′OMe-cytidine modifications, but may contain at least one 2′OMe-cytidine nucleotide in addition to 2′OMe-uridine, 2′OMe-guanosine, and/or 2′OMe-adenosine nucleotides. In certain instances, at least two, three, four, five, six, seven, eight, nine, ten, or more uridines in the modified nucleic acid are 2′OMe-uridines. In certain other instances, at least two, three, four, five, six, seven, eight, nine, ten, or more guanosines in the modified nucleic acid are 2′OMe-guanosines. Alternatively, at least two, three, four, five, six, seven, eight, nine, ten, or more adenosines in the modified nucleic acid are 2′OMe-adenosines. Preferably, the modified nucleic acid comprises a Umod, Gmod, and/or Amod sequence. The modified nucleic acid can comprise a sequence of about 5 to about 1000 nucleotides in length, e.g., about 5-500, 5-250, 5-100, 5-60, 5-50, 5-40, 5-30, 10-60, 10-50, 10-40, 10-30, 15-60, 15-50, 15-40, or 15-30 nucleotides in length.

In some embodiments, the modified nucleic acid sequence does not have complementarity to the nucleic acid that silences expression of a target sequence. As a result, the modified nucleic acid typically does not hybridize to the nucleic acid that silences expression of a target sequence under stringent or moderately stringent hybridization conditions. Preferably, the nucleic acid that silences expression of a target sequence is an antisense oligonucleotide or small-interfering RNA (siRNA).

In other embodiments, the nucleic acid that silences expression of a target sequence comprises unmodified nucleotides. In certain instances, the unmodified nucleic acid sequence comprises at least one, two, three, four, five, six, seven, or more 5′-GU-3′ motifs. With regard to duplex nucleic acid (e.g., siRNA) sequences, the 5′-GU-3′ motif can be in the sense strand, the antisense strand, or both strands. In further embodiments, the nucleic acid that silences expression of a target sequence comprises at least one modified nucleotide. For example, one or more of the modified nucleobases, sugars, and/or internucleoside linkages described herein can be introduced into the sense and/or antisense strand of an siRNA sequence or into an antisense RNA oligonucleotide sequence.

In still other embodiments, the nucleic acid that silences expression of a target sequence has immunostimulatory activity. Immunostimulatory nucleic acid molecules usually comprise unmodified nucleotides and, in certain instances, at least one 5′-GU-3′ motif. Such molecules typically stimulate an immune response by inducing cytokine production (e.g., IFN-α, IFN-γ, TNF-α, IL-6, and/or IL-12).

The present invention also provides a pharmaceutical composition comprising a modified nucleic acid, a nucleic acid that silences expression of a target sequence, and a pharmaceutically acceptable carrier.

In still yet another aspect, the present invention provides a nucleic acid-lipid particle comprising a modified nucleic acid, a cationic lipid, and a non-cationic lipid. In certain instances, the nucleic acid-lipid particle further comprises a nucleic acid that silences expression of a target sequence. The nucleic acid-lipid particle can also comprise a conjugated lipid that inhibits aggregation of particles.

The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), or a mixture thereof. The cationic lipid may comprise from about 20 mol % to about 50 mol % or about 40 mol % of the total lipid present in the particle.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may comprise from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles may be from 0 mol % to about 20 mol % or about 2 mol % of the total lipid present in the particle.

In some embodiments, the nucleic acid-lipid particle further comprises cholesterol at, e.g., about 10 mol % to about 60 mol % or about 48 mol % of the total lipid present in the particle.

The modified nucleic acid can be fully encapsulated in the nucleic acid-lipid particle and/or complexed to the lipid portion of the particle. When the nucleic acid-lipid particle further comprises a nucleic acid that silences expression of a target sequence, both nucleic acid molecules (i.e., the modified nucleic acid and nucleic acid that silences target gene expression) are fully co-encapsulated in the nucleic acid-lipid particle and/or complexed to the lipid portion of the particle.

The present invention further provides a pharmaceutical composition comprising the nucleic acid-lipid particle and a pharmaceutically acceptable carrier.

In a further aspect, the modified nucleic acid and nucleic acid that silences target gene expression are used in methods for silencing expression of a target sequence. An effective amount of both nucleic acid molecules is administered to a mammalian subject, thereby silencing expression of a target sequence without inducing an immune response. Preferably, the mammalian subject is a human. In certain instances, both nucleic acid molecules are in a nucleic acid-lipid particle comprising a cationic lipid and a non-cationic lipid. In certain other instances, the nucleic acid-lipid particle can further comprise a conjugated lipid that inhibits aggregation of particles. Both nucleic acid molecules can be fully co-encapsulated in the nucleic acid-lipid particle and/or complexed to the lipid portion of the particle.

In an additional aspect, the present invention provides isolated nucleic acid molecules comprising a sequence set forth in Tables 1-3 or a modified version thereof (e.g., a nucleic acid molecule having one or more 2′OMe modifications).

Other features, objects, and advantages of the invention and its preferred embodiments will become apparent from the detailed description, examples, and claims that follow.

### BRIEF DESCRIPTION OF THE DRAWINGS

FIG. 1 shows that SNALP-encapsulated Luc UmodS ssRNA dose-dependently decreased the level of IFN-α induced by naked loxoribine (TLR7 agonist).

FIG. 2 shows that Luc UmodS ssRNA significantly reduced both IFN-α and IL-6 levels induced by naked RNA40 (TLR7/8 agonist) when both components were co-encapsulated in equimolar amounts in the same SNALP.

FIG. 3 shows that SNALP-encapsulated Luc UmodS ssRNA increased the level of IFN-α induced by naked ODN2216 (TLR9 agonist).

FIG. 4 shows that there was no reduction in IL-6 levels induced by naked ODN2006 (TLR9 agonist) when PBMCs were pretreated with SNALP-encapsulated Luc UmodS ssRNA.

FIG. 5 shows that SNALP-encapsulated Luc UmodS or polyUmod21 ssRNA inhibited both IFN-β and IL-6 levels induced by naked loxoribine.

FIG. 6 shows that the immunostimulatory effects of ApoB siRNA were significantly reduced when UmodS ssRNA was co-formulated with the siRNA in the same SNALP.

FIG. 7 shows that the presence of UmodS ssRNA in the same SNALP as an ApoB antisense ssRNA abolished the immunostimulatory activity of the antisense ssRNA.

FIG. 8 shows that the immunostimulatory effects of ApoB antisense ssRNA were antagonized when NP1496 UmodS ssRNA and ApoB antisense ssRNA were co-formulated in the same SNALP at a 1:1, 1:2, or 1:4 molar ratio.

FIG. 9 shows that high levels of IFN-α were induced when an unmodified sense strand ssRNA and an ApoB antisense ssRNA were co-formulated in the same SNALP.

FIG. 10 shows that high levels of IFN-α were induced when an unmodified β-gal sense strand ssRNA and an ApoB antisense ssRNA were co-formulated in the same SNALP.

FIG. 11 shows that polyUmod10, polyUmod15, and polyUmod21 ssRNA significantly reduced both IFN-α and IL-6 levels induced by β-gal antisense ssRNA when the modified ssRNA and antisense ssRNA were co-encapsulated in equimolar amounts in the same SNALP.

FIG. 12 shows that 2′OMe RNA inhibits RNA-mediated IFN-α and IL-6 production from human PBMCs. Interferon-α (IFN-α) responses from human PBMCs following treatment with (A,B,D-F) immunostimulatory ssRNA or (C) siRNA duplexes either alone or co-formulated with 2′OMe RNA. Robust IFN-α induction by (A) 1 μg/ml ApoB1 AS or (B) 0.15 μg/ml βgal AS ssRNA and (C) 1.5 μg/ml ApoB1 duplex RNA is abrogated by co-administration of the 2′OMe-uridine RNAs NP-mU or Luc-mU at equimolar concentrations. (D) The 2′OMe-cytidine RNAs NP-mC or βgal-mC 2′OMe-cytidine RNA do not inhibit IFN-α induction by 1 μg/ml ApoB1 AS ssRNA. (E) 2′OMe-guanosine (βgal-mG) and 2′OMe-adenosine (βgal-mA) modified RNA but not 2′OMe-cytidine (βgal-mC) modified RNA inhibits IFN-α induction by 0.5 μg/ml NP ssRNA. (F) 2′OMe-uridine homopolymers (mU)₂₁, (mU)₁₅, and (mU)₁₀ of 21, 15, and 10 nucleotides in length, respectively, inhibit IFN-α induction by 0.15 μg/ml βgal AS ssRNA. In each experiment, RNA molecules were administered either alone or at a 1:1 molar ratio of immunostimulatory:2′OMe RNA. Inhibition of cytokine induction was observed at all RNA doses tested from 0.1 to 3 μg/ml. Results for IL-6 in all experiments were equivalent to those for IFN-α. Data represent mean IFN-α in supernatants after 24 h culture+SD of triplicate wells and are representative of at least two separate experiments.

FIG. 13 shows that 2′OMe RNA inhibits RNA-mediated IFN-α and IL-6 production from murine Flt3L DCs. ssRNA (βgal AS) and 2′OMe-uridine 21mer homopolymer (mU)₂₁ were co-formulated in lipid nanoparticles at 1:1 molar ratios. Formulated RNA were added to murine Flt3L dendritic cells in 96 well triplicates at a concentration of 5 μg/ml βgal AS RNA. Supernatants were harvested 24 h later and analyzed for (A) IFN-α or (B) IL-6 by ELISA. Experiments were performed at least twice and two representative experiments are shown. Data represent mean pg/ml cytokine+SD of triplicate cultures.

FIG. 14 shows that 2′OMe RNA does not inhibit cytokine production by Type B or C ODN or polyIC in vitro. IFN-α responses from (A-C) murine Flt3L DCs and (D-E) human PBMCs treated with either lipid formulated CpG ODN alone or in combination with the 2′OMe RNAs (mU)₂₁ or Luc-mU. CpG ODN tested were (A) Type B ODN 1826, (B,D) Type C ODN M362, and (C,E) Type A ODN 6295 or 2216, respectively. Cells were treated with 0.5 μg/ml ODN alone or with an equimolar amount of the indicated 2′OMe RNA. 2′OMe RNA did not inhibit IFN-α induction by Type B or C ODN; however, responses to Type A ODN were significantly reduced. (F) IL-6 induction by human PBMCs treated with soluble polyIC (10 μg/ml) plus either native GFP-S ssRNA, Luc-mU, or (mU)₂₁ 2′OMe RNA (1.4 μg/ml) or lipid vehicle alone. In each experiment, data are mean pg/ml cytokines+SD of triplicate cultures 24 h after treatment and are representative of at least 2 independent experiments.

FIG. 15 shows that 2′OMe ssRNA inhibits loxoribine-mediated IFN-α and IL-6 production in both human and murine systems in vitro. Cytokine responses from (A,B) human PBMCs or (C,D) murine Flt3L DCs treated with the TLR7 agonist loxoribine at 300 μM or 30 μM, respectively. Cells were treated simultaneously with soluble loxoribine plus either media alone, lipid vehicle (lipid), or lipid formulated native ssRNA (GFP-S) or 2′OMe RNAs (Luc-mU or (mU)₂₁) for 24 h before (A,C) secreted IFN-α and (B,D) IL-6 were assayed. Control cultures received PBS vehicle only; RNA was added at 0.2 μM (˜1.4 μg/ml) final concentration. Data reflect mean cytokine levels+SD of triplicate cultures and are representative of at least two independent experiments.

FIG. 16 shows that 2′OMe RNA inhibits ssRNA and loxoribine-mediated cytokine production in vivo. (A) IFN-α and (B) IL-6 induction in mice treated with either immunostimulatory ssRNA (βgal AS), 2′OMe RNA ((mU)₂₁), or βgal AS+(mU)₂₁ co-formulated at a 1:1 molar ratio in lipid particles. Plasma cytokines were measured 6 h after IV administration of formulations containing 40 μg βgal AS RNA. (C-F) Treatment of mice with 2′OMe RNA inhibits (C,E) IFN-α and (D,F) IL-6 induction by loxoribine (Lox). Mice received 100 μg formulated (mU)₂₁, 2 h prior to the administration of 1 mg soluble Lox in PBS. Control groups were pre-treated with either (C,D) PBS or (E,F) 100 μg formulated GFP-S RNA, a native ssRNA with negligible immunostimulatory activity. Plasma IFN-α and IL-6 levels 2 h after IV Lox administration were significantly reduced in (mU)₂₁ treated mice compared to mice receiving either PBS or formulated control RNA. Data are mean +SD of n=4 mice per group and are representative of two separate experiments.

### DETAILED DESCRIPTION OF THE INVENTION

### I. Introduction

Toll-like receptors (TLRS) are a family of highly conserved polypeptides that play a critical role in innate immunity in mammals. Ten different TLRs have been identified in humans that recognize conserved microbial components, initiate specific biological responses, and are thus essential components of the innate response to infection. Interestingly, four of the ten TLRs have been implicated in the binding of nucleic acids. For example, TLR3 recognizes dsRNA from viruses and can also be stimulated by polyI:C, TLR7 and TLR8 recognize ssRNA, and TLR9 recognizes bacterial and viral DNA and synthetic oligonucleotides containing unmethylated CG dinucleotides (Janeway et al., Annu. Rev. Immunol., 20:197-216 (2002); Akira et al., Nat. Rev. Immunol., 4:499-511 (2004)).

The cytoplasmic domains of the various TLRs are characterized by a Toll-interleukin 1 (IL-1) receptor (TIR) domain (Medzhitov et al., Mol Cell, 2:253-258 (1998)). Recognition of microbial invasion by TLRs triggers activation of a signaling cascade that is evolutionarily conserved in Drosophila and mammals. The TIR domain-containing adapter protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines (Aderem et al, Nature, 406:782-787 (2000)).

TLRs are among the most widely expressed recognition receptors of the innate immune system. For example, human TLR7 is expressed in placenta, lung, spleen, lymph nodes, tonsil, and on plasmacytoid precursor dendritic cells (PDCs), while human TLR8 is expressed in lung, peripheral blood leukocytes (PBL), placenta, spleen, lymph nodes, and on monocytes (Chuang et al., Eur. Cytokine Net., 11:372-378 (2000); Kadowaki et al., J. Exp. Med., 194:863-869 (2001)). Human TLR9 is expressed in spleen, lymph nodes, bone marrow, PBL, and on PDCs and B cells (Chuang et al., supra; Kadowaki et al., supra; Bauer et al., Proc. Natl. Acad. Sci. USA, 98:9237-9242 (2001)).

TLRs are used by the innate immune system to discriminate between nucleic acids of mammalian versus microbial origin. However, the failure of TLRs to discriminate between self and nonself nucleic acids contributes to the development of inflammatory and autoimmune diseases. For example, patients with systemic lupus erythematosus (SLE) typically have immune complexes of autoantibodies to chromatin and RNA protein particles (snRNP). In fact, studies in a murine model of SLE in which the TLR7 gene is duplicated indicate that increased TLR7 expression may accelerate systemic autoimmunity by inducing activation of B cells by RNA-containing antigens of nucleolar origin (Pisitkun et al., Science, 312:1669-1672 (2006); Subramanian et al., Proc. Natl. Acad. Sci. USA, 103:9970-9975 (2006)). Furthermore, TLR7 is particularly relevant to SLE because stimulation through this receptor leads to very high levels of IFN-α production. As a result, mammalian RNA represents a potent self-antigen for TLR7 and induces the immune system to produce excessive amounts of cytokines.

It has recently been demonstrated that synthetic siRNA can be a potent activator of the innate immune response when administered with vehicles that facilitate intracellular delivery (Judge et al., Nat. Biotechnol., 23:457-462 (2005); Homung et al., Nat. Med., 11:263-270 (2005); Sioud, J. Mol. Biol., 348:1079-1090 (2005)). Immune recognition of siRNA is sequence-dependent and activates innate immune cells through the TLR7 pathway, causing potent induction of IFN-α and inflammatory cytokines. Toxicities associated with the administration of siRNA in vivo have been attributed to such a response (Morrissey et al., Nat. Biotechnol., 23:1002-1007 (2005); Judge et al., supra). This represents a significant barrier to the therapeutic development of RNAi due to toxicity and off-target gene effects associated with the inflammatory response.

The present invention provides, inter alia, nucleic acid molecules having 2′OMe modifications at one or more uridine, guanosine, and/or adenosine residues that can reduce or abrogate the immune response associated with inappropriate TLR7 and/or TLR8 (“TLR7/8”) activation by, for example, an immunostimulatory nucleic acid (e.g., unmodified siRNA or antisense oligonucleotide) or an inflammatory or autoimmune disease (e.g., SLE). Accordingly, treatment with a modified nucleic acid of the present invention has the potential to modulate TLR7, a major source of excessive IFN-α in autoimmune diseases such as SLE, without completely preventing the acute IFN-α responses to viral infection mediated by other recognition mechanisms such as TLR3 and protein kinase R. This approach could thus be less immunosuppressive than therapies aimed at blocking IFN-α interaction with its receptor. As such, the use of the modified nucleic acids described herein represents a new approach in the treatment of SLE to reduce symptoms and prevent relapses through inhibition of a key step in disease pathogenesis.

### II. Definitions

As used herein, the following terms have the meanings ascribed to them unless specified otherwise.

The term “nucleic acid” or “polynucleotide” refers to a polymer containing at least two deoxyribonucleotides or ribonucleotides having naturally-occurring or modified nucleobases or sugars in either single- or double-stranded form and includes DNA, RNA, hybrids thereof, and mimetics thereof. DNA may be in the form of, e.g., oligonucleotides (e.g., single-stranded DNA), plasmid DNA, pre-condensed DNA, a PCR product, vectors (P1, PAC, BAC, YAC, artificial chromosomes), expression cassettes, chimeric sequences, chromosomal DNA, or derivatives or combinations of these groups. RNA may be in the form of, e.g., oligonucleotides (e.g., single-stranded RNA), siRNA, mRNA, tRNA, rRNA, tRNA, vRNA, and combinations thereof. Nucleic acids include nucleic acids containing known nucleotide analogs or modified backbone residues or linkages, which are synthetic, naturally-occurring, and non-naturally-occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “interfering RNA” or “RNAi” or “interfering RNA sequence” refers to double-stranded RNA (i.e., duplex RNA) that is capable of reducing or inhibiting expression of a target gene (i.e., by mediating the degradation of mRNAs which are complementary to the sequence of the interfering RNA) when the interfering RNA is in the same cell as the target gene. Interfering RNA thus refers to the double-stranded RNA formed by two complementary strands or by a single, self-complementary strand. Interfering RNA may have substantial or complete identity to the target gene or may comprise a region of mismatch (i.e., a mismatch motif). The sequence of the interfering RNA can correspond to the full length target gene, or a subsequence thereof.

As used herein, the term “small-interfering RNA” or “siRNA,” refers to a double-stranded interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is about 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate oligonucleotides, wherein one strand is the sense strand and the other is the complementary antisense strand; a double-stranded polynucleotide molecule assembled from a single oligonucleotide, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight and Bass, Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The term “oligonucleotide” refers to a single-stranded oligomer or polymer of RNA, DNA, and/or a mimetic thereof. In certain instances, oligonucleotides are composed of naturally-occurring (i.e., unmodified) nucleobases, sugars, and internucleoside (backbone) linkages. In certain other instances, oligonucleotides comprise modified nucleobases, sugars, and/or internucleoside linkages.

An “antisense oligonucleotide” refers to a single-stranded oligomer or polymer of RNA, DNA, and/or a mimetic thereof which hybridizes to a complementary mRNA sequence. The antisense oligonucleotide typically comprises a nucleic acid sequence that is complementary to a subsequence of the mRNA. For example, the antisense oligonucleotide may correspond to the antisense strand of an siRNA duplex. In some embodiments, the antisense oligonucleotide interferes with the normal function of the mRNA by reducing or inhibiting its expression. Antisense oligonucleotides include, but are not limited to, antisense RNA, antisense DNA, ribozymes, external guide sequence (EGS) oligonucleotides (i.e., oligozymes), and short catalytic RNAs which hybridize to a target nucleic acid sequence and modulate its expression. Antisense oligonucleotides are preferably chemically synthesized.

As used herein, the term “mismatch motif” or “mismatch region” refers to a portion of a nucleic acid sequence that does not have 100% complementarity to its target sequence. A nucleic acid may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

An “effective amount” refers to an amount of a nucleic acid that is sufficient to bring about a desired biologic effect. For example, an effective amount of a modified nucleic acid is an amount that is sufficient to reduce or abrogate a TLR7/8-mediated immune response, while an effective amount of a nucleic acid that silences expression of a target sequence is an amount that is sufficient to reduce or abrogate target gene expression. An effective amount can but need not be limited to an amount administered in a single administration.

By “inhibiting,” “reducing,” or “antagonizing” an immune response is intended to mean a detectable decrease of an immune response in the presence of a modified nucleic acid. For example, the amount of decrease of an immune response may be determined relative to the level of immune stimulation in the absence of the modified nucleic acid. A detectable decrease can be about 5%, 10%, 15%, 20%, 25%, 30%, 35%, 40%, 45%, 50%, 55%, 60%, 65%, 70%, 75%, 80%, 85%, 90%, 95%, 100%, or more lower than the immune response detected in the absence of the modified nucleic acid. A decrease in the immune response is typically measured by a decrease in cytokine production (e.g., IFNα, IFNγ, TNFα, IL-6, and/or IL-12) by a responder cell in vitro or a decrease in cytokine production in the sera of a mammalian subject after administration of the modified nucleic acid.

As used herein, the term “responder cell” refers to a cell, preferably a mammalian cell, that produces a detectable immune response when contacted with an immunostimulatory nucleic acid or TLR agonist. Exemplary responder cells include, e.g., dendritic cells, macrophages, peripheral blood mononuclear cells (PBMC), splenocytes, and the like. Detectable immune responses include, e.g., production of cytokines or growth factors such as IFN-α, IFN-γ, TNF-α, TNF-β, IL-1, IL-2, IL-3, IL-4, IL-5, IL-6, IL-10, IL-12, IL-13, TGF, and combinations thereof.

“Substantial identity” refers to a sequence that hybridizes to a reference sequence under stringent conditions, or to a sequence that has a specified percent identity over a specified region of a reference sequence.

The phrase “stringent hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization.

Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, Ausubel et al., eds.

The terms “substantially identical” or “substantial identity,” in the context of two or more nucleic acids, refer to two or more sequences or subsequences that are the same or have a specified percentage of nucleotides that are the same (i.e., at least about 60%, preferably at least about 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 5, 10, 15, 20, 25, 30, 35, 40, 45, 50, 75, or 100 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of a number of contiguous positions selected from the group consisting of from about 20 to about 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math., 2:482 (1981), by the homology alignment algorithm of Needleman and Wunsch, J. Mol. Biol., 48:443 (1970), by the search for similarity method of Pearson and Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. (1995 supplement)).

Examples of algorithms that are suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. Software for performing BLAST analyses is publicly available through the National Center for Biotechnology Information (www.ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, Henikoff and Henikoff, Proc. Natl. Acad. Sci. USA, 89:10915 (1989)).

The BLAST algorithm also performs a statistical analysis of the similarity between two sequences (see, e.g., Karlin and Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

“Lipid vesicle” refers to any lipid composition that can be used to deliver a compound such as a nucleic acid including, but not limited to, liposomes, wherein an aqueous volume is encapsulated by an amphipathic lipid bilayer; or wherein the lipids coat an interior comprising a large molecular component, such as a plasmid, with a reduced aqueous interior; or lipid aggregates or micelles, wherein the encapsulated component is contained within a relatively disordered lipid mixture. The term lipid vesicle encompasses any of a variety of lipid-based carrier systems including, without limitation, SPLPs, pSPLPs, SNALPs, liposomes, micelles, virosomes, lipid-nucleic acid particles, nucleic acid complexes, and mixtures thereof.

As used herein, “lipid encapsulated” can refer to a lipid formulation that provides a compound such as a nucleic acid with full encapsulation or partial encapsulation. In a preferred embodiment, one or more nucleic acids are fully encapsulated in the lipid formulation (e.g., to form a SPLP, pSPLP, SNALP, or other nucleic acid-lipid particle).

As used herein, the term “SNALP” refers to a stable nucleic acid-lipid particle, including SPLP. A SNALP represents a vesicle of lipids coating a reduced aqueous interior comprising a nucleic acid (e.g., ssRNA, antisense oligonucleotide, siRNA, ssDNA, dsDNA, micro RNA (miRNA), short hairpin RNA (shRNA), dsRNA, and/or a plasmid). As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which comprise an encapsulated condensing agent-nucleic acid complex as set forth in PCT Publication No. WO 00/03683.

The nucleic acid-lipid particles of the present invention typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

The term “vesicle-forming lipid” is intended to include any amphipathic lipid having a hydrophobic moiety and a polar head group, and which by itself can form spontaneously into bilayer vesicles in water, as exemplified by most phospholipids.

The term “vesicle-adopting lipid” is intended to include any amphipathic lipid that is stably incorporated into lipid bilayers in combination with other amphipathic lipids, with its hydrophobic moiety in contact with the interior, hydrophobic region of the bilayer membrane, and its polar head group moiety oriented toward the exterior, polar surface of the membrane. Vesicle-adopting lipids include lipids that on their own tend to adopt a nonlamellar phase, yet which are capable of assuming a bilayer structure in the presence of a bilayer-stabilizing component. A typical example is dioleoylphosphatidylethanolamine (DOPE). Bilayer stabilizing components include, but are not limited to, conjugated lipids that inhibit aggregation of nucleic acid-lipid particles, polyamide oligomers (e.g., ATTA-lipid derivatives), peptides, proteins, detergents, lipid-derivatives, PEG-lipid derivatives such as PEG coupled to dialkyloxypropyls, PEG coupled to diacylglycerols, PEG coupled to phosphatidyl-ethanolamines, PEG conjugated to ceramides (see, e.g., U.S. Pat. No. 5,885,613), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties.

The term “amphipathic lipid” refers, in part, to any suitable material wherein the hydrophobic portion of the lipid material orients into a hydrophobic phase, while the hydrophilic portion orients toward the aqueous phase. Amphipathic lipids are usually the major component of a lipid vesicle. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipid described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any neutral lipid as described above as well as anionic lipids.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “cationic lipid” refers to any of a number of lipid species that carry a net positive charge at a selected pH, such as physiological pH. It has been surprisingly found that cationic lipids comprising alkyl chains with multiple sites of unsaturation, e.g., at least two or three sites of unsaturation, are particularly useful for forming nucleic acid-lipid particles with increased membrane fluidity. A number of cationic lipids and related analogs, which are also useful in the present invention, are described in U.S. Patent Publication No. 20060083780; U.S. Pat. Nos. 5,208,036; 5,264,618; 5,279,833; 5,283,185; 5,753,613; and 5,785,992; and PCT Publication No. WO 96/10390. Examples of cationic lipids include, but are not limited to, N,N-dioleyl-N,N-dimethylammonium chloride (DODAC), dioctadecyldimethylammonium (DODMA), distearyldimethylammonium (DSDMA), N-(2,3-dioleyloxy)propyl)-N,N,N-trimethylammonium chloride (DOTMA), N,N-distearyl-N,N-dimethylammonium bromide (DDAB), N-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 1,2-DiLinoleyloxy-N,N-dimethylaminopropane (DLinDMA), 1,2-Dilinolenyloxy-N,N-dimethylaminopropane (DLenDMA), and mixtures thereof. In some cases, the cationic lipids comprise a protonatable tertiary amine head group, C18 alkyl chains, ether linkages between the head group and alkyl chains, and 0 to 3 double bonds. Such lipids include, e.g., DSDMA, DLinDMA, DLenDMA, and DODMA. The cationic lipids may also comprise ether linkages and pH titratable head groups. Such lipids include, e.g., DODMA.

The term “hydrophobic lipid” refers to compounds having apolar groups that include, but are not limited to, long chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

The term “fusogenic” refers to the ability of a liposome, a SNALP, or other drug delivery system to fuse with membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism.

“Serum-stable” in relation to nucleic acid-lipid particles means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery that leads to a broad biodistribution of a compound such as a nucleic acid within an organism. Some techniques of administration can lead to the systemic delivery of certain compounds, but not others. Systemic delivery means that a useful, preferably therapeutic, amount of a compound is exposed to most parts of the body. Obtaining a broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery of nucleic acid-lipid particles can be by any means known in the art including, for example, intravenous, subcutaneous, and intraperitoneal. In a preferred embodiment, systemic delivery of nucleic acid-lipid particles is by intravenous delivery.

“Local delivery,” as used herein, refers to delivery of a compound such as a nucleic acid directly to a target site within an organism. For example, a nucleic acid can be locally delivered by direct injection into a disease site such as a tumor or other target site such as a site of inflammation or a target organ such as the liver, heart, pancreas, kidney, and the like.

The term “mammal” refers to any mammalian species such as a human, mouse, rat, dog, cat, hamster, guinea pig, rabbit, livestock, and the like.

The term “autoimmune disease” refers to a disease or disorder resulting from an immune response against a self tissue or tissue component and includes a self antibody response or cell-mediated response. The term encompasses non-organ specific autoimmune diseases, in which an autoimmune response is directed against a component present in several or many organs throughout the body. Such autoimmune diseases include, for example, systemic lupus erythematosus (SLE), progressive systemic sclerosis and variants, polymyositis, and dermatomyositis. The term also encompasses organ-specific autoimmune diseases, in which an autoimmune response is directed against a single tissue, such as Type I diabetes mellitus, myasthenia gravis, vitiligo, Graves' disease, Hashimoto's disease, Addison's disease, autoimmune gastritis, and autoimmune hepatitis. Additional autoimmune diseases include, but are not limited to, multiple sclerosis, pernicious anemia, primary biliary cirrhosis, autoimmune thrombocytopenia, and Sjögren's syndrome. Autoimmune diseases also include inflammatory diseases such as rheumatoid arthritis and other arthritic diseases.

The term “inflammatory disease” refers to a disease or disorder characterized or caused by inflammation. “Inflammation” refers to a local response to cellular injury that is marked by capillary dilatation, leukocytic infiltration, redness, heat, and pain that serves as a mechanism initiating the elimination of noxious agents and of damaged tissue. The site of inflammation includes the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, a follicle, and the like. Such inflammatory diseases include, but are not limited to, rheumatoid diseases (e.g., rheumatoid arthritis), other arthritic diseases (e.g., acute arthritis, acute gouty arthritis, bacterial arthritis, chronic inflammatory arthritis, degenerative arthritis (osteoarthritis), infectious arthritis, juvenile arthritis, mycotic arthritis, neuropathic arthritis, polyarthritis, proliferative arthritis, psoriatic arthritis, venereal arthritis, viral arthritis), inflammatory bowel disease (e.g., Crohn's disease, ulcerative colitis), fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, and blastomycosis. Rheumatoid arthritis is a chronic inflammatory disease primarily of the joints, usually polyarticular, marked by inflammatory changes in the synovial membranes and articular structures and by muscle atrophy and rarefaction of the bones.

As used herein, the term “agonist” refers to an agent that binds to a polypeptide or protein and stimulates, increases, activates, facilitates, enhances activation, sensitizes, or up-regulates the activity of the polypeptide or protein. In certain instances, the agonist binds to a Toll-like receptor (TLR) and affects its activity, e.g., by inducing cytokine production.

An “antagonist” refers to an agent that inhibits the activity of a polypeptide or protein or binds to, partially or totally blocks stimulation, decreases, prevents, delays activation, inactivates, desensitizes, or down-regulates the activity of the polypeptide or protein. In certain instances, the antagonist binds to a Toll-like receptor (TLR) and affects its activity, e.g., by reducing or abrogating cytokine production.

“Inhibitors,” “activators,” and “modulators” of activity are used herein to refer to inhibitory, activating, and modulating molecules, respectively, such as agonists, antagonists, ligands, mimetics, and their homologs and derivatives. The term “modulator” includes both inhibitors and activators. Inhibitors are agents that bind to, partially or totally block stimulation or activity, decrease, prevent, delay activation, inactivate, desensitize, or down-regulate the activity of a polypeptide or protein to a level that is about 5%, 10%, 15%, 20%, 25%, 30%, 35%, 40%, 45%, 50%, 55%, 60%, 65%, 70%, 75%, 80%, 85%, 90%, 95%, 100%, or more lower than in the absence of the inhibitor, e.g., TLR7/8 antagonist. Activators are agents that bind to, stimulate, increase, open, activate, facilitate, enhance activation or activity, sensitize, or up-regulate the activity of a polypeptide or protein to a level that is about 5%, 10%, 15%, 20%, 25%, 30%, 35%, 40%, 45%, 50%, 55%, 60%, 65%, 70%, 75%, 80%, 85%, 90%, 95%, 100%, or more higher than in the absence of the activator, e.g., TLR7/8 agonist. Modulators include naturally-occurring and synthetic antagonists, agonists, ligands, mimetics, small chemical molecules, antibodies, and the like.

The term “modulating Toll-like receptor activation” as used herein refers to activating (e.g., stimulating, increasing, facilitating, enhancing activation, sensitizing, up-regulating) or inhibiting (e.g., decreasing, preventing, partially or totally blocking, delaying activation, inactivating, desensitizing, down-regulating) Toll-like receptor signaling.

### III. Nucleic Acids

A. Modified Nucleic Acids

The modified nucleic acid molecules of the present invention can advantageously reduce or abrogate the immune response associated with inappropriate TLR7 and/or TLR8 (“TLR7/8”) activation by, e.g., an inflammatory disease, an autoimmune disease, or an immunostimulatory nucleic acid. The modified nucleic acid typically comprises a sequence of about 5 to about 1000 nucleotides in length, e.g., about 5-500, 5-250, 5-100, 5-60, 5-50, 5-40, 5-30, 10-60, 10-50, 10-40, 10-30, 15-60, 15-50, 15-40, or 15-30 nucleotides in length. In one embodiment, at least two, three, four, five, six, seven, eight, nine, ten, or more uridines, guanosines, and/or adenosines in the nucleic acid are modified. In another embodiment, every uridine, guanosine, and/or adenosine in the nucleic acid is modified.

Examples of modified nucleotides suitable for use in the present invention and methods for chemically modifying nucleic acids are described in, e.g., U.S. Patent Publication No. 20070135372, and include ribonucleotides having a 2′-O-methyl (2′OMe), 2′-deoxy-2′-fluoro (2° F.), 2′-deoxy, 5-C-methyl, 2′-O-(2-methoxyethyl) (MOE), 4′-thio, 2′-amino, or 2′-C-allyl group. Nucleotides having a Northern conformation such as those described in, e.g., Saenger, Principles of Nucleic Acid Structure, Springer-Verlag Ed. (1984), are also suitable for use in the modified nucleic acid molecules of the present invention. Such modified nucleotides include, without limitation, locked nucleic acid (LNA) nucleotides (e.g., 2′-O, 4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2° F.) nucleotides, 2′-deoxy-2′-chloro (2′Cl) nucleotides, and 2′-azido nucleotides. In addition, nucleotides having a nucleotide base analog such as, for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (see, e.g., Loakes, Nucl. Acids Res., 29:2437-2447 (2001)) can be incorporated into the modified nucleic acid molecules of the present invention.

In certain instances, the modified nucleic acid comprises non-naturally occurring nucleotides as a percentage of the total number of nucleotides present in the nucleic acid molecule. For example, about 5%, 10%, 15%, 20%, 25%, 30%, 35%, 40%, 45%, 50%, 55%, 60%, 65%, 70%, 75%, 80%, 85%, 90%, 95%, or 100% of the nucleotides in the nucleic acid can comprise modified nucleotides.

In some embodiments, the modified nucleic acid molecules of the present invention further comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., Tetrahedron, 49:1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al., Nucleic Acid Analogues: Synthesis and Properties, in Modern Synthetic Methods, VCH, 331-417 (1995); Mesmaeker et al., Novel Backbone Replacements for Oligonucleotides, in Carbohydrate Modifications in Antisense Research, ACS, 24-39 (1994)).

Additional examples of modified nucleotides and types of chemical modifications that can be introduced into the modified nucleic acid molecules of the present invention are described, e.g., in UK Patent No. GB 2,397,818 B and U.S. Patent Publication Nos. 20040192626, 20050282188, and 20050239733.

The modified nucleic acid molecules of the present invention can optionally comprise one or more non-nucleotides. As used herein, the term “non-nucleotide” refers to any group or compound that can be incorporated into a nucleic acid chain in the place of one or more nucleotide units, including sugar and/or phosphate substitutions, and allows the remaining bases to exhibit their activity. The group or compound is abasic in that it does not contain a commonly recognized nucleotide base such as adenosine, guanine, cytosine, uracil, or thymine and therefore lacks a base at the 1′-position.

In other embodiments, chemical modification of the nucleic acid comprises attaching a conjugate to the chemically-modified nucleic acid molecule. The conjugate can be attached at the 5′ and/or 3′-end of the chemically-modified nucleic acid via a covalent attachment such as, e.g., a biodegradable linker. The conjugate can also be attached to the chemically-modified nucleic acid, e.g., through a carbamate group or other linking group (see, e.g., U.S. Patent Publication Nos. 20050074771, 20050043219, and 20050158727). In certain instances, the conjugate is a molecule that facilitates the delivery of the chemically-modified nucleic acid into a cell. Examples of conjugate molecules suitable for attachment to the chemically-modified nucleic acid molecules of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Yet other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the chemically-modified nucleic acid molecule can be evaluated for improved pharmacokinetic profiles, bioavailability, and/or stability of the modified nucleic acid while retaining TLR7/8 modulating activity. As such, one skilled in the art can screen chemically-modified nucleic acid molecules having various conjugates attached thereto to identify ones having improved properties and substantial TLR7/8 modulating activity using any of a variety of well-known in vitro cell culture or in vivo animal models.

Preferably, the modified nucleic acid molecules of the present invention are chemically synthesized. For example, the oligonucleotides that comprise the modified nucleic acid molecules of the present invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol with a 2.5 min. coupling step for 2′-O-methylated nucleotides. Alternatively, syntheses at the 0.2 μmol scale can be performed on a 96-well plate synthesizer from Protogene (Palo Alto, Calif.). However, a larger or smaller scale of synthesis is also within the scope of the present invention. Suitable reagents for oligonucleotide synthesis, methods for nucleic acid deprotection, and methods for nucleic acid purification are known to those of skill in the art.

In addition to its utility in antagonizing an immune response induced by TLR7/8 activation, the modified nucleic acid molecules described herein are also useful in research and development applications as well as diagnostic, prophylactic, prognostic, clinical, and other healthcare applications.

B. siRNAs

The siRNA molecules of the present invention are capable of silencing expression of a target sequence, are about 15 to 60 or about 15 to 30 nucleotides in length, and are typically immunostimulatory. The siRNA sequences may have 3′ overhangs of one, two, three, four, or more nucleotides on one or both sides of the double-stranded region, or may lack overhangs (i.e., have blunt ends). Preferably, the siRNA sequences have 3′ overhangs of two nucleotides on each side of the double-stranded region. In certain instances, the 3′ overhang on the antisense strand has complementarity to the target sequence and the 3′ overhang on the sense strand has complementarity to the complementary strand of the target sequence. Alternatively, the 3′ overhangs do not have complementarity to the target sequence or the complementary strand thereof. Examples of such 3′ overhangs include, but are not limited to, 3′ deoxythymidine (dT) overhangs of one, two, three, four, or more nucleotides.

According to the methods of the present invention, siRNA molecules which are immunostimulatory can be introduced into cells in combination with modified nucleic acid molecules (e.g., Umod, Gmod, and/or Amod nucleic acid sequences) to reduce or completely abrogate their immunostimulatory properties without having a negative impact on RNAi activity. An immunostimulatory siRNA typically comprises naturally-occurring (i.e., unmodified) nucleobases, sugars, and internucleoside (backbone) linkages, but can also include one or more modified nucleobases, sugars, and/or internucleoside linkages in the sense and/or antisense strand.

1. Selection of siRNA Sequences

Suitable siRNA sequences can be identified using any means known in the art. Typically, the methods described in Elbashir et al., Nature 411:494-498 (2001) and Elbashir et al., EMBO J, 20:6877-6888 (2001) are combined with rational design rules set forth in Reynolds et al., Nature Biotech., 22:326-330 (2004).

Generally, the nucleotide sequence 3′ of the AUG start codon of a transcript from the target gene of interest is scanned for dinucleotide sequences (e.g., AA, NA, CC, GG, or UU, wherein N═C, G, or U) (see, e.g., Elbashir et al., EMBO J, 20:6877-6888 (2001)). The nucleotides immediately 3′ to the dinucleotide sequences are identified as potential siRNA sequences (i.e., a target sequence or a sense strand sequence). Typically, the 19, 21, 23, 25, 27, 29, 31, 33, 35, or more nucleotides immediately 3′ to the dinucleotide sequences are identified as potential siRNA sequences. In some embodiments, the dinucleotide sequence is an AA or NA sequence and the 19 nucleotides immediately 3′ to the AA or NA dinucleotide are identified as a potential siRNA sequences. siRNA sequences are usually spaced at different positions along the length of the target gene. To further enhance silencing efficiency of the siRNA sequences, potential siRNA sequences may be analyzed to identify sites that do not contain regions of homology to other coding sequences, e.g., in the target cell or organism. For example, a suitable siRNA sequence of about 21 base pairs typically will not have more than 16-17 contiguous base pairs of homology to coding sequences in the target cell or organism. If the siRNA sequences are to be expressed from an RNA Pol III promoter, siRNA sequences lacking more than 4 contiguous A's or T's are selected.

Once a potential siRNA sequence has been identified, a complementary sequence (i.e., an antisense strand sequence) can be designed. A potential siRNA sequence can also be analyzed using a variety of criteria known in the art. For example, to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. siRNA design tools that incorporate algorithms that assign suitable values of each of these features and are useful for selection of siRNA can be found at, e.g., http://boz094.ust.hk/RNAi/siRNA. One of skill in the art will appreciate that sequences with one or more of the foregoing characteristics may be selected for further analysis and testing as potential siRNA sequences.

Additionally, potential siRNA sequences with one or more of the following criteria can often be eliminated as siRNA: (1) sequences comprising a stretch of 4 or more of the same base in a row; (2) sequences comprising homopolymers of Gs (i.e., to reduce possible non-specific effects due to structural characteristics of these polymers; (3) sequences comprising triple base motifs (e.g., GGG, CCC, AAA, or TTT); (4) sequences comprising stretches of 7 or more G/Cs in a row; and (5) sequences comprising direct repeats of 4 or more bases within the candidates resulting in internal fold-back structures. However, one of skill in the art will appreciate that sequences with one or more of the foregoing characteristics may still be selected for further analysis and testing as potential siRNA sequences.

In some embodiments, potential siRNA sequences may be further analyzed based on siRNA duplex asymmetry as described in, e.g., Khvorova et al., Cell, 115:209-216 (2003); and Schwarz et al., Cell, 115:199-208 (2003). In other embodiments, potential siRNA sequences may be further analyzed based on secondary structure at the mRNA target site as described in, e.g., Luo et al., Biophys. Res. Commun., 318:303-310 (2004). For example, mRNA secondary structure can be modeled using the Mfold algorithm (available at http://www.bioinfo.rpi.edu/applications/mifold/ma/forml.cgi) to select siRNA sequences which favor accessibility at the mRNA target site where less secondary structure in the form of base-pairing and stem-loops is present.

Once a potential siRNA sequence has been identified, the sequence can be analyzed for the presence of any immunostimulatory properties, e.g., using an in vitro cytokine assay or an in vivo animal model. Motifs in the sense and/or antisense strand of the siRNA sequence such as GU-rich motifs (e.g., 5′-GU-3′,5′-UGU-3′,5′-GUGU-3′,5′-UGUGU-3′, etc.) can also provide an indication of whether the sequence may be immunostimulatory. As a non-limiting example, the siRNA sequence can be contacted with a mammalian responder cell under conditions such that the cell produces a detectable immune response to determine whether the siRNA is an immunostimulatory or a non-immunostimulatory siRNA. The mammalian responder cell may be from a naïve mammal (i.e., a mammal that has not previously been in contact with the gene product of the siRNA sequence). The mammalian responder cell may be, e.g., a peripheral blood mononuclear cell (PBMC), a macrophage, and the like. The detectable immune response may comprise production of a cytokine or growth factor such as, e.g., TNF-α, TNF-β, IFN-α, IFN-β, IFN-γ, IL-6, IL-12, or a combination thereof. An siRNA molecule identified as being immunostimulatory can then be introduced into a mammalian responder cell in combination with a modified nucleic acid to determine whether the modified nucleic acid can reduce or abrogate its immunostimulatory properties.

Suitable in vitro assays for detecting an immune response include, but are not limited to, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “Western blot” method of Gordon et al. (U.S. Pat. No. 4,452,901); immunoprecipitation of labeled ligand (Brown et al., J. Biol. Chem., 255:4980-4983 (1980)); enzyme-linked immunosorbent assays (ELISA) as described, for example, by Raines et al., J. Biol. Chem., 257:5154-5160 (1982); immunocytochemical techniques, including the use of fluorochromes (Brooks et al., Clin. Exp. Immunol., 39:477 (1980)); and neutralization of activity (Bowen-Pope et al., Proc. Natl. Acad. Sci. USA, 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

A non-limiting example of an in vivo model for detecting an immune response includes an in vivo mouse cytokine induction assay as described in, e.g., Judge et al., Mol. Ther., 13:494-505 (2006). In certain embodiments, the assay that can be performed as follows: (1) siRNA can be administered by standard intravenous injection in the lateral tail vein; (2) blood can be collected by cardiac puncture about 6 hours after administration and processed as plasma for cytokine analysis; and (3) cytokines can be quantified using sandwich ELISA kits according to the manufacturer's instructions (e.g., mouse and human IFN-α (PBL Biomedical; Piscataway, N.J.); human IL-6 and TNF-α (eBioscience; San Diego, Calif.); and mouse IL-6, TNF-α, and IFN-γ (BD Biosciences; San Diego, Calif.)).

Monoclonal antibodies that specifically bind cytokines and growth factors are commercially available from multiple sources and can be generated using methods known in the art (see, e.g., Kohler et al, Nature, 256: 495-497 (1975) and Harlow and Lane, ANTIBODIES, A LABORATORY MANUAL, Cold Spring Harbor Publication, New York (1999)). Generation of monoclonal antibodies has been previously described and can be accomplished by any means known in the art (Buhring et al., in Hybridoma, Vol. 10, No. 1, pp. 77-78 (1991)). In some methods, the monoclonal antibody is labeled (e.g., with any composition detectable by spectroscopic, photochemical, biochemical, electrical, optical, or chemical means) to facilitate detection.

2. Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. The siRNA sequences may have overhangs (e.g., 3′ or 5′ overhangs as described in Elbashir et al., Genes Dev., 15:188 (2001) or Nykänen et al, Cell, 107:309 (2001), or may lack overhangs (i.e., to have blunt ends).

An RNA population can be used to provide long precursor RNAs, or long precursor RNAs that have substantial or complete identity to a selected target sequence can be used to make the siRNA. The RNAs can be isolated from cells or tissue, synthesized, and/or cloned according to methods well known to those of skill in the art. The RNA can be a mixed population (obtained from cells or tissue, transcribed from cDNA, subtracted, selected, etc.), or can represent a single target sequence. RNA can be naturally occurring (e.g., isolated from tissue or cell samples), synthesized in vitro (e.g., using T7 or SP6 polymerase and PCR products or a cloned cDNA), or chemically synthesized.

To form a long dsRNA, for synthetic RNAs, the complement is also transcribed in vitro and hybridized to form a dsRNA. If a naturally occurring RNA population is used, the RNA complements are also provided (e.g., to form dsRNA for digestion by E. coli RNAse III or Dicer), e.g., by transcribing cDNAs corresponding to the RNA population, or by using RNA polymerases. The precursor RNAs are then hybridized to form double stranded RNAs for digestion. The dsRNAs can be directly administered to a subject or can be digested in vitro prior to administration.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols. A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

Preferably, siRNA are chemically synthesized. The oligonucleotides that comprise the siRNA molecules of the present invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope of the present invention. Suitable reagents for oligonucleotide synthesis, methods for RNA deprotection, and methods for RNA purification are known to those of skill in the art.

The siRNA molecules of the present invention can also be synthesized via a tandem synthesis technique, wherein both strands are synthesized as a single continuous oligonucleotide fragment or strand separated by a cleavable linker that is subsequently cleaved to provide separate fragments or strands that hybridize to form the siRNA duplex. The linker can be a polynucleotide linker or a non-nucleotide linker. The tandem synthesis of siRNA can be readily adapted to both multiwell/multiplate synthesis platforms as well as large scale synthesis platforms employing batch reactors, synthesis columns, and the like. Alternatively, siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection. In certain other instances, siRNA molecules can be synthesized as a single continuous oligonucleotide fragment, where the self-complementary sense and antisense regions hybridize to form an siRNA duplex having hairpin secondary structure.

### IV. Target Genes

The nucleic acid that silences expression of a target sequence (e.g., antisense oligonucleotide or siRNA) can be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation, angiogenic genes, immunomodulator genes such as those associated with inflammatory and autoimmune responses, ligand receptor genes, and genes associated with neurodegenerative disorders.

Genes associated with viral infection and survival include those expressed by a virus in order to bind, enter, and replicate in a cell. Of particular interest are viral sequences associated with chronic viral diseases. Viral sequences of particular interest include sequences of Filoviruses such as Ebola virus and Marburg virus (see, e.g., U.S. Patent Publication No. 20070135370; and Geisbert et al., J. Infect. Dis., 193:1650-1657 (2006)); Arenaviruses such as Lassa virus, Junin virus, Machupo virus, Guanarito virus, and Sabia virus (Buchmeier et al., Arenaviridae: the viruses and their replication, In: FIELDS VIROLOGY, Knipe et al. (eds.), 4th ed., Lippincott-Raven, Philadelphia, (2001)); Influenza viruses such as Influenza A, B, and C viruses, (see, e.g., U.S. Provisional Patent Application No. 60/737,945; Steinhauer et al., Annu Rev Genet., 36:305-332 (2002); and Neumann et al., J Gen Virol., 83:2635-2662 (2002)); Hepatitis viruses (Hamasaki et al., FEBS Lett., 543:51 (2003); Yokota et al., EMBO Rep., 4:602 (2003); Schlomai et al., Hepatology, 37:764 (2003); Wilson et al, Proc. Natl. Acad. Sci. USA, 100:2783 (2003); Kapadia et al., Proc. Natl. Acad. Sci. USA, 100:2014 (2003); and FIELDS VIROLOGY, Knipe et al. (eds.), 4th ed., Lippincott-Raven, Philadelphia (2001)); Human Immunodeficiency Virus (HIV) (Banerjea et al., Mol. Ther., 8:62 (2003); Song et al., J. Virol., 77:7174 (2003); Stephenson, JAMA, 289:1494 (2003); Qin et al., Proc. Natl. Acad. Sci. USA, 100:183 (2003)); Herpes viruses (Jia et al., J. Virol., 77:3301 (2003)); and Human Papilloma Viruses (HPV) (Hall et al., J. Virol., 77:6066 (2003); Jiang et al., Oncogene, 21:6041 (2002)).

Exemplary Filovirus nucleic acid sequences that can be silenced include, but are not limited to, nucleic acid sequences encoding structural proteins (e.g., VP30, VP35, nucleoprotein (NP), polymerase protein (L-pol)) and membrane-associated proteins (e.g., VP40, glycoprotein (GP), VP24). Complete genome sequences for Ebola virus are set forth in, e.g., Genbank Accession Nos. NC_{—}002549; AY769362; NC_{—}006432; NC_{—}004161; AY729654; AY354458; AY142960; AB050936; AF522874; AF499101; AF272001; and AF086833. Ebola virus VP24 sequences are set forth in, e.g., Genbank Accession Nos. U77385 and AY058897. Ebola virus L-pol sequences are set forth in, e.g., Genbank Accession No. X67110. Ebola virus VP40 sequences are set forth in, e.g., Genbank Accession No. AY058896. Ebola virus NP sequences are set forth in, e.g., Genbank Accession No. AY058895. Ebola virus GP sequences are set forth in, e.g., Genbank Accession No. AY058898; Sanchez et al., Virus Res., 29:215-240 (1993); Will et al., J. Virol., 67:1203-1210 (1993); Volchkov et al., FEBS Lett., 305:181-184 (1992); and U.S. Pat. No. 6,713,069. Additional Ebola virus sequences are set forth in, e.g., Genbank Accession Nos. L11365 and X61274. Complete genome sequences for Marburg virus are set forth in, e.g., Genbank Accession Nos. NC_{—}001608; AY430365; AY430366; and AY358025. Marburg virus GP sequences are set forth in, e.g., Genbank Accession Nos. AF005734; AF005733; and AF005732. Marburg virus VP35 sequences are set forth in, e.g., Genbank Accession Nos. AF005731 and AF005730. Additional Marburg virus sequences are set forth in, e.g., Genbank Accession Nos. X64406; Z29337; AF005735; and Z12132.

Exemplary Influenza virus nucleic acid sequences that can be silenced include, but are not limited to, nucleic acid sequences encoding nucleoprotein (NP), matrix proteins (M1 and M2), nonstructural proteins (NS1 and NS2), RNA polymerase (PA, PB1, PB2), neuraminidase (NA), and haemagglutinin (HA). Influenza A NP sequences are set forth in, e.g., Genbank Accession Nos. NC_{—}004522; AY818138; AB166863; AB188817; AB189046; AB189054; AB189062; AY646169; AY646177; AY651486; AY651493; AY651494; AY651495; AY651496; AY651497; AY651498; AY651499; AY651500; AY651501; AY651502; AY651503; AY651504; AY651505; AY651506; AY651507; AY651509; AY651528; AY770996; AY790308; AY818138; and AY818140. Influenza A PA sequences are set forth in, e.g., Genbank Accession Nos. AY818132; AY790280; AY646171; AY818132; AY818133; AY646179; AY818134; AY551934; AY651613; AY651610; AY651620; AY651617; AY651600; AY651611; AY651606; AY651618; AY651608; AY651607; AY651605; AY651609; AY651615; AY651616; AY651640; AY651614; AY651612; AY651621; AY651619; AY770995; and AY724786.

Exemplary hepatitis viral nucleic acid sequences that can be silenced include, but are not limited to, nucleic acid sequences involved in transcription and translation (e.g., En1, En2, X, P) and nucleic acid sequences encoding structural proteins (e.g., core proteins including C and C-related proteins, capsid and envelope proteins including S, M, and/or L proteins, or fragments thereof) (see, e.g., FIELDS VIROLOGY, 2001, supra). Exemplary Hepatitis C nucleic acid sequences that can be silenced include, but are not limited to, serine proteases (e.g., NS3/NS4), helicases (e.g. NS3), polymerases (e.g., NS5B), and envelope proteins (e.g., E1, E2, and p7). Hepatitis A nucleic acid sequences are set forth in, e.g., Genbank Accession No. NC_{—}001489; Hepatitis B nucleic acid sequences are set forth in, e.g., Genbank Accession No. NC_{—}003977; Hepatitis C nucleic acid sequences are set forth in, e.g., Genbank Accession No. NC_{—}004102; Hepatitis D nucleic acid sequence are set forth in, e.g., Genbank Accession No. NC_{—}001653; Hepatitis E nucleic acid sequences are set forth in, e.g., Genbank Accession No. NC_{—}001434; and Hepatitis G nucleic acid sequences are set forth in, e.g., Genbank Accession No. NC_{—}001710. Silencing of sequences that encode genes associated with viral infection and survival can conveniently be used in combination with the administration of conventional agents used to treat the viral condition.

Genes associated with metabolic diseases and disorders (e.g., disorders in which the liver is the target and liver diseases and disorders) include, for example, genes expressed in dyslipidemia (e.g., liver X receptors such as LXRα and LXRβ (Genback Accession No. NM_{—}007121), farnesoid X receptors (FXR) (Genbank Accession No. NM_{—}005123), sterol-regulatory element binding protein (SREBP), Site-1 protease (SIP), 3-hydroxy-3-methylglutaryl coenzyme-A reductase (HMG coenzyme-A reductase), Apolipoprotein (ApoB), and Apolipoprotein (ApoE)); and diabetes (e.g., Glucose 6-phosphatase) (see, e.g., Forman et al., Cell, 81:687 (1995); Seol et al., Mol. Endocrinol., 9:72 (1995), Zavacki et al., Proc. Natl. Acad. Sci. USA, 94:7909 (1997); Sakai et al., Cell, 85:1037-1046 (1996); Duncan et al, J. Biol. Chem., 272:12778-12785 (1997); Willy et al., Genes Dev., 9:1033-1045 (1995); Lehmann et al., J. Biol. Chem., 272:3137-3140 (1997); Janowski et al., Nature, 383:728-731 (1996); and Peet et al., Cell, 93:693-704 (1998)). One of skill in the art will appreciate that genes associated with metabolic diseases and disorders (e.g., diseases and disorders in which the liver is a target and liver diseases and disorders) include genes that are expressed in the liver itself as well as and genes expressed in other organs and tissues. Silencing of sequences that encode genes associated with metabolic diseases and disorders can conveniently be used in combination with the administration of conventional agents used to treat the disease or disorder.

Examples of gene sequences associated with tumorigenesis and cell transformation include mitotic kinesins such as Eg5; translocation sequences such as MLL fusion genes, BCR-ABL (Wilda et al., Oncogene, 21:5716 (2002); Scherr et al., Blood, 101:1566 (2003)), TEL-AML1, EWS-FLI1, TLS-FUS, PAX3-FKHR, BCL-2, AML1-ETO, and AML1-MTG8 (Heidenreich et al., Blood, 101:3157 (2003)); overexpressed sequences such as multidrug resistance genes (Nieth et al., FEBS Lett., 545:144 (2003); Wu et al, Cancer Res. 63:1515 (2003)), cyclins (Li et al., Cancer Res., 63:3593 (2003); Zou et al., Genes Dev., 16:2923 (2002)), beta-catenin (Verma et al., Clin Cancer Res., 9:1291 (2003)), telomerase genes (Kosciolek et al., Mol Cancer Ther., 2:209 (2003)), c-MYC, N-MYC, BCL-2, ERBB1, and ERBB2 (Nagy et al. Exp. Cell Res., 285:39 (2003)); and mutated sequences such as RAS (reviewed in Tuschl and Borkhardt, Mol. Interventions, 2:158 (2002)). Silencing of sequences that encode DNA repair enzymes find use in combination with the administration of chemotherapeutic agents (Collis et al., Cancer Res., 63:1550 (2003)). Genes encoding proteins associated with tumor migration are also target sequences of interest, for example, integrins, selectins, and metalloproteinases. The foregoing examples are not exclusive. Any whole or partial gene sequence that facilitates or promotes tumorigenesis or cell transformation, tumor growth, or tumor migration can be included as a template sequence.

Angiogenic genes are able to promote the formation of new vessels. Of particular interest is Vascular Endothelial Growth Factor (VEGF) (Reich et al., Mol. Vis., 9:210 (2003)) or VEGFr. Interfering RNA sequences that target VEGFr are set forth in, e.g., GB 2396864; U.S. Patent Publication No. 20040142895; and CA 2456444.

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see, e.g., U.S. Pat. No. 6,174,861), angiostatin (see, e.g., U.S. Pat. No. 5,639,725), and VEGF-R2 (see, e.g., Decaussin et al., J Pathol., 188: 369-377 (1999)).

Immunomodulator genes are genes that modulate one or more immune responses. Examples of immunomodulator genes include, without limitation, cytokines such as growth factors (e.g., TGF-α, TGF-β, EGF, FGF, IGF, NGF, PDGF, CGF, GM-CSF, SCF, etc.), interleukins (e.g., IL-2, IL-4, IL-12 (Hill et al., J Immunol., 171:691 (2003)), IL-15, IL-18, IL-20, etc.), interferons (e.g., IFN-α, IFN-β, IFN-γ, etc.) and TNF. Fas and Fas Ligand genes are also immunomodulator target sequences of interest (Song et al., Nat. Med., 9:347 (2003)). Genes encoding secondary signaling molecules in hematopoietic and lymphoid cells are also included in the present invention, for example, Tec family kinases such as Bruton's tyrosine kinase (Btk) (Heinonen et al., FEBS Lett., 527:274 (2002)).

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g., inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, glucagon, G-protein coupled receptor ligands, etc. Templates coding for an expansion of trinucleotide repeats (e.g., CAG repeats) find use in silencing pathogenic sequences in neurodegenerative disorders caused by the expansion of trinucleotide repeats, such as spinobulbular muscular atrophy and Huntington's Disease (Caplen et al., Hum. Mol. Genet., 11:175 (2002)).

In addition to their utility in silencing the expression of any of the above-described genes for therapeutic purposes, nucleic acids that silence target gene expression are also useful in research and development applications as well as diagnostic, prophylactic, prognostic, clinical, and other healthcare applications. As a non-limiting example, siRNA molecules can be used in target validation studies directed at testing whether the gene of interest has the potential to be a therapeutic target. siRNA molecules can also be used in target identification studies aimed at discovering genes as potential therapeutic targets.

### V. Carrier Systems

In one aspect, the present invention provides carrier systems containing a modified nucleic acid as described herein, alone or in combination with a nucleic acid that silences expression of a target sequence. In some embodiments, the carrier system is a lipid-based carrier system such as a stabilized nucleic acid-lipid particle (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In additional embodiments, the carrier system is a cyclodextrin-based carrier system such as a cyclodextrin polymer-nucleic acid complex. In further embodiments, the carrier system is a protein-based carrier system such as a cationic peptide-nucleic acid complex. Preferably, the carrier system is a stabilized nucleic acid-lipid particle such as a SNALP or SPLP. One skilled in the art will appreciate that the modified nucleic acid of the present invention can also be delivered as naked molecule.

A. Stabilized Nucleic Acid-Lipid Particles

The stabilized nucleic acid-lipid particles (SNALPs) of the present invention typically comprise a modified nucleic acid as described herein, a cationic lipid, and a non-cationic lipid. In some embodiments, the SNALPs can further comprise a nucleic acid that silences expression of a target sequence. In other embodiments, the SNALPs can further comprise a bilayer stabilizing component (i.e., a conjugated lipid that inhibits aggregation of the particles). The SNALPs may comprise at least 1, 2, 3, 4, 5, 6, 7, 8, 9, 10, or more of the modified nucleic acid molecules described herein, alone or in combination with at least 1, 2, 3, 4, 5, 6, 7, 8, 9, 10, or more nucleic acid molecules that silence expression of a target sequence or a combination of target sequences.

The SNALPs of the present invention typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids are resistant in aqueous solution to degradation with a nuclease when present in the nucleic acid-lipid particles. Nucleic acid-lipid particles and their method of preparation are disclosed in, e.g., U.S. Pat. Nos. 5,753,613; 5,785,992; 5,705,385; 5,976,567; 5,981,501; 6,110,745; and 6,320,017; and PCT Publication No. WO 96/40964.

1. Cationic Lipids

Any of a variety of cationic lipids may be used in the stabilized nucleic acid-lipid particles of the present invention, either alone or in combination with one or more other cationic lipid species or non-cationic lipid species.

Cationic lipids which are useful in the present invention can be any of a number of lipid species which carry a net positive charge at physiological pH. Such lipids include, but are not limited to, DODAC, DODMA, DSDMA, DOTMA, DDAB, DOTAP, DOSPA, DOGS, DC-Chol, DMRIE, and mixtures thereof. A number of these lipids and related analogs have been described in U.S. Patent Publication No. 20060083780; U.S. Pat. Nos. 5,208,036; 5,264,618; 5,279,833; 5,283,185; 5,753,613; and 5,785,992; and PCT Publication No. WO 96/10390. Additionally, a number of commercial preparations of cationic lipids are available and can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA).

Furthermore, cationic lipids of Formula I having the following structures are useful in the present invention

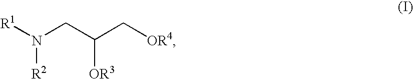

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C18), etc. In certain other instances, R³ and R⁴ are different, i.e., R³ is tetradectrienyl (C14) and R⁴ is linoleyl (C18). In a preferred embodiment, the cationic lipid of Formula I is symmetrical, i.e., R³ and R⁴ are both the same. In another preferred embodiment, both R³ and R⁴ comprise at least two sites of unsaturation. In some embodiments, R³ and R⁴ are independently selected from dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl. In a particularly preferred embodiments, the cationic lipid of Formula I is DLinDMA or DLenDMA.

Moreover, cationic lipids of Formula II having the following structures are useful in the present invention.

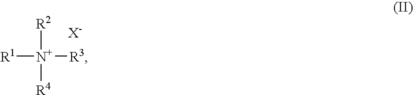

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C18), etc. In certain other instances, R³ and R⁴ are different, i.e., R³ is tetradectrienyl (C14) and R⁴ is linoleyl (C18). In a preferred embodiment, the cationic lipids of the present invention are symmetrical, i.e., R³ and R⁴ are both the same. In another preferred embodiment, both R³ and R⁴ comprise at least two sites of unsaturation. In some embodiments, R³ and R⁴ are independently selected from dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl.

The cationic lipid typically comprises from about 2 mol % to about 60 mol %, from about 5 mol % to about 50 mol %, from about 10 mol % to about 50 mol %, from about 20 mol % to about 50 mol %, from about 20 mol % to about 40 mol %, from about 30 mol % to about 40 mol %, or about 40 mol % of the total lipid present in the particle. It will be readily apparent to one of skill in the art that depending on the intended use of the particles, the proportions of the components can be varied and the delivery efficiency of a particular formulation can be measured using, e.g., an endosomal release parameter (ERP) assay. For example, for systemic delivery, the cationic lipid may comprise from about 5 mol % to about 15 mol % of the total lipid present in the particle, and for local or regional delivery, the cationic lipid may comprise from about 30 mol % to about 50 mol %, or about 40 mol % of the total lipid present in the particle.

2. Non-cationic Lipids

The non-cationic lipids used in the stabilized nucleic acid-lipid particles of the present invention can be any of a variety of neutral uncharged, zwitterionic, or anionic lipids capable of producing a stable complex. They are preferably neutral, although they can alternatively be negatively charged. Examples of non-cationic lipids include, without limitation, phospholipid-related materials such as lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dilinoleoylphosphatidylcholine (DLPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate (DOPE-mal), dipalmitoylphosphatidylethanolamine (DPPE), dimyristoylphosphoethanolamine (DMPE), distearoylphosphatidylethanolamine (DSPE), monomethyl-phosphatidylethanolamine, dimethyl-phosphatidylethanolamine, dielaidoyl-phosphatidylethanolamine (DEPE), and stearoyloleoyl-phosphatidylethanolamine (SOPE). Non-cationic lipids or sterols such as cholesterol may also be present. Additional nonphosphorous containing lipids include, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, ceramide, diacylphosphatidylcholine, and diacylphosphatidylethanolamine. Other lipids such as lysophosphatidylcholine and lysophosphatidylethanolamine may be present. Non-cationic lipids also include polyethylene glycol-based polymers such as PEG 2000, PEG 5000, and polyethylene glycol conjugated to phospholipids or to ceramides (referred to as PEG-Cer), as described in U.S. patent application Ser. No. 08/316,429.

In preferred embodiments, the non-cationic lipid is diacylphosphatidylcholine (e.g., DSPC, DOPC, DPPC, DLPC, POPC), diacylphosphatidylethanolamine (e.g., DOPE, POPE, DPPE, DMPE, DSPE), or a mixture thereof. The acyl groups in these lipids are preferably acyl groups derived from fatty acids having C₁₀-C₂₄ carbon chains. More preferably, the acyl groups are lauroyl, myristoyl, palmitoyl, stearoyl, or oleoyl. In particularly preferred embodiments, the non-cationic lipid will include one or more of cholesterol, DSPC, DPPC, or DSPE.

The non-cationic lipid typically comprises from about 5 mol % to about 90 mol %, from about 10 mol % to about 85 mol %, from about 20 mol % to about 80 mol %, or about 10 mol % of the total lipid present in the particle. The particles may further comprise cholesterol. If present, the cholesterol typically comprises from about 0 mol % to about 10 mol %, from about 2 mol % to about 10 mol %, from about 10 mol % to about 60 mol %, from about 12 mol % to about 58 mol %, from about 20 mol % to about 55 mol %, from about 30 mol % to about 50 mol %, or about 48 mol % of the total lipid present in the particle.

3. Bilayer Stabilizing Component

In addition to cationic and non-cationic lipids, the stabilized nucleic acid-lipid particles of the present invention can comprise a bilayer stabilizing component (BSC) such as an ATTA-lipid or a PEG-lipid, such as PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides, or a mixture thereof (see, e.g., U.S. Pat. No. 5,885,613). In a preferred embodiment, the BSC is a conjugated lipid that prevents the aggregation of particles. Suitable conjugated lipids include, but are not limited to, PEG-lipid conjugates, ATTA-lipid conjugates, cationic-polymer-lipid conjugates (CPLs), and mixtures thereof. In another preferred embodiment, the particles comprise either a PEG-lipid conjugate or an ATTA-lipid conjugate together with a CPL.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). In addition, monomethoxypolyethyleneglycol-acetic acid (MePEG-CH₂COOH) is particularly useful for preparing the PEG-lipid conjugates including, e.g., PEG-DAA conjugates.

In a preferred embodiment, the PEG has an average molecular weight of from about 550 daltons to about 10,000 daltons, more preferably from about 750 daltons to about 5,000 daltons, more preferably from about 1,000 daltons to about 5,000 daltons, more preferably from about 1,500 daltons to about 3,000 daltons, and even more preferably about 2,000 daltons or about 750 daltons. The PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the bilayer stabilizing component. Such phosphatidylethanolamines are commercially available, or can be isolated or synthesized using conventional techniques known to those of skilled in the art. Phosphatidylethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoylphosphatidylethanolamine (DMPE), dipalmitoylphosphatidylethanolamine (DPPE), dioleoylphosphatidylethanolamine (DOPE), and distearoylphosphatidylethanolamine (DSPE).

The term “ATTA” or “polyamide” refers to, without limitation, compounds disclosed in U.S. Pat. Nos. 6,320,017 and 6,586,559. These compounds include a compound having the formula:

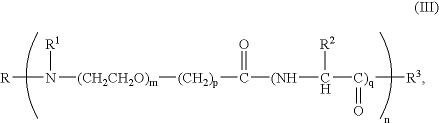

wherein R is a member selected from the group consisting of hydrogen, alkyl and acyl; R¹ is a member selected from the group consisting of hydrogen and alkyl; or optionally, R and R¹ and the nitrogen to which they are bound form an azido moiety; R² is a member of the group selected from hydrogen, optionally substituted alkyl, optionally substituted aryl and a side chain of an amino acid; R³ is a member selected from the group consisting of hydrogen, halogen, hydroxy, alkoxy, mercapto, hydrazino, amino and NR⁴R⁵, wherein R⁴ and R⁵ are independently hydrogen or alkyl; n is 4 to 80; m is 2 to 6; p is 1 to 4; and q is 0 or 1. It will be apparent to those of skill in the art that other polyamides can be used in the compounds of the present invention.

The term “diacylglycerol” refers to a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauryl (C12), myristyl (C14), palmityl (C16), stearyl (C18), and icosyl (C20). In preferred embodiments, R¹ and R² are the same, i.e., R¹ and R² are both myristyl (i.e., dimyristyl), R¹ and R² are both stearyl (i.e., distearyl), etc. Diacylglycerols have the following general formula:

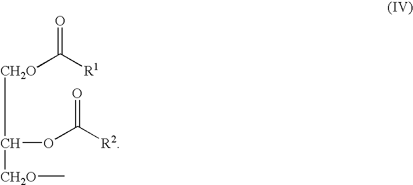

The term “dialkyloxypropyl” refers to a compound having 2 alkyl chains, R¹ and R², both of which have independently between 2 and 30 carbons. The alkyl groups can be saturated or have varying degrees of unsaturation. Dialkyloxypropyls have the following general formula:

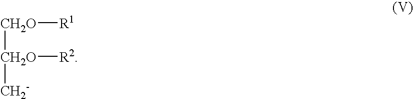

In a preferred embodiment, the PEG-lipid is a PEG-DAA conjugate having the following formula:

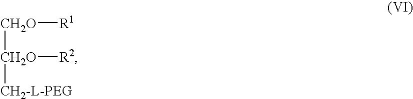

wherein R¹ and R² are independently selected and are long-chain alkyl groups having from about 10 to about 22 carbon atoms; PEG is a polyethyleneglycol; and L is a non-ester containing linker moiety or an ester containing linker moiety as described above. The long-chain alkyl groups can be saturated or unsaturated. Suitable alkyl groups include, but are not limited to, lauryl (C12), myristyl (C14), palmityl (C16), stearyl (C18), and icosyl (C20). In preferred embodiments, R¹ and R² are the same, i.e., R¹ and R² are both myristyl (i.e., dimyristyl), R¹ and R² are both stearyl (i.e., distearyl), etc.

In Formula VI above, the PEG has an average molecular weight ranging from about 550 daltons to about 10,000 daltons, more preferably from about 750 daltons to about 5,000 daltons, more preferably from about 1,000 daltons to about 5,000 daltons, more preferably from about 1,500 daltons to about 3,000 daltons, and even more preferably about 2,000 daltons or about 750 daltons. The PEG can be optionally substituted with alkyl, alkoxy, acyl, or aryl. In a preferred embodiment, the terminal hydroxyl group is substituted with a methoxy or methyl group.

In a preferred embodiment, “L” is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a disulphide linker moiety, a succinamidyl linker moiety, and combinations thereof. In a preferred embodiment, the non-ester containing linker moiety is a carbamate linker moiety (i.e., a PEG-C-DAA conjugate). In another preferred embodiment, the non-ester containing linker moiety is an amido linker moiety (i.e., a PEG-A-DAA conjugate). In yet another preferred embodiment, the non-ester containing linker moiety is a succinamidyl linker moiety (i.e., a PEG-S-DAA conjugate).

The PEG-DAA conjugates are synthesized using standard techniques and reagents known to those of skill in the art. It will be recognized that the PEG-DAA conjugates will contain various amide, amine, ether, thio, carbamate, and urea linkages. Those of skill in the art will recognize that methods and reagents for forming these bonds are well known and readily available. See, e.g., March, ADVANCED ORGANIC CHEMISTRY (Wiley 1992), Larock, COMPREHENSIVE ORGANIC TRANSFORMATIONS (VCH 1989); and Furniss, VOGEL'S TEXTBOOK OF PRACTICAL ORGANIC CHEMISTRY 5th ed. (Longman 1989). It will also be appreciated that any functional groups present may require protection and deprotection at different points in the synthesis of the PEG-DAA conjugates. Those of skill in the art will recognize that such techniques are well known. See, e.g., Green and Wuts, PROTECTIVE GROUPS IN ORGANIC SYNTHESIS (Wiley 1991).

Preferably, the PEG-DAA conjugate is a dilauryloxypropyl (C12)-PEG conjugate, dimyristyloxypropyl (C14)-PEG conjugate, a dipalmityloxypropyl (C16)-PEG conjugate, or a distearyloxypropyl (C18)-PEG conjugate. Those of skill in the art will readily appreciate that other dialkyloxypropyls can be used in the PEG-DAA conjugates of the present invention.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., SNALPs or SPLPs) of the present invention can further comprise cationic poly(ethylene glycol) (PEG) lipids or CPLs that have been designed for insertion into lipid bilayers to impart a positive charge (see, e.g., Chen et al., Bioconj. Chem., 11:433-437 (2000)). Suitable SPLPs and SPLP-CPLs for use in the present invention, and methods of making and using SPLPs and SPLP-CPLs, are disclosed, e.g., in U.S. Pat. No. 6,852,334 and PCT Publication No. WO 00/62813. Cationic polymer lipids (CPLs) useful in the present invention have the following architectural features: (1) a lipid anchor, such as a hydrophobic lipid, for incorporating the CPLs into the lipid bilayer; (2) a hydrophilic spacer, such as a polyethylene glycol, for linking the lipid anchor to a cationic head group; and (3) a polycationic moiety, such as a naturally occurring amino acid, to produce a protonizable cationic head group.

Suitable CPLs include compounds of Formula VII:

A-W—Y (VII),

wherein A, W, and Y are as described below.

With reference to Formula VII, “A” is a lipid moiety such as an amphipathic lipid, a neutral lipid, or a hydrophobic lipid that acts as a lipid anchor. Suitable lipid examples include vesicle-forming lipids or vesicle adopting lipids and include, but are not limited to, diacylglycerolyls, dialkylglycerolyls, N—N-dialkylaminos, 1,2-diacyloxy-3-aminopropanes, and 1,2-dialkyl-3-aminopropanes.

“W” is a polymer or an oligomer such as a hydrophilic polymer or oligomer. Preferably, the hydrophilic polymer is a biocompatible polymer that is nonimmunogenic or possesses low inherent immunogenicity. Alternatively, the hydrophilic polymer can be weakly antigenic if used with appropriate adjuvants. Suitable nonimmunogenic polymers include, but are not limited to, PEG, polyamides, polylactic acid, polyglycolic acid, polylactic acid/polyglycolic acid copolymers, and combinations thereof. In a preferred embodiment, the polymer has a molecular weight of from about 250 to about 7,000 daltons.

“Y” is a polycationic moiety. The term polycationic moiety refers to a compound, derivative, or functional group having a positive charge, preferably at least 2 positive charges at a selected pH, preferably physiological pH. Suitable polycationic moieties include basic amino acids and their derivatives such as arginine, asparagine, glutamine, lysine, and histidine; spermine; spermidine; cationic dendrimers; polyamines; polyamine sugars; and amino polysaccharides. The polycationic moieties can be linear, such as linear tetralysine, branched or dendrimeric in structure. Polycationic moieties have between about 2 to about 15 positive charges, preferably between about 2 to about 12 positive charges, and more preferably between about 2 to about 8 positive charges at selected pH values. The selection of which polycationic moiety to employ may be determined by the type of particle application which is desired.

The charges on the polycationic moieties can be either distributed around the entire particle moiety, or alternatively, they can be a discrete concentration of charge density in one particular area of the particle moiety e.g., a charge spike. If the charge density is distributed on the particle, the charge density can be equally distributed or unequally distributed. All variations of charge distribution of the polycationic moiety are encompassed by the present invention.

The lipid “A” and the nonimmunogenic polymer “W” can be attached by various methods and preferably by covalent attachment. Methods known to those of skill in the art can be used for the covalent attachment of “A” and “W.” Suitable linkages include, but are not limited to, amide, amine, carboxyl, carbonate, carbamate, ester, and hydrazone linkages. It will be apparent to those skilled in the art that “A” and “W” must have complementary functional groups to effectuate the linkage. The reaction of these two groups, one on the lipid and the other on the polymer, will provide the desired linkage. For example, when the lipid is a diacylglycerol and the terminal hydroxyl is activated, for instance with NHS and DCC, to form an active ester, and is then reacted with a polymer which contains an amino group, such as with a polyamide (see, e.g., U.S. Pat. Nos. 6,320,017 and 6,586,559), an amide bond will form between the two groups.

In certain instances, the polycationic moiety can have a ligand attached, such as a targeting ligand or a chelating moiety for complexing calcium. Preferably, after the ligand is attached, the cationic moiety maintains a positive charge. In certain instances, the ligand that is attached has a positive charge. Suitable ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

The bilayer stabilizing component (e.g., PEG-lipid) typically comprises from about 0 mol % to about 20 mol %, from about 0.5 mol % to about 20 mol %, from about 1.5 mol % to about 18 mol %, from about 4 mol % to about 15 mol %, from about 5 mol % to about 12 mol %, or about 2 mol % of the total lipid present in the particle. One of ordinary skill in the art will appreciate that the concentration of the bilayer stabilizing component can be varied depending on the bilayer stabilizing component employed and the rate at which the nucleic acid-lipid particle is to become fusogenic.

By controlling the composition and concentration of the bilayer stabilizing component, one can control the rate at which the bilayer stabilizing component exchanges out of the nucleic acid-lipid particle and, in turn, the rate at which the nucleic acid-lipid particle becomes fusogenic. For instance, when a polyethyleneglycol-phosphatidylethanolamine conjugate or a polyethyleneglycol-ceramide conjugate is used as the bilayer stabilizing component, the rate at which the nucleic acid-lipid particle becomes fusogenic can be varied, for example, by varying the concentration of the bilayer stabilizing component, by varying the molecular weight of the polyethyleneglycol, or by varying the chain length and degree of saturation of the acyl chain groups on the phosphatidylethanolamine or the ceramide. In addition, other variables including, for example, pH, temperature, ionic strength, etc. can be used to vary and/or control the rate at which the nucleic acid-lipid particle becomes fusogenic. Other methods which can be used to control the rate at which the nucleic acid-lipid particle becomes fusogenic will become apparent to those of skill in the art upon reading this disclosure.

B. Additional Carrier Systems

Non-limiting examples of additional lipid-based carrier systems suitable for use in the present invention include lipoplexes (see, e.g., U.S. Patent Publication No. 20030203865; and Zhang et al., J. Control Release, 100:165-180 (2004)), pH-sensitive lipoplexes (see, e.g., U.S. Patent Publication No. 20020192275), reversibly masked lipoplexes (see, e.g., U.S. Patent Publication Nos. 20030180950), cationic lipid-based compositions (see, e.g., U.S. Pat. No. 6,756,054; and U.S. Patent Publication No. 20050234232), cationic liposomes (see, e.g., U.S. Patent Publication Nos. 20030229040, 20020160038, and 20020012998; U.S. Pat. No. 5,908,635; and PCT Publication No. WO 01/72283), anionic liposomes (see, e.g., U.S. Patent Publication No. 20030026831), pH-sensitive liposomes (see, e.g., U.S. Patent Publication No. 20020192274; and AU 2003210303), antibody-coated liposomes (see, e.g., U.S. Patent Publication No. 20030108597; and PCT Publication No. WO 00/50008), cell-type specific liposomes (see, e.g., U.S. Patent Publication No. 20030198664), liposomes containing nucleic acid and peptides (see, e.g., U.S. Pat. No. 6,207,456), liposomes containing lipids derivatized with releasable hydrophilic polymers (see, e.g., U.S. Patent Publication No. 20030031704), lipid-entrapped nucleic acid (see, e.g., PCT Publication Nos. WO 03/057190 and WO 03/059322), lipid-encapsulated nucleic acid (see, e.g., U.S. Patent Publication No. 20030129221; and U.S. Pat. No. 5,756,122), other liposomal compositions (see, e.g., U.S. Patent Publication Nos. 20030035829 and 20030072794; and U.S. Pat. No. 6,200,599), stabilized mixtures of liposomes and emulsions (see, e.g., EP1304160), emulsion compositions (see, e.g., U.S. Pat. No. 6,747,014), and nucleic acid micro-emulsions (see, e.g., U.S. Patent Publication No. 20050037086).

Examples of polymer-based carrier systems suitable for use in the present invention include, but are not limited to, cationic polymer-nucleic acid complexes (i.e., polyplexes). To form a polyplex, a nucleic acid (e.g., a modified nucleic acid as described herein, alone or in combination with a nucleic acid that silences expression of a target sequence) is typically complexed with a cationic polymer having a linear, branched, star, or dendritic polymeric structure that condenses the nucleic acid into positively charged particles capable of interacting with anionic proteoglycans at the cell surface and entering cells by endocytosis. In some embodiments, the polyplex comprises nucleic acid complexed with a cationic polymer such as polyethylenimine (PEI) (see, e.g., U.S. Pat. No. 6,013,240; commercially available from Qbiogene, Inc. (Carlsbad, Calif.) as In vivo jetPEI™, a linear form of PEI), polypropylenimine (PPI), polyvinylpyrrolidone (PVP), poly-L-lysine (PLL), diethylaminoethyl (DEAE)-dextran, poly(O-amino ester) (PAE) polymers (see, e.g., Lynn et al., J. Am. Chem. Soc., 123:8155-8156 (2001)), chitosan, polyamidoamine (PAMAM) dendrimers (see, e.g., Kukowska-Latallo et al., Proc. Natl. Acad. Sci. USA, 93:4897-4902 (1996)), porphyrin (see, e.g., U.S. Pat. No. 6,620,805), polyvinylether (see, e.g., U.S. Patent Publication No. 20040156909), polycyclic amidinium (see, e.g., U.S. Patent Publication No. 20030220289), other polymers comprising primary amine, imine, guanidine, and/or imidazole groups (see, e.g., U.S. Pat. No. 6,013,240; PCT Publication No. WO 96/02655; PCT Publication No. WO 95/21931; Zhang et al., J Control Release, 100:165-180 (2004); and Tiera et al., Curr. Gene Ther., 6:59-71 (2006)), and a mixture thereof. In other embodiments, the polyplex comprises cationic polymer-nucleic acid complexes as described in U.S. Patent Publication Nos. 20060211643, 20050222064, 20030125281, and 20030185890, and PCT Publication No. WO 03/066069; biodegradable poly(β-amino ester) polymer-nucleic acid complexes as described in U.S. Patent Publication No. 20040071654; microparticles containing polymeric matrices as described in U.S. Patent Publication No. 20040142475; other microparticle compositions as described in U.S. Patent Publication No. 20030157030; condensed nucleic acid complexes as described in U.S. Patent Publication No. 20050123600; and nanocapsule and microcapsule compositions as described in AU 2002358514 and PCT Publication No. WO 02/096551.

In certain instances, the modified nucleic acid molecule (alone or in combination with a nucleic acid that silences expression of a target sequence) may be complexed with cyclodextrin or a polymer thereof. Non-limiting examples of cyclodextrin-based carrier systems include the cyclodextrin-modified polymer-nucleic acid complexes described in U.S. Patent Publication No. 20040087024; the linear cyclodextrin copolymer-nucleic acid complexes described in U.S. Pat. Nos. 6,509,323, 6,884,789, and 7,091,192; and the cyclodextrin polymer-complexing agent-nucleic acid complexes described in U.S. Pat. No. 7,018,609. In certain other instances, the modified nucleic acid molecule (alone or in combination with a nucleic acid that silences expression of a target sequence) may be complexed with a peptide or polypeptide. An example of a protein-based carrier system includes, but is not limited to, the cationic oligopeptide-nucleic acid complex described in PCT Publication No. WO 95/21931.

### VI. Preparation of Nucleic Acid-Lipid Particles

The serum-stable nucleic acid-lipid particles of the present invention, in which the nucleic acid molecules described herein are encapsulated in a lipid bilayer and are protected from degradation, can be formed by any method known in the art including, but not limited to, a continuous mixing method, a direct dilution process, a detergent dialysis method, or a modification of a reverse-phase method which utilizes organic solvents to provide a single phase during mixing of the components.

In preferred embodiments, the cationic lipids are lipids of Formula I and II or combinations thereof. In other preferred embodiments, the non-cationic lipids are egg sphingomyelin (ESM), distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), monomethyl-phosphatidylethanolamine, dimethyl-phosphatidylethanolamine, 14:0 PE (1,2-dimyristoyl-phosphatidylethanolamine (DMPE)), 16:0 PE (1,2-dipalmitoyl-phosphatidylethanolamine (DPPE)), 18:0 PE (1,2-distearoyl-phosphatidylethanolamine (DSPE)), 18:1 PE (1,2-dioleoyl-phosphatidylethanolamine (DOPE)), 18:1 trans PE (1,2-dielaidoyl-phosphatidylethanolamine (DEPE)), 18:0-18:1 PE (1-stearoyl-2-oleoyl-phosphatidylethanolamine (SOPE)), 16:0-18:1 PE (1-palmitoyl-2-oleoyl-phosphatidylethanolamine (POPE)), polyethylene glycol-based polymers (e.g., PEG 2000, PEG 5000, PEG-modified diacylglycerols, or PEG-modified dialkyloxypropyls), cholesterol, or combinations thereof. In still other preferred embodiments, the organic solvents are methanol, chloroform, methylene chloride, ethanol, diethyl ether, or combinations thereof.

In a preferred embodiment, the present invention provides for nucleic acid-lipid particles produced via a continuous mixing method, e.g., a process that includes providing an aqueous solution comprising a nucleic acid in a first reservoir, providing an organic lipid solution in a second reservoir, and mixing the aqueous solution with the organic lipid solution such that the organic lipid solution mixes with the aqueous solution so as to substantially instantaneously produce a liposome encapsulating the nucleic acid. This process and the apparatus for carrying this process are described in detail in U.S. Patent Publication No. 20040142025.

The action of continuously introducing lipid and buffer solutions into a mixing environment, such as in a mixing chamber, causes a continuous dilution of the lipid solution with the buffer solution, thereby producing a liposome substantially instantaneously upon mixing. As used herein, the phrase “continuously diluting a lipid solution with a buffer solution” (and variations) generally means that the lipid solution is diluted sufficiently rapidly in a hydration process with sufficient force to effectuate vesicle generation. By mixing the aqueous solution comprising a nucleic acid with the organic lipid solution, the organic lipid solution undergoes a continuous stepwise dilution in the presence of the buffer solution (i.e., aqueous solution) to produce a nucleic acid-lipid particle.

The serum-stable nucleic acid-lipid particles formed using the continuous mixing method typically have a size of from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

In another embodiment, the present invention provides for nucleic acid-lipid particles produced via a direct dilution process that includes forming a liposome solution and immediately and directly introducing the liposome solution into a collection vessel containing a controlled amount of dilution buffer. In preferred aspects, the collection vessel includes one or more elements configured to stir the contents of the collection vessel to facilitate dilution. In one aspect, the amount of dilution buffer present in the collection vessel is substantially equal to the volume of liposome solution introduced thereto. As a non-limiting example, a liposome solution in 45% ethanol when introduced into the collection vessel containing an approximately equal volume of aqueous solution will advantageously yield smaller particles in about 22.5%, about 20%, or about 15% ethanol.

In yet another embodiment, the present invention provides for nucleic acid-lipid particles produced via a direct dilution process in which a third reservoir containing dilution buffer is fluidly coupled to a second mixing region. In this embodiment, the liposome solution formed in a first mixing region is immediately and directly mixed with dilution buffer in the second mixing region. In preferred aspects, the second mixing region includes a T-connector arranged so that the liposome solution and the dilution buffer flows meet as opposing 180° flows; however, connectors providing shallower angles can be used, e.g., from about 27° to about 180°. A pump mechanism delivers a controllable flow of buffer to the second mixing region. In one aspect, the flow rate of dilution buffer provided to the second mixing region is controlled to be substantially equal to the flow rate of liposome solution introduced thereto from the first mixing region. This embodiment advantageously allows for more control of the flow of dilution buffer mixing with the liposome solution in the second mixing region, and therefore also the concentration of liposome solution in buffer throughout the second mixing process. Such control of the dilution buffer flow rate advantageously allows for small particle size formation at reduced concentrations.

These processes and the apparatuses for carrying out these direct dilution processes is described in detail in U.S. patent application Ser. No. 11/495,150.

The serum-stable nucleic acid-lipid particles formed using the direct dilution process typically have a size of from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

In some embodiments, the particles are formed using detergent dialysis. Without intending to be bound by any particular mechanism of formation, a nucleic acid is contacted with a detergent solution of cationic lipids to form a coated nucleic acid complex. These coated nucleic acids can aggregate and precipitate. However, the presence of a detergent reduces this aggregation and allows the coated nucleic acids to react with excess lipids (typically, non-cationic lipids) to form particles in which the nucleic acid is encapsulated in a lipid bilayer. Thus, the serum-stable nucleic acid-lipid particles can be prepared as follows:

(a) combining a nucleic acid with cationic lipids in a detergent solution to form a coated nucleic acid-lipid complex;

(b) contacting non-cationic lipids with the coated nucleic acid-lipid complex to form a detergent solution comprising a nucleic acid-lipid complex and non-cationic lipids; and

(c) dialyzing the detergent solution of step (b) to provide a solution of serum-stable nucleic acid-lipid particles, wherein the nucleic acid is encapsulated in a lipid bilayer and the particles are serum-stable and have a size of from about 50 to about 150 nm.

An initial solution of coated nucleic acid-lipid complexes is formed by combining the nucleic acid with the cationic lipids in a detergent solution. In these embodiments, the detergent solution is preferably an aqueous solution of a neutral detergent having a critical micelle concentration of 15-300 mM, more preferably 20-50 mM. Examples of suitable detergents include, but are not limited to, N,N′-((octanoylimino)-bis-(trimethylene))-bis-(D-gluconamide) (BIGCHAP); BRIJ 35; Deoxy-BIGCHAP; dodecylpoly(ethylene glycol) ether; Tween 20; Tween 40; Tween 60; Tween 80; Tween 85; Mega 8; Mega 9; Zwittergent® 3-08; Zwittergent® 3-10; Triton X-405; hexyl-, heptyl-, octyl- and nonyl-β-D-glucopyranoside; and heptylthioglucopyranoside; with octyl β-D-glucopyranoside and Tween-20 being the most preferred. The concentration of detergent in the detergent solution is typically about 100 mM to about 2 M, preferably from about 200 mM to about 1.5 M.

The cationic lipids and nucleic acids will typically be combined to produce a charge ratio (+/−) of about 1:1 to about 20:1, in a ratio of about 1:1 to about 12:1, or in a ratio of about 2:1 to about 6:1. Additionally, the overall concentration of nucleic acid in solution will typically be from about 25 μg/ml to about 1 mg/ml, from about 25 μg/ml to about 200 μg/ml, or from about 50 μg/ml to about 100 Hg/ml. The combination of nucleic acids and cationic lipids in detergent solution is kept, typically at room temperature, for a period of time which is sufficient for the coated complexes to form. Alternatively, the nucleic acids and cationic lipids can be combined in the detergent solution and warmed to temperatures of up to about 37° C., about 50° C., about 60° C., or about 70° C. For nucleic acids which are particularly sensitive to temperature, the coated complexes can be formed at lower temperatures, typically down to about 4° C.

In some embodiments, the nucleic acid to lipid ratios (mass/mass ratios) in a formed nucleic acid-lipid particle will range from about 0.01 to about 0.2, from about 0.02 to about 0.1, from about 0.03 to about 0.1, or from about 0.01 to about 0.08. The ratio of the starting materials also falls within this range. In other embodiments, the nucleic acid-lipid particle preparation uses about 400 μg nucleic acid per 10 mg total lipid or a nucleic acid to lipid ratio of about 0.01 to about 0.08 and, more preferably, about 0.04, which corresponds to 1.25 mg of total lipid per 50 μg of nucleic acid. In other preferred embodiments, the particle has a nucleic acid:lipid mass ratio of about 0.08.

The detergent solution of the coated nucleic acid-lipid complexes is then contacted with non-cationic lipids to provide a detergent solution of nucleic acid-lipid complexes and non-cationic lipids. The non-cationic lipids which are useful in this step include, diacylphosphatidylcholine, diacylphosphatidylethanolamine, ceramide, sphingomyelin, cephalin, cardiolipin, and cerebrosides. In preferred embodiments, the non-cationic lipids are diacylphosphatidylcholine, diacylphosphatidylethanolamine, ceramide or sphingomyelin. The acyl groups in these lipids are preferably acyl groups derived from fatty acids having C₁₀-C₂₄ carbon chains. More preferably, the acyl groups are lauroyl, myristoyl, palmitoyl, stearoyl or oleoyl. In particularly preferred embodiments, the non-cationic lipids are DSPC, DOPE, POPC, egg phosphatidylcholine (EPC), cholesterol, or a mixture thereof. In the most preferred embodiments, the nucleic acid-lipid particles are fusogenic particles with enhanced properties in vivo and the non-cationic lipid is DSPC or DOPE. In addition, the nucleic acid-lipid particles of the present invention may further comprise cholesterol. In other preferred embodiments, the non-cationic lipids will further comprise polyethylene glycol-based polymers such as PEG 2,000, PEG 5,000, and PEG conjugated to a diacylglycerol, a ceramide, or a phospholipid, as described in, e.g., U.S. Pat. No. 5,820,873 and U.S. Patent Publication No. 20030077829. In further preferred embodiments, the non-cationic lipids will further comprise polyethylene glycol-based polymers such as PEG 2,000, PEG 5,000, and PEG conjugated to a dialkyloxypropyl.

The amount of non-cationic lipid which is used in the present methods is typically about 2 to about 20 mg of total lipids to 50 μg of nucleic acid. Preferably, the amount of total lipid is from about 5 to about 10 mg per 50 μg of nucleic acid.

Following formation of the detergent solution of nucleic acid-lipid complexes and non-cationic lipids, the detergent is removed, preferably by dialysis. The removal of the detergent results in the formation of a lipid-bilayer which surrounds the nucleic acid providing serum-stable nucleic acid-lipid particles which have a size of from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

The serum-stable nucleic acid-lipid particles can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles, is described in U.S. Pat. No. 4,737,323. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and about 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In another group of embodiments, the serum-stable nucleic acid-lipid particles can be prepared as follows:

(a) preparing a mixture comprising cationic lipids and non-cationic lipids in an organic solvent;

(b) contacting an aqueous solution of nucleic acid with the mixture in step (a) to provide a clear single phase; and

(c) removing the organic solvent to provide a suspension of nucleic acid-lipid particles, wherein the nucleic acid is encapsulated in a lipid bilayer and the particles are stable in serum and have a size of from about 50 to about 150 nm.

The nucleic acids, cationic lipids, and non-cationic lipids which are useful in this group of embodiments are as described for the detergent dialysis methods above.

The selection of an organic solvent will typically involve consideration of solvent polarity and the ease with which the solvent can be removed at the later stages of particle formation. The organic solvent, which is also used as a solubilizing agent, is in an amount sufficient to provide a clear single phase mixture of nucleic acid and lipids. Suitable solvents include, but are not limited to, chloroform, dichloromethane, diethylether, cyclohexane, cyclopentane, benzene, toluene, methanol, or other aliphatic alcohols such as propanol, isopropanol, butanol, tert-butanol, iso-butanol, pentanol and hexanol. Combinations of two or more solvents may also be used in the present invention.

Contacting the nucleic acid with the organic solution of cationic and non-cationic lipids is accomplished by mixing together a first solution of nucleic acid, which is typically an aqueous solution, and a second organic solution of the lipids. One of skill in the art will understand that this mixing can take place by any number of methods, for example, by mechanical means such as by using vortex mixers.

After the nucleic acid has been contacted with the organic solution of lipids, the organic solvent is removed, thus forming an aqueous suspension of serum-stable nucleic acid-lipid particles. The methods used to remove the organic solvent will typically involve evaporation at reduced pressures or blowing a stream of inert gas (e.g., nitrogen or argon) across the mixture.

The serum-stable nucleic acid-lipid particles thus formed will typically be sized from about 50 nm to about 150 nm, from about 60 nm to about 130 nm, from about 70 nm to about 110 nm, or from about 70 nm to about 90 nm. To achieve further size reduction or homogeneity of size in the particles, sizing can be conducted as described above.

In other embodiments, the methods will further comprise adding non-lipid polycations which are useful to effect the delivery to cells using the present compositions. Examples of suitable non-lipid polycations include, but are limited to, hexadimethrine bromide (sold under the brand name POLYBRENE®, from Aldrich Chemical Co., Milwaukee, Wis., USA) or other salts of heaxadimethrine. Other suitable polycations include, for example, salts of poly-L-ornithine, poly-L-arginine, poly-L-lysine, poly-D-lysine, polyallylamine, and polyethyleneimine.

In certain embodiments, the formation of the nucleic acid-lipid particles can be carried out either in a mono-phase system (e.g., a Bligh and Dyer monophase or similar mixture of aqueous and organic solvents) or in a two-phase system with suitable mixing.

When formation of the complexes is carried out in a mono-phase system, the cationic lipids and nucleic acids are each dissolved in a volume of the mono-phase mixture. Combination of the two solutions provides a single mixture in which the complexes form. Alternatively, the complexes can form in two-phase mixtures in which the cationic lipids bind to the nucleic acid (which is present in the aqueous phase), and “pull” it into the organic phase.

In another embodiment, the serum-stable nucleic acid-lipid particles can be prepared as follows:

(a) contacting nucleic acids with a solution comprising non-cationic lipids and a detergent to form a nucleic acid-lipid mixture;

(b) contacting cationic lipids with the nucleic acid-lipid mixture to neutralize a portion of the negative charge of the nucleic acids and form a charge-neutralized mixture of nucleic acids and lipids; and

(c) removing the detergent from the charge-neutralized mixture to provide the nucleic acid-lipid particles in which the nucleic acids are protected from degradation.

In one group of embodiments, the solution of non-cationic lipids and detergent is an aqueous solution. Contacting the nucleic acids with the solution of non-cationic lipids and detergent is typically accomplished by mixing together a first solution of nucleic acids and a second solution of the lipids and detergent. One of skill in the art will understand that this mixing can take place by any number of methods, for example, by mechanical means such as by using vortex mixers. Preferably, the nucleic acid solution is also a detergent solution. The amount of non-cationic lipid which is used in the present method is typically determined based on the amount of cationic lipid used, and is typically of from about 0.2 to 5 times the amount of cationic lipid, preferably from about 0.5 to about 2 times the amount of cationic lipid used.

In some embodiments, the nucleic acids are precondensed as described in, e.g., U.S. patent application Ser. No. 09/744,103.

The nucleic acid-lipid mixture thus formed is contacted with cationic lipids to neutralize a portion of the negative charge which is associated with the nucleic acids (or other polyanionic materials) present. The amount of cationic lipids used will typically be sufficient to neutralize at least 50% of the negative charge of the nucleic acid. Preferably, the negative charge will be at least 70% neutralized, more preferably at least 90% neutralized. Cationic lipids which are useful in the present invention include, for example, DLinDMA and DLenDMA. These lipids and related analogs are described in U.S. Patent Publication No. 20060083780.

Contacting the cationic lipids with the nucleic acid-lipid mixture can be accomplished by any of a number of techniques, preferably by mixing together a solution of the cationic lipid and a solution containing the nucleic acid-lipid mixture. Upon mixing the two solutions (or contacting in any other manner), a portion of the negative charge associated with the nucleic acid is neutralized. Nevertheless, the nucleic acid remains in an uncondensed state and acquires hydrophilic characteristics.

After the cationic lipids have been contacted with the nucleic acid-lipid mixture, the detergent (or combination of detergent and organic solvent) is removed, thus forming the nucleic acid-lipid particles. The methods used to remove the detergent will typically involve dialysis. When organic solvents are present, removal is typically accomplished by evaporation at reduced pressures or by blowing a stream of inert gas (e.g., nitrogen or argon) across the mixture.

The particles thus formed will typically be sized from about 50 nm to several microns, about 50 nm to about 150 nm, from about 60 nm to about 130 nm, from about 70 nm to about 110 nm, or from about 70 nm to about 90 nm. To achieve further size reduction or homogeneity of size in the particles, the nucleic acid-lipid particles can be sonicated, filtered, or subjected to other sizing techniques which are used in liposomal formulations and are known to those of skill in the art.

In other embodiments, the methods will further comprise adding non-lipid polycations which are useful to effect the lipofection of cells using the present compositions. Examples of suitable non-lipid polycations include, hexadimethrine bromide (sold under the brandname POLYBRENE®, from Aldrich Chemical Co., Milwaukee, Wis., USA) or other salts of hexadimethrine. Other suitable polycations include, for example, salts of poly-L-ornithine, poly-L-arginine, poly-L-lysine, poly-D-lysine, polyallylamine and polyethyleneimine. Addition of these salts is preferably after the particles have been formed.

In another aspect, the serum-stable nucleic acid-lipid particles can be prepared as follows:

(a) contacting an amount of cationic lipids with nucleic acids in a solution; the solution comprising from about 15-35% water and about 65-85% organic solvent and the amount of cationic lipids being sufficient to produce a +/− charge ratio of from about 0.85 to about 2.0, to provide a hydrophobic nucleic acid-lipid complex;

(b) contacting the hydrophobic, nucleic acid-lipid complex in solution with non-cationic lipids, to provide a nucleic acid-lipid mixture; and

(c) removing the organic solvents from the nucleic acid-lipid mixture to provide nucleic acid-lipid particles in which the nucleic acids are protected from degradation.

The nucleic acids, non-cationic lipids, cationic lipids, and organic solvents which are useful in this aspect of the invention are the same as those described for the methods above which used detergents. In one group of embodiments, the solution of step (a) is a mono-phase. In another group of embodiments, the solution of step (a) is two-phase.

In preferred embodiments, the non-cationic lipids are ESM, DSPC, DOPC, POPC, DPPC, monomethyl-phosphatidylethanolamine, dimethyl-phosphatidylethanolamine, DMPE, DPPE, DSPE, DOPE, DEPE, SOPE, POPE, PEG-based polymers (e.g., PEG 2000, PEG 5000, PEG-modified diacylglycerols, or PEG-modified dialkyloxypropyls), cholesterol, or combinations thereof. In still other preferred embodiments, the organic solvents are methanol, chloroform, methylene chloride, ethanol, diethyl ether, or combinations thereof.

In one embodiment, the nucleic acid is a modified nucleic acid as described herein, alone or in combination with a nucleic acid that silences expression of a target sequence; the cationic lipid is DLindMA, DLenDMA, DODAC, DDAB, DOTMA, DOSPA, DMRIE, DOGS, or combinations thereof, the non-cationic lipid is ESM, DOPE, PEG-DAG, DSPC, DPPC, DPPE, DMPE, monomethyl-phosphatidylethanolamine, dimethyl-phosphatidylethanolamine, DSPE, DEPE, SOPE, POPE, cholesterol, or combinations thereof (e.g., DSPC and PEG-DAA); and the organic solvent is methanol, chloroform, methylene chloride, ethanol, diethyl ether, or combinations thereof.

As above, contacting the nucleic acids with the cationic lipids is typically accomplished by mixing together a first solution of nucleic acids and a second solution of the lipids, preferably by mechanical means such as by using vortex mixers. The resulting mixture contains complexes as described above. These complexes are then converted to particles by the addition of non-cationic lipids and the removal of the organic solvent. The addition of the non-cationic lipids is typically accomplished by simply adding a solution of the non-cationic lipids to the mixture containing the complexes. A reverse addition can also be used. Subsequent removal of organic solvents can be accomplished by methods known to those of skill in the art and also described above.

The amount of non-cationic lipids which is used in this aspect of the invention is typically an amount of from about 0.2 to about 15 times the amount (on a mole basis) of cationic lipids which was used to provide the charge-neutralized nucleic acid-lipid complex. Preferably, the amount is from about 0.5 to about 9 times the amount of cationic lipids used.

In one embodiment, the nucleic acid-lipid particles preparing according to the above-described methods are either net charge neutral or carry an overall charge which provides the particles with greater gene lipofection activity. Preferably, the nucleic acid component of the particles is a nucleic acid which interferes with the production of an undesired protein. In other preferred embodiments, the non-cationic lipid may further comprise cholesterol.

A variety of general methods for making SNALP-CPLs (CPL-containing SNALPs) are discussed herein. Two general techniques include “post-insertion” technique, that is, insertion of a CPL into for example, a pre-formed SNALP, and the “standard” technique,

wherein the CPL is included in the lipid mixture during for example, the SNALP formation steps. The post-insertion technique results in SNALPs having CPLs mainly in the external face of the SNALP bilayer membrane, whereas standard techniques provide SNALPs having CPLs on both internal and external faces. The method is especially useful for vesicles made from phospholipids (which can contain cholesterol) and also for vesicles containing PEG-lipids (such as PEG-DAAs and PEG-DAGs). Methods of making SNALP-CPL, are taught, for example, in U.S. Pat. Nos. 5,705,385; 6,586,410; 5,981,501; 6,534,484; and 6,852,334; U.S. Patent Publication No. 20020072121; and PCT Publication No. WO 00/62813.

### VII. Kits

The present invention also provides nucleic acid-lipid particles in kit form. The kit may comprise a container which is compartmentalized for holding the various elements of the nucleic acid-lipid particles (e.g., the nucleic acids and the individual lipid components of the particles). In some embodiments, the kit may further comprise an endosomal membrane destabilizer (e.g., calcium ions). The kit typically contains the nucleic acid-lipid particle compositions of the present invention, preferably in dehydrated form, with instructions for their rehydration and administration. In certain instances, the particles and/or compositions comprising the particles may have a targeting moiety attached to the surface of the particle. Methods of attaching targeting moieties (e.g., antibodies, proteins) to lipids (such as those used in the present particles) are known to those of skill in the art.

### VIII. Administration of Nucleic Acid-Lipid Particles

Once formed, the serum-stable nucleic acid-lipid particles of the present invention are useful for the introduction of nucleic acids into cells. Accordingly, the present invention also provides methods for introducing one or more nucleic acids into cells. The methods are carried out in vitro or in vivo by first forming the particles as described above and then contacting the particles with the cells for a period of time sufficient for delivery of the one or more nucleic acids to occur.

The nucleic acid-lipid particles of the present invention can be adsorbed to almost any cell type with which they are mixed or contacted. Once adsorbed, the particles can either be endocytosed by a portion of the cells, exchange lipids with cell membranes, or fuse with the cells. Transfer or incorporation of the nucleic acid portion of the particle can take place via any one of these pathways. In particular, when fusion takes place, the particle membrane is integrated into the cell membrane and the contents of the particle combine with the intracellular fluid.

The nucleic acid-lipid particles of the present invention can be administered either alone or in a mixture with a pharmaceutically-acceptable carrier (e.g., physiological saline or phosphate buffer) selected in accordance with the route of administration and standard pharmaceutical practice. Generally, normal buffered saline (e.g., 135-150 mM NaCl) will be employed as the pharmaceutically-acceptable carrier. Other suitable carriers include, e.g., water, buffered water, 0.4% saline, 0.3% glycine, and the like, including glycoproteins for enhanced stability, such as albumin, lipoprotein, globulin, etc. Additional suitable carriers are described in, e.g., REMINGTON'S PHARMACEUTICAL SCIENCES, Mack Publishing Company, Philadelphia, Pa., 17th ed. (1985). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The phrase “pharmaceutically-acceptable” refers to molecular entities and compositions that do not produce an allergic or similar untoward reaction when administered to a human.

The pharmaceutically-acceptable carrier is generally added following particle formation. Thus, after the particle is formed, the particle can be diluted into pharmaceutically-acceptable carriers such as normal buffered saline.

The concentration of particles in the pharmaceutical formulations can vary widely, i.e., from less than about 0.05%, usually at or at least about 2 to 5%, to as much as about 10 to 90% by weight, and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

The pharmaceutical compositions of the present invention may be sterilized by conventional, well-known sterilization techniques. Aqueous solutions can be packaged for use or filtered under aseptic conditions and lyophilized, the lyophilized preparation being combined with a sterile aqueous solution prior to administration. The compositions can contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

A. In Vivo Administration

Systemic delivery for in vivo therapy, i.e., delivery of a therapeutic nucleic acid to a distal target cell via body systems such as the circulation, has been achieved using nucleic acid-lipid particles such as those disclosed in PCT Publication No. WO 96/40964 and U.S. Pat. Nos. 5,705,385; 5,976,567; 5,981,501; and 6,410,328. This latter format provides a fully encapsulated nucleic acid-lipid particle that protects the nucleic acid or combination of nucleic acids from nuclease degradation in serum, is nonimmunogenic, is small in size, and is suitable for repeat dosing.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol, 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). Still other methods of administering lipid-based therapeutics are described in, for example, U.S. Pat. Nos. 3,993,754; 4,145,410; 4,235,871; 4,224,179; 4,522,803; and 4,588,578. The lipid-nucleic acid particles can be administered by direct injection at the site of disease or by injection at a site distal from the site of disease (see, e.g., Culver, HUMAN GENE THERAPY, MaryAnn Liebert, Inc., Publishers, New York. pp. 70-71 (1994)).

The compositions of the present invention, either alone or in combination with other suitable components, can be made into aerosol formulations (i.e., they can be “nebulized”) to be administered via inhalation (e.g., intranasally or intratracheally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally.

Generally, when administered intravenously, the nucleic acid-lipid formulations are formulated with a suitable pharmaceutical carrier. Many pharmaceutically acceptable carriers may be employed in the compositions and methods of the present invention. Suitable formulations for use in the present invention are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, the nucleic acid-lipid particles disclosed herein may be delivered via oral administration to the individual. The particles may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Typically, these oral formulations may contain at least about 0.1% of the nucleic acid-lipid particles or more, although the percentage of the particles may, of course, be varied and may conveniently be between about 1% or 2% and about 60% or 70% or more of the weight or volume of the total formulation. Naturally, the amount of particles in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of the packaged nucleic acid suspended in diluents such as water, saline, or PEG 400; (b) capsules, sachets, or tablets, each containing a predetermined amount of the nucleic acid, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the nucleic acid in a flavor, e.g., sucrose, as well as pastilles comprising the nucleic acid in an inert base, such as gelatin and glycerin or sucrose and acacia emulsions, gels, and the like containing, in addition to the nucleic acid, carriers known in the art.

In another example of their use, nucleic acid-lipid particles can be incorporated into a broad range of topical dosage forms. For instance, the suspension containing the nucleic acid-lipid particles can be formulated and administered as gels, oils, emulsions, topical creams, pastes, ointments, lotions, foams, mousses, and the like.

When preparing pharmaceutical preparations of the nucleic acid-lipid particles of the invention, it is preferable to use quantities of the particles which have been purified to reduce or eliminate empty particles or particles with nucleic acid associated with the external surface.

The methods of the present invention may be practiced in a variety of hosts. Preferred hosts include mammalian species, such as avian (e.g., ducks), primates (e.g., humans and chimpanzees as well as other nonhuman primates), canines, felines, equines, bovines, ovines, caprines, rodents (e.g., rats and mice), lagomorphs, and swine.

The amount of particles administered will depend upon the ratio of nucleic acid to lipid, the particular nucleic acid used, the disease state being diagnosed, the age, weight, and condition of the patient, and the judgment of the clinician, but will generally be between about 0.01 and about 50 mg per kilogram of body weight, preferably between about 0.1 and about 5 mg/kg of body weight, or about 10⁸-10¹⁰ particles per administration (e.g., injection).

B. In Vitro Administration

For in vitro applications, the delivery of nucleic acids can be to any cell grown in culture, whether of plant or animal origin, vertebrate or invertebrate, and of any tissue or type. In preferred embodiments, the cells are animal cells, more preferably mammalian cells, and most preferably human cells.

Contact between the cells and the nucleic acid-lipid particles, when carried out in vitro, takes place in a biologically compatible medium. The concentration of particles varies widely depending on the particular application, but is generally between about 1 μmol and about 10 mmol. Treatment of the cells with the nucleic acid-lipid particles is generally carried out at physiological temperatures (about 37° C.) for periods of time of from about 1 to 48 hours, preferably of from about 2 to 4 hours.

In one group of preferred embodiments, a nucleic acid-lipid particle suspension is added to 60-80% confluent plated cells having a cell density of from about 10³ to about 10⁵ cells/ml, more preferably about 2×10⁴ cells/ml. The concentration of the suspension added to the cells is preferably of from about 0.01 to 0.2 μg/ml, more preferably about 0.1 μg/ml.

Using an Endosomal Release Parameter (ERP) assay, the delivery efficiency of the SNALP or other lipid-based carrier system can be optimized. An ERP assay is described in detail in U.S. Patent Publication No. 20030077829. More particularly, the purpose of an ERP assay is to distinguish the effect of various cationic lipids and helper lipid components of SNALPs based on their relative effect on binding/uptake or fusion with/destabilization of the endosomal membrane. This assay allows one to determine quantitatively how each component of the SNALP or other lipid-based carrier system affects delivery efficiency, thereby optimizing the SNALPs or other lipid-based carrier systems. Usually, an ERP assay measures expression of a reporter protein (e.g., luciferase, β-galactosidase, green fluorescent protein (GFP), etc.), and in some instances, a SNALP formulation optimized for an expression plasmid will also be appropriate for encapsulating the nucleic acids described herein. In other instances, an ERP assay can be adapted to measure downregulation of transcription or translation of a target sequence in the presence or absence of the nucleic acids described herein. By comparing the ERPs for each of the various SNALPs or other lipid-based formulations, one can readily determine the optimized system, e.g., the SNALP or other lipid-based formulation that has the greatest uptake in the cell.

C. Cells for Delivery of Nucleic Acids

The compositions and methods of the present invention can be used to treat a wide variety of cell types, in vivo and in vitro. Suitable cells include, e.g., hematopoietic precursor (stem) cells, fibroblasts, keratinocytes, hepatocytes, endothelial cells, skeletal and smooth muscle cells, osteoblasts, neurons, quiescent lymphocytes, terminally differentiated cells, slow or noncycling primary cells, parenchymal cells, lymphoid cells, epithelial cells, bone cells, and the like.

In vivo delivery of the nucleic acid-lipid particles of the present invention is suited for targeting cells of any cell type. The methods and compositions can be employed with cells of a wide variety of vertebrates, including mammals, such as, e.g., canines, felines, equines, bovines, ovines, caprines, rodents (e.g., mice, rats, and guinea pigs), lagomorphs, swine, and primates (e.g. monkeys, chimpanzees, and humans).

To the extent that tissue culture of cells may be required, it is well-known in the art. For example, Freshney, Culture of Animal Cells, a Manual of Basic Technique, 3rd Ed., Wiley-Liss, New York (1994), Kuchler et al., Biochemical Methods in Cell Culture and Virology, Dowden, Hutchinson and Ross, Inc. (1977), and the references cited therein provide a general guide to the culture of cells. Cultured cell systems often will be in the form of monolayers of cells, although cell suspensions are also used.

D. Detection of SNALPs

In some embodiments, the nucleic acid-lipid particles are detectable in the subject at about 8, 12, 24, 48, 60, 72, or 96 hours, or 6, 8, 10, 12, 14, 16, 18, 19, 22, 24, 25, or 28 days after administration of the particles. The presence of the particles can be detected in the cells, tissues, or other biological samples from the subject. The particles may be detected, e.g., by direct detection of the particles, detection of the modified nucleic acid, detection of the nucleic acid that silences expression of a target sequence, detection of the target sequence of interest (i.e., by detecting expression or reduced expression of the sequence of interest), or a combination thereof.

1. Detection of Particles

Nucleic acid-lipid particles can be detected using any methods known in the art. For example, a label can be coupled directly or indirectly to a component of the SNALP or other carrier system using methods well-known in the art. A wide variety of labels can be used, with the choice of label depending on sensitivity required, ease of conjugation with the SNALP component, stability requirements, and available instrumentation and disposal provisions. Suitable labels include, but are not limited to, spectral labels such as fluorescent dyes (e.g., fluorescein and derivatives, such as fluorescein isothiocyanate (FITC) and Oregon Green™; rhodamine and derivatives such Texas red, tetrarhodimine isothiocynate (TRITC), etc., digoxigenin, biotin, phycoerythrin, AMCA, CyDyes™, and the like; radiolabels such as ³H, ¹²⁵I, ³⁵S, ¹⁴C, ³²P, ³³P, etc.; enzymes such as horse radish peroxidase, alkaline phosphatase, etc.; spectral calorimetric labels such as colloidal gold or colored glass or plastic beads such as polystyrene, polypropylene, latex, etc. The label can be detected using any means known in the art.

2. Detection of Nucleic Acids

Nucleic acids can be detected and quantified herein by any of a number of means well-known to those of skill in the art. The detection of nucleic acids proceeds by well-known methods such as Southern analysis, Northern analysis, gel electrophoresis, PCR, radiolabeling, scintillation counting, and affinity chromatography. Additional analytic biochemical methods such as spectrophotometry, radiography, electrophoresis, capillary electrophoresis, high performance liquid chromatography (HPLC), thin layer chromatography (TLC), and hyperdiffusion chromatography may also be employed.

The selection of a nucleic acid hybridization format is not critical. A variety of nucleic acid hybridization formats are known to those skilled in the art. For example, common formats include sandwich assays and competition or displacement assays. Hybridization techniques are generally described in, e.g., “Nucleic Acid Hybridization, A Practical Approach,” Eds. Hames and Higgins, IRL Press (1985).

The sensitivity of the hybridization assays may be enhanced through use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), Qβ-replicase amplification and other RNA polymerase mediated techniques (e.g., NASBA™) are found in Sambrook et al., In Molecular Cloning. A Laboratory Manual, Cold Spring Harbor Laboratory Press (2000); and Ausubel et al., SHORT PROTOCOLS IN MOLECULAR BIOLOGY, eds., Current Protocols, Greene Publishing Associates, Inc. and John Wiley & Sons, Inc. (2002); as well as U.S. Pat. No. 4,683,202; PCR Protocols, A Guide to Methods and Applications (Innis et al. eds.) Academic Press Inc. San Diego, Calif. (1990); Arnheim & Levinson (Oct. 1, 1990), C&EN 36; The Journal Of NIH Research, 3:81 (1991); Kwoh et al., Proc. Natl. Acad. Sci. USA, 86:1173 (1989); Guatelli et al., Proc. Natl. Acad. Sci. USA, 87:1874 (1990); Lomell et al., J. Clin. Chem., 35:1826 (1989); Landegren et al., Science, 241:1077 (1988); Van Brunt, Biotechnology, 8:291 (1990); Wu and Wallace, Gene, 4:560 (1989); Barringer et al., Gene, 89:117 (1990); and Sooknanan and Malek, Biotechnology, 13:563 (1995). Improved methods of cloning in vitro amplified nucleic acids are described in U.S. Pat. No. 5,426,039. Other methods described in the art are the nucleic acid sequence based amplification (NASBA™, Cangene, Mississauga, Ontario) and Qβ-replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Nucleic acids for use as probes, e.g., in in vitro amplification methods, for use as gene probes, or as inhibitor components are typically synthesized chemically according to the solid phase phosphoramidite triester method described by Beaucage et al., Tetrahedron Letts., 22:1859 1862 (1981), e.g., using an automated synthesizer, as described in Needham VanDevanter et al., Nucleic Acids Res., 12:6159 (1984). Purification of polynucleotides, where necessary, is typically performed by either native acrylamide gel electrophoresis or by anion exchange HPLC as described in Pearson et al., J Chrom., 255:137 149 (1983). The sequence of the synthetic polynucleotides can be verified using the chemical degradation method of Maxam and Gilbert (1980) in Grossman and Moldave (eds.) Academic Press, New York, Methods in Enzymology, 65:499.

An alternative means for determining the level of transcription is in situ hybridization. In situ hybridization assays are well-known and are generally described in Angerer et al., Methods Enzymol., 152:649 (1987). In an in situ hybridization assay, cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

### VIII. Examples

The present invention will be described in greater detail by way of the following examples. The following examples are offered for illustrative purposes, and are not intended to limit the present invention in any manner. Those of skill in the art will readily recognize a variety of noncritical parameters which can be changed or modified to yield essentially the same results.

### Example 1

### Modulation of Immune Response to Toll-Like Receptor Agonists by Modified Single-Stranded RNA

This example illustrates that the immunostimulatory activity of TLR7/8 agonists can be selectively antagonized by single-stranded RNA (ssRNA) having 2′OMe modifications at every uridine residue (“UmodS”).

### Methods

Nucleic acid molecules having the sequences shown in Table 1 were used in this study. The Luc sense strand ssRNA corresponds to nucleotides 1302-1320 of luciferase sequence X84847. The β-gal sense strand ssRNA corresponds to the reverse complement of nucleotides 364853-364871 of E. coli K12 sequence U00096. The GFP sense strand ssRNA corresponds to nucleotides 1801-1819 of green fluorescent protein sequence AY299332.

TABLE 1

| | | |
|---|---|---|
| Luc UmodS | sense | 5′G A mU mU A mU G mU C C G G mU |
| | | mU A mU G mU A U U 3′ |
| β-gal | sense | 5′C U A C A C A A A U C A G C G A |
| | | U U U U U 3′ |
| GFP | sense | 5′G G C U A C G U C C A G G A G C |
| | | G C A U U 3′ |
| polyU21 | | 5′mU mU mU mU mU mU mU mU mU mU mU |
| | | mU mU mU mU mU mU mU mU mU mU 3′ |
| polyU15 | | 5′mU mU mU mU mU mU mU mU mU mU mU |
| | | mU mU mU mU 3′ |
| polyU10 | | 5′mU mU mU mU mU mU mU mU mU mU 3′ |
| polyU5 | | 5′mU mU mU mU mU 3′ |
| RNA40 | | 5′G C C C G U C U G U U G U G U G |
| | | A C U C 3′ |

“in” denotes 2′-O-methyl nucleotides.

All RNA molecules used in this study were heated at 90° C. for 3 min. and then placed at 37° C. for 60 min. Non-denaturing PAGE analysis on a 20% polyacrylamide gel was performed to confirm that ssRNA molecules were intact and that no duplexes had formed upon heating. The RNA molecules were encapsulated in a SNALP formulation comprising PEG-cDMA:DLinDMA:cholesterol:DSPC in a 2:40:48:10 mol % ratio (2:40 SNALP), prepared using a syringe press process.

Fresh human peripheral blood mononuclear cells (PBMCs) were isolated and seeded at about 3×10⁵ cells/well in a total volume of 180 μl. Next, 20 μl of the appropriate SNALP diluted in PBS or 10 μl of naked TLR agonist were added to the PBMCs. For time course experiments, PBMCs were pretreated with UmodS ssRNA or a control and the appropriate naked TLR agonist was added at 0, 0.5, or 2 hours after pretreatment. When the SNALP and naked TLR agonist were added separately, the SNALP was added first followed by the naked TLR agonist. At t=24 hr., the cell supernatant was harvested and IFN-α and/or IL-6 levels were determined using ELISA.

### Results

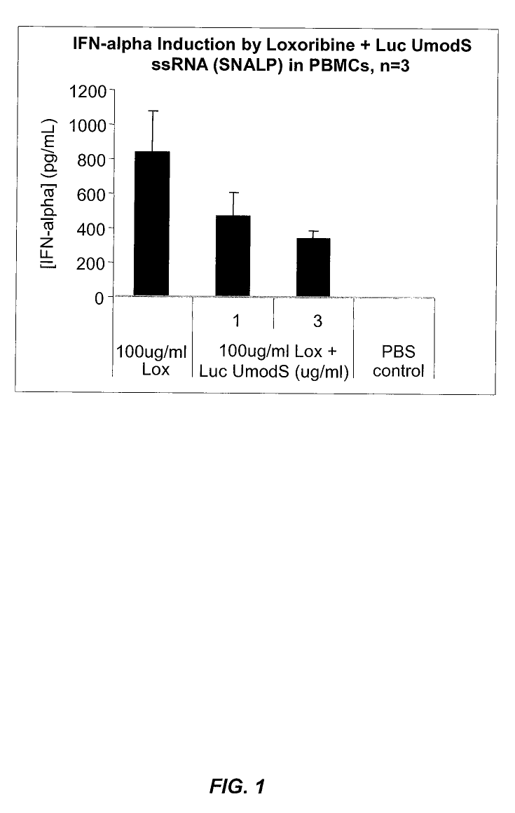

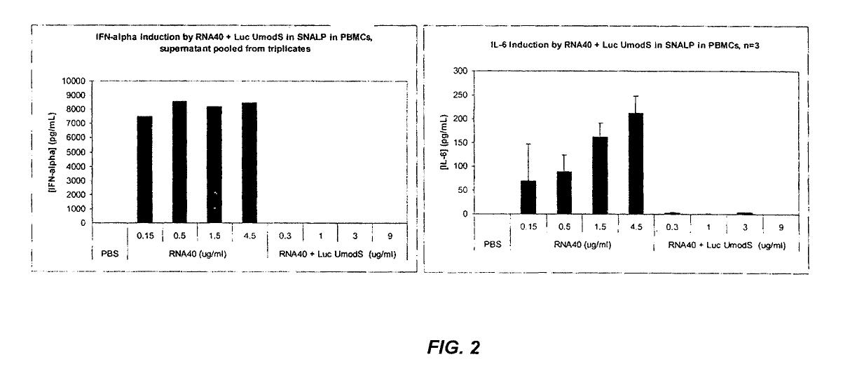

FIG. 1 shows that SNALP-encapsulated Luc UmodS ssRNA can dose-dependently decrease the level of IFN-α induced by naked loxoribine (TLR7 agonist). FIG. 2 shows that Luc UmodS ssRNA can significantly reduce both IFN-α and IL-6 levels induced by the TLR7/8 agonist RNA40 (i.e., a GU rich ssRNA; see, Table 1) when the modified ssRNA and TLR7/8 agonist were co-encapsulated in equimolar amounts in the same SNALP.

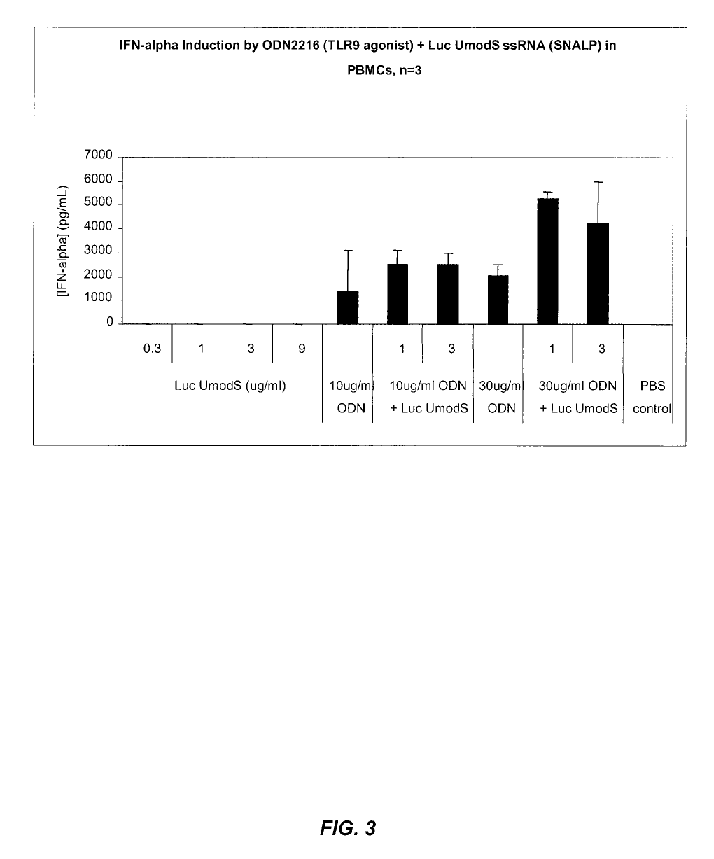

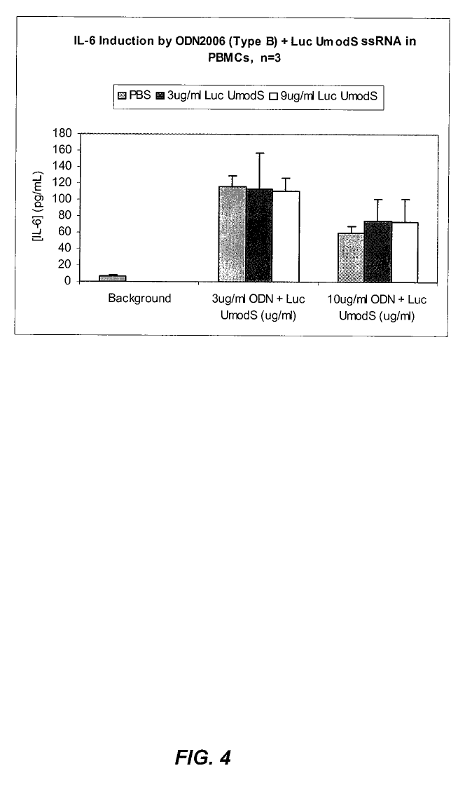

In contrast, FIG. 3 shows that SNALP-encapsulated Luc UmodS ssRNA increased the level of IFN-α induced by naked ODN2216 (TLR9 agonist; a phosphorothioate CpG Type A ODN). FIG. 4 shows that there was no reduction in IL-6 levels induced by naked ODN2006 (TLR9 agonist; a phosphorothioate CpG Type B ODN) when PBMCs were pretreated with SNALP-encapsulated Luc UmodS ssRNA.

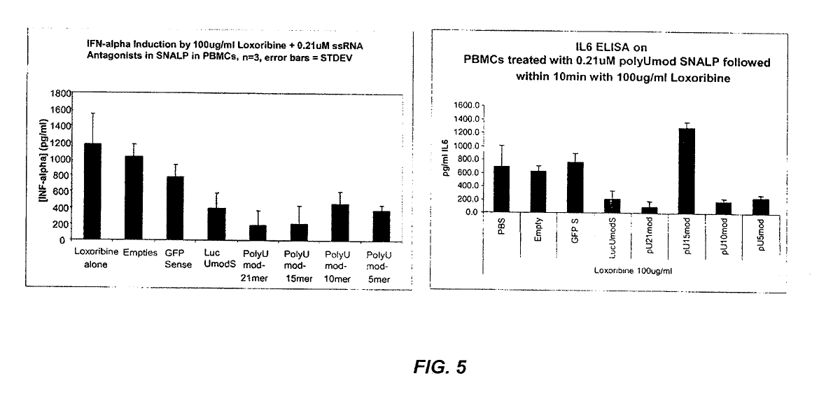

FIG. 5 shows that SNALP-encapsulated Luc UmodS or polyUmod21 ssRNA inhibited both IFN-α and IL-6 levels induced by naked loxoribine (TLR7 agonist), while SNALP-encapsulated polyUmod15 ssRNA inhibited only IFN-α levels.

These data demonstrate that SNALP-encapsulated modified nucleic acid molecules such as UmodS or polyUmod ssRNA can produce a TLR7/8-specific inhibition of cytokine production. As a result, the modified nucleic acid molecules described herein act as specific TLR7/8 antagonists and are particularly useful for the treatment of diseases or disorders associated with TLR7/8 activation such as, for example, autoimmune diseases (e.g., systemic lupus erythematosus, multiple sclerosis, arthritis) and inflammatory diseases.

### Example 2

### Modulation of Immune Response to Immunostimulatory RNA by Modified Single-Stranded RNA

This example illustrates that the immunostimulatory activity of ssRNA (e.g., antisense RNA) or siRNA can be antagonized by non-complementary ssRNA having 2′OMe modifications at every uridine (“UmodS”) residue when the immunostimulatory RNA and modified ssRNA are co-formulated in the same SNALP.

### Methods

Nucleic acid molecules having the sequences shown in Table 2 were used in this study. The NP 1496 sense strand ssRNA corresponds to nucleotides 1498-1516 of influenza nucleocapsid protein (NP) sequence NC_{—}004522. The Luc sense strand ssRNA corresponds to nucleotides 1302-1320 of luciferase sequence X84847. The β-gal sense strand ssRNA corresponds to the reverse complement of nucleotides 364853-364871 of E. coli K12 sequence U00096. The β-gal antisense ssRNA corresponds to 364853-364871 of E. coli K12 sequence U00096. The ApoB antisense ssRNA corresponds to the reverse complement of nucleotides 10165-10187 of human ApoB mRNA sequence NM_{—}000384. The ApoB duplex siRNA sense strand corresponds to nucleotides 10167-10187 of human ApoB mRNA sequence NM_{—}000384. The NP1496 and Luc sense strand ssRNA did not have significant complementarity to the ApoB or β-gal antisense ssRNA.

TABLE 2

| | | |
|---|---|---|
| NP1496 | sense | 5′G G A U C U U A U U U C U U C |
| | | G G A G U U 3′ |
| NP1496 | sense | 5′G G A mU G mU mU A mU mU mU C |
| UmodS | | mU mU G G G A G U U 3′ |
| Luc | sense | 5′G A U U A U G U C C G G U U A |
| | | U G U A U U 3′ |
| Luc | sense | 5′G A mU mU A mU G mU C C G G mU |
| UmodS | | mU A mU G mU A U U 3′ |
| β-gal | sense | 5′G U A C A C A A A U G A G C G A |
| | | A U U U U U 3′ |
| β-gal | sense | 5′G mU A G A G A A A mU C A G C |
| UmodS | | G A mU mU mU U U 3′ |
| ApoB AS | antisense | 5′A U U G G U A U U C A G U G U |
| | | G A U G A C A C 3′ |
| β-gal AS | antisense | 5′A A A A A U C G C U G A U U U |
| | | G U G U A G 3′ |
| ApoB | sense | 5′G U G A U G A G A G U G A A U |
| duplex | | A G G A A U 3′ |
| | antisense | 3′G A G A G U A G U G U G A G U |
| | | U A U G G U U A 5′ |
| polyU21 | | 5′mU mU mU mU mU mU mU mU mU mU |
| | | mU mU mU mU mU mU mU mU mU mU mU |
| | | 3′ |
| polyU15 | | 5′mU mU mU mU mU mU mU mU mU mU |
| | | mU mU mU mU mU 3′ |
| polyU10 | | 5′mU mU mU mU mU mU mU mU mU mU |
| | | 3′ |
| polyU5 | | 5′mU mU mU mU mU 3′ |

“m” denotes 2′-O-methyl nucleotides.

The ssRNA molecules used in this study were heated at 90° C. for 3 min. and then placed at 37° C. for 60 min to disrupt any potential secondary structure. Non-denaturing PAGE analysis on a 20% polyacrylamide gel was performed to confirm that ssRNA molecules were intact and that no duplexes had formed upon heating. siRNA duplexes were prepared by annealing equimolar concentrations of two deprotected and desalted ssRNA oligonucleotides using standard procedures. Formation of annealed siRNA duplexes was confirmed by non-denaturing PAGE analysis on a 20% polyacrylamide gel. The RNA molecules were encapsulated in a 2:40 SNALP formulation prepared using a syringe press process.

Fresh human peripheral blood mononuclear cells (PBMCs) were isolated and seeded at about 3×10⁵ cells/well in a total volume of 180 μl. Next, 20 μl of the appropriate SNALP diluted in PBS were added to the PBMCs. At t=24 hr., the cell supernatant was harvested and IFN-α and/or IL-6 levels were determined using an enzyme-linked immunosorbent assay (ELISA). For example, IFN-α and IL-6 levels can be measured using a sandwich ELISA kit available from PBL Biomedical (Piscataway, N.J.) and eBioscience (San Diego, Calif.), respectively.

### Results

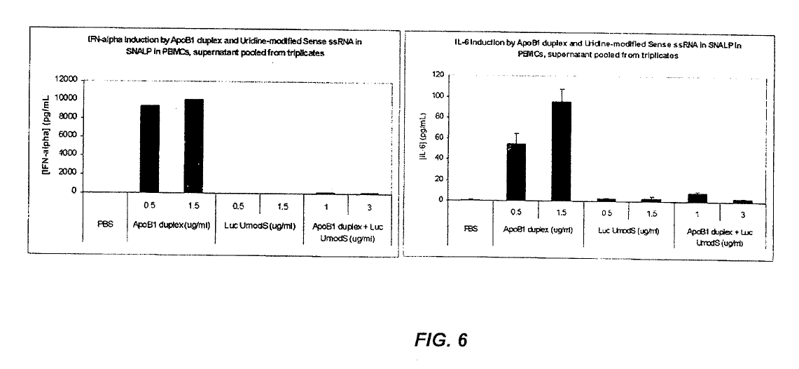

FIG. 6 shows that the immunostimulatory effects of ApoB siRNA were significantly reduced when UmodS ssRNA (e.g., Luc) was co-formulated with the siRNA in the same SNALP.

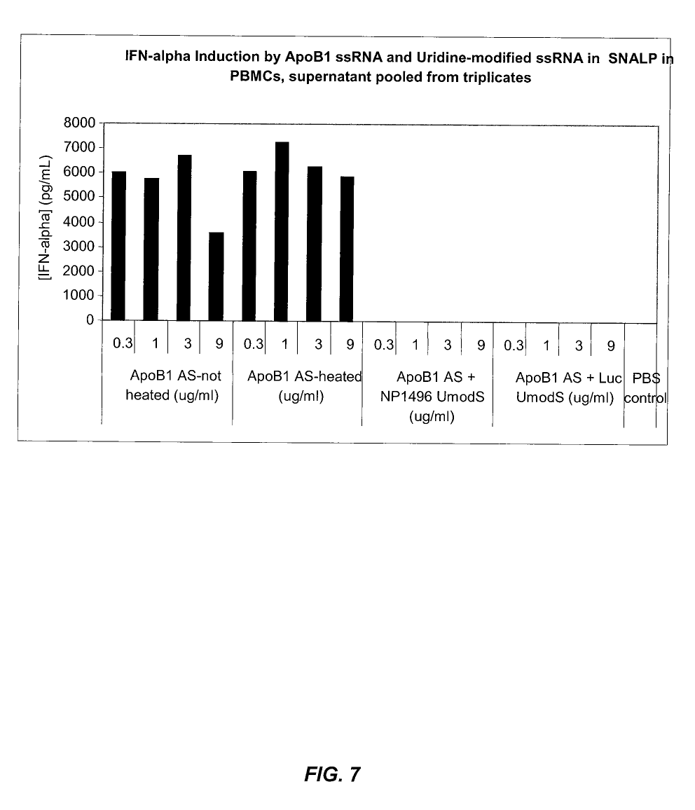

To determine whether temperature affected the immunostimulatory activity of antisense ssRNA, heated and non-heated ApoB antisense ssRNA were tested for their ability to induce IFN-α production. FIG. 7 shows that both heated and non-heated ApoB antisense ssRNA induced similar levels of IFN-α production. Again, the immunostimulatory effects of ApoB antisense ssRNA were abolished when UmodS ssRNA (e.g., NP1496 or Luc) was co-formulated with the antisense ssRNA in the same SNALP. In fact, the ApoB antisense ssRNA induced less than 60 pg/ml IFN-α production in the presence of the modified ssRNA, compared to between 5000-6000 pg/ml in its absence.

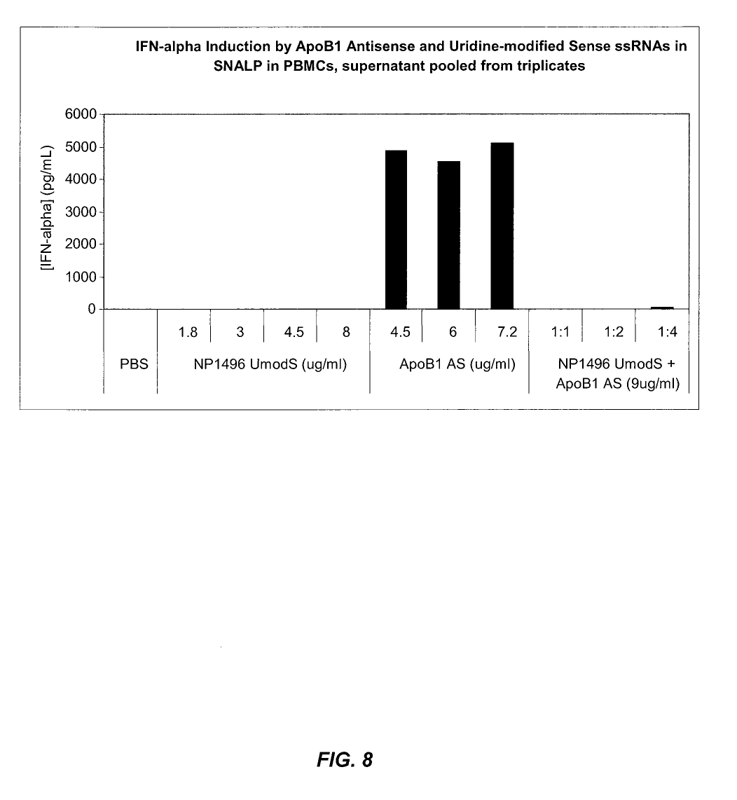

The experiments described above demonstrate that an antagonistic effect was observed when the modified ssRNA (“antagonist”) and the immunostimulatory RNA (“agonist”) were co-encapsulated in the same SNALP (e.g., at a 1:1 molar ratio). FIG. 8 shows that even at 4 times molar excess of antisense ssRNA agonist relative to UmodS ssRNA antagonist, the immunostimulatory effects of the agonist were still abolished by the antagonist when the two nucleic acids were co-formulated in the same SNALP (e.g., about 40 pg/ml of IFN-α induced compared to about 5000 pg/ml). This demonstrates that reduction of the molar amount of the modified ssRNA antagonist to as little as 25% that of the antisense ssRNA agonist (i.e., a molar ratio of 1:4 antagonist:agonist) significantly decreased the agonist-induced immune stimulation. As such, modified ssRNA is very effective at antagonizing the immune response induced by an agonist when the two molecules are co-encapsulated.

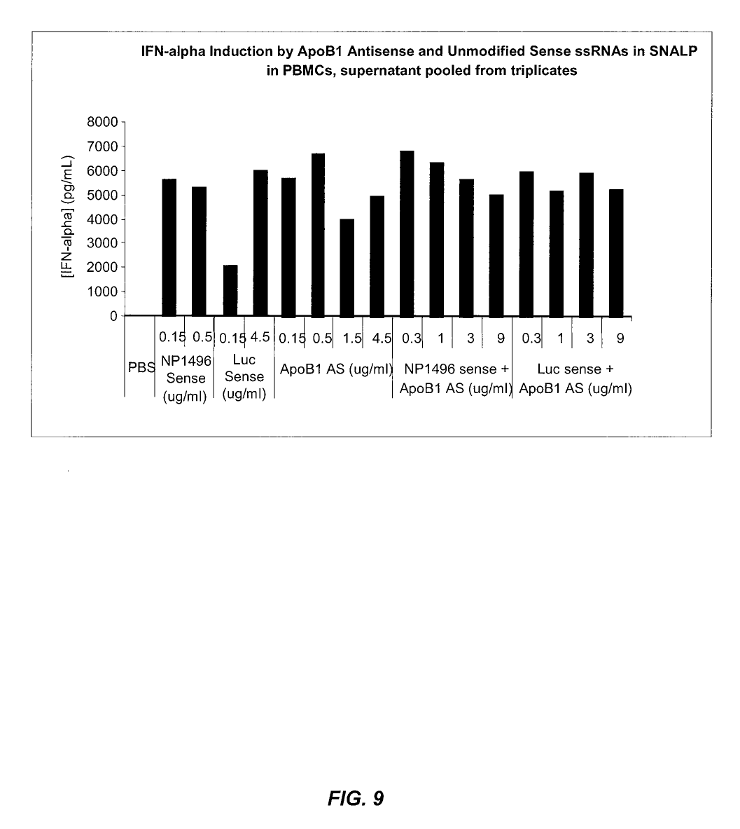

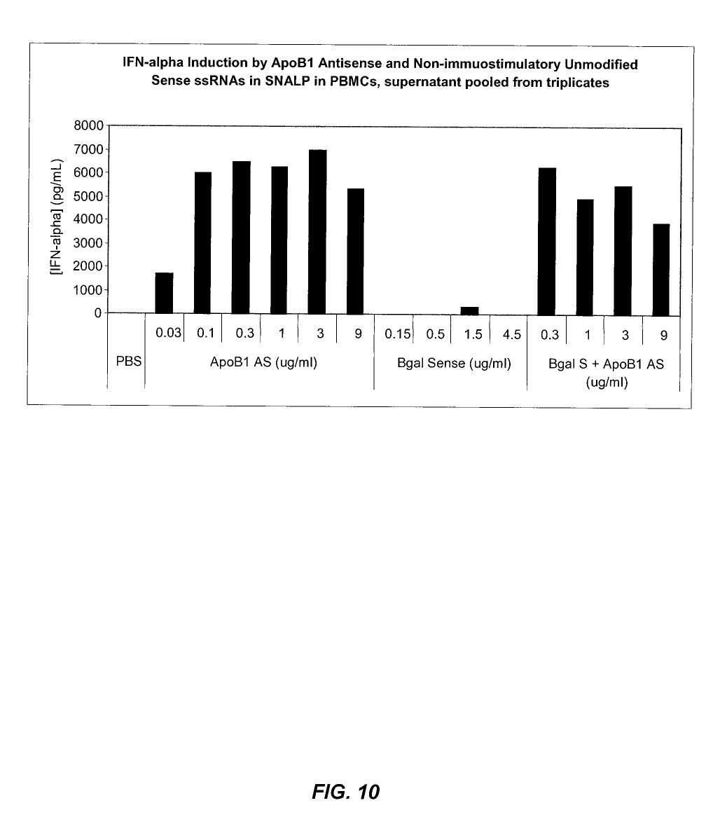

FIGS. 9-10 show that the immunostimulatory effects of ssRNA were antagonized by UmodS ssRNA, but not by unmodified ssRNA, when the two nucleic acid molecules were co-formulated in the same SNALP. In particular, FIG. 9 shows that high levels of IFN-α were still induced when an unmodified NP1496 or Luc sense strand ssRNA and ApoB antisense ssRNA were co-formulated in the same SNALP. Similarly, FIG. 10 shows that high levels of IFN-α were still induced when an unmodified β-gal sense strand ssRNA and ApoB antisense ssRNA were co-formulated in the same SNALP.

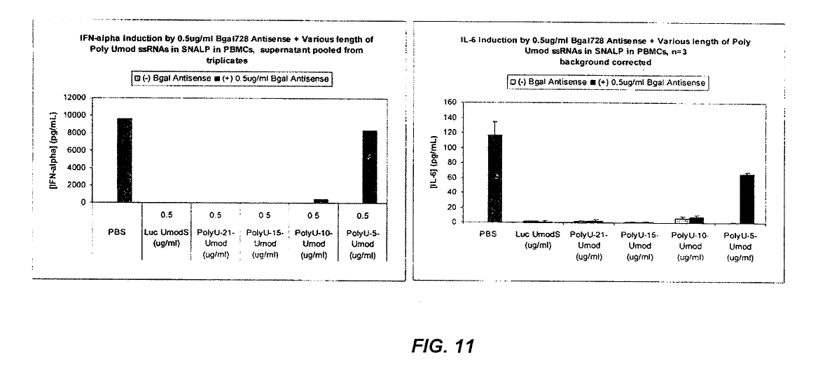

FIG. 11 shows that polyUmod10, polyUmod15, and polyUmod21 ssRNA significantly reduced both IFN-α and IL-6 levels induced by β-gal antisense ssRNA when the modified ssRNA and antisense ssRNA were co-encapsulated in equimolar amounts in the same SNALP. The polyUmod5 ssRNA had less of an antagonistic effect than the polyUmod10, polyUmod15, and polyUmod21 ssRNA.

### Example 3

### 2′-O-Methyl-Modified RNA Molecules Act as TLR7Antagonists

This example illustrates that 2′OMe-modified RNA molecules act as potent inhibitors of RNA-mediated cytokine induction in both human and murine systems. This activity does not require the direct incorporation of 2′OMe nucleotides in the immunostimulatory RNA or that the 2′OMe nucleotide-containing RNA be annealed as a complementary strand to form a duplex. Gene expression analysis of cultured Flt3L-derived dendritic cells (DC) confirmed that 2′OMe-modified RNA blocked the induction of a panel of cytokine and interferon response genes in response to unmodified RNA. These results indicate that 2′OMe-modified RNA molecules act as potent antagonists of immunostimulatory RNA. This example further shows that 2′OMe-modified RNA molecules are able to significantly reduce both IFN-α and IL-6 induction by the small molecule TLR7 agonist loxoribine in human PBMCs, murine Flt3L DCs, and in vivo in mice. These results indicate that 2′OMe-modified RNA molecules find utility as a specific TLR7 inhibitor with potential implications in the treatment of inflammatory and autoimmune diseases that involve TLR7-mediated immune stimulation.

### Materials and Methods

siRNA. siRNA used in these studies were synthesized at The University of Calgary (Alberta, Canada) or at Dharmacon (Lafayette, Colo.) and received as desalted, deprotected oligonucleotides. Duplexes were annealed as described in Judge et al., Mol. Ther., 13:494-505 (2006). All native and 2′OMe-modified RNA oligonucleotide sequences are listed in Table 3. DNA oligonucleotides (ODN) used in these studies were synthesized by IDT (Coralville, Iowa). All ODN sequences are listed in Table 4.

TABLE 3

| | |
|---|---|
| Name | Sequence 5′-3′ |

| | |
|---|---|
| GFP-S | GGCUACGUCCAGGAGCGCAUU |
| βgal-AS | AAAUCGCUGAUUUGUGUAGUU |
| βgal-mU | CmUACACAAAmUCAGCGAmUmUmUUU |
| βgal-mC | mCUAmCAmCAAAUmCAGmCGAUUUUU |
| βgal-mG | CUACACAAAUCAmGCmGAUUUUU |
| βgal-mA | CUmACmACmAmAmAUCmAGCGmAUUUU |
| NP | GGAUCUUAUUUCUUCGGAGUU |
| NP-mU | GGAmUCmUmUAmUmUmUCmUmUCGGAGUU |
| NP-mC | GGAUmCUUAUUUmCUUmCGGAGUU |
| Luc-mU | GAmUmUAmUGmUCCGGmUmUAmUGmUAUU |
| (mU)₂₁ | mUmUmUmUmUmUmUmUmUmUmUmUmUmUmUmUmUmUmUmU |
| (mU)₁₅ | mUmUmUmUmUmUmUmUmUmUmUmUmUmUmU |
| (mU)₁₀ | mUmUmUmUmUmUmUmUmUmU |
| ApoB-S | GUCAUCACACUGAAUACCAAU |
| ApoB-AS | AUUGGUAUUCAGUGUGAUGACAC |

“m” denotes 2′-O-methyl nucleotides.

TABLE 4

| | |
|---|---|
| Name | Sequence 5′-3′ |

| | |
|---|---|
| ODN 2216 (PO) | GGGGGACGATCGTCGGGGGG |
| ODN M362 (PO) | TCGTCGTCGTTCGAACGACGTTGAT |
| ODN 6295 (PO) | TAACGTTGAGGGGCAT |
| ODN 1826 (PO) | TCCATGACGTTCCTGACGTT |

Lipid Encapsulation of RNA and ODN. RNA or ODN were encapsulated in liposomes by a process of spontaneous vesicle formation as described in Judge et al., supra, and Jeffs et al., Pharm. Res., 22:362-372 (2005).

Cell Isolation and Culture. Human PBMCs were isolated from whole blood of healthy donors by a standard Ficoll-Hypaque density centrifugation. Blood was diluted 1:1 with PBS, layered onto Ficoll-Paque Plus, and centrifuged at 1600 RPM for 30 min. PBMCs were washed in PBS twice followed by resuspension in complete media (RPMI 1640, 10% heat inactivated FBS, 2 mM L-glutamine, and 1% penicillin/streptomycin). PBMCs were plated at 2.5×10⁵ cells/well in 96 well plates for cytokine induction assays.

Flt3L-derived dendritic cells (DC) were generated as described in Gilliet et al., J. Exp. Med., 195:953-958 (2002), using 100 ng/ml murine Flt3-ligand (Peprotech) supplemented media. Femurs and tibiae of female Balb/C mice were isolated and rinsed in sterile PBS. The ends of bones were cut and marrow harvested in complete media (RPMI 1640, 10% heat inactivated FBS, 1% penicillin/streptomycin, 2 mM L-glutamine, 1 mM sodium pyruvate, 25 mM HEPES, 50 μM 2-mercaptoethanol). Bone marrow cells were passed through a 70 μm strainer and centrifuged at 1000 rpm for 7 min. and resuspended in complete media supplemented with 100 ng/ml murine Flt3L to 2×10⁶ cells/ml. 2 ml of cells were seeded in 6-well plates and 1 ml fresh media was added every two to three days. On day 9 of culture, non-adherent cells were washed in complete media and plated into 96-well plates at concentrations ranging from 0.5 to 2.5×10⁵ cells/well.

Loxoribine (InvivoGen; San Diego, Calif.), polyIC (Sigma-Aldrich; St. Louis, Mo.), and formulated nucleic acids were diluted in PBS and added to either human PBMC or Flt3L DC cultures. Cells were incubated 24 h at 37° C. before supernatants were assayed for cytokines by ELISA.

Cytokine ELISA. All cytokines were quantified using sandwich ELISA kits according to the manufacturer's instructions. These were mouse and human IFN-α (PBL Biomedical; Piscataway, N.J.) and IL-6 (eBioscience; San Diego, Calif.).

In Vivo Cytokine Induction. Animal studies were performed at Protiva Biotherapeutics in accordance with Canadian Council on Animal Care guidelines and following protocol approval by the local Animal Care and Use Committee. Female Balb/C mice were subjected to a two-week quarantine and acclimation period prior to use at six to eight-weeks of age. RNA formulations were administered by standard injection in the lateral tail vein in 0.2 ml PBS. Six hours after injection, blood was collected by cardiac puncture and processed as plasma for cytokine analysis. In experiments studying inhibition of cytokine induction by loxoribine, formulated RNA (100 μg) was administered IV 2 h prior to IV administration of 1 mg loxoribine in PBS. Blood was collected by cardiac puncture 2 h after administration of loxoribine and processed as plasma for cytokine analysis.

### Results

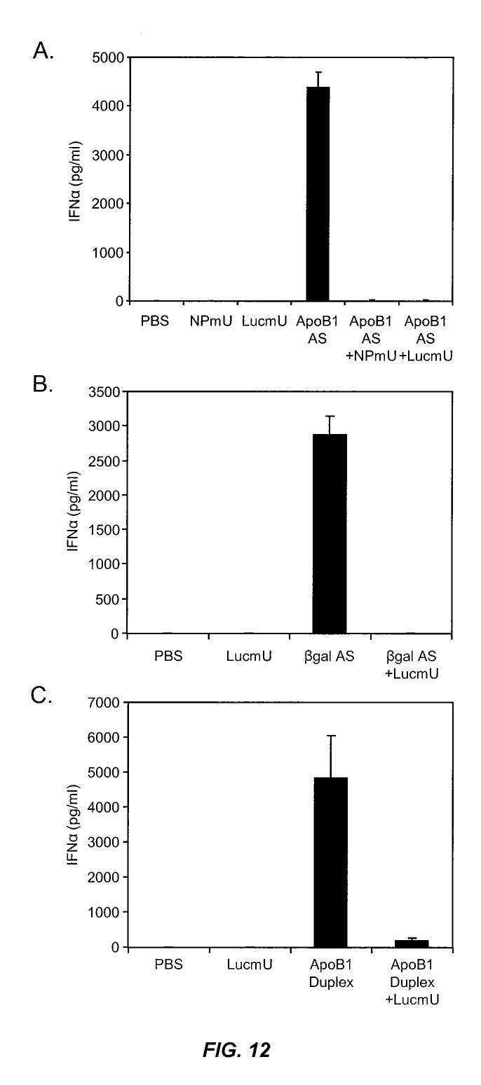

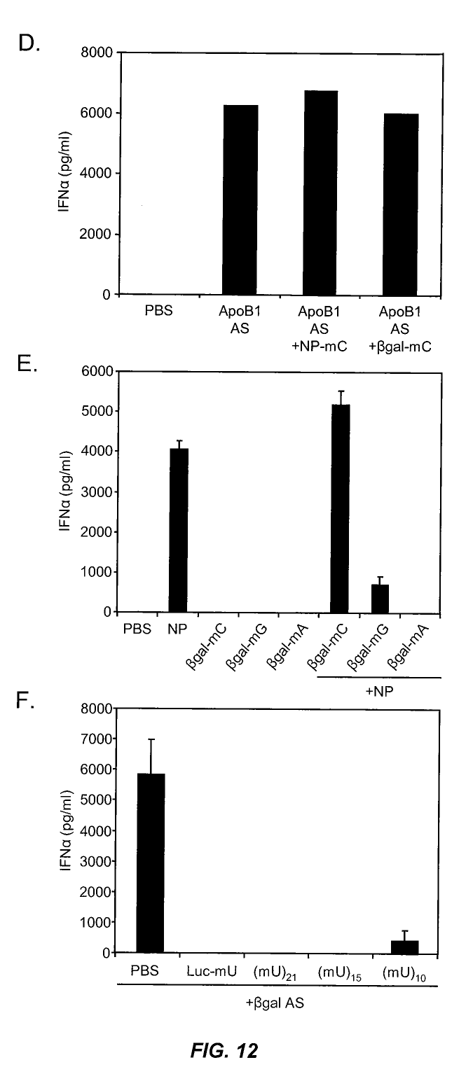

2′OMe-modified ssRNA inhibit cytokine induction by immunostimulatory RNA in both human PBMCs and murine DCs. The introduction of as few as two 2′OMe-uridine or guanosine nucleotides into siRNA duplexes has been shown to effectively abrogate their immunostimulatory activity (Judge et al., Mol. Ther., 13:494-505 (2006)). These inhibitory effects did not require the stimulatory strand within the duplex to be directly modified, indicating that immune recognition of the intact RNA duplex is effectively inhibited by 2′OMe nucleotides incorporated within the molecule. To examine whether this so-called trans-inhibitory effect requires the modified oligonucleotide to be annealed to the immunostimulatory RNA species, non-complementary 2′OMe-modified ssRNA (2′OMe RNA) and immunostimulatory native RNA oligonucleotides were co-encapsulated into lipid particles and tested for their ability to stimulate cytokine responses from human PBMCs. Two ssRNA that contain 2′OMe uridines (Luc-mU and NP-mU) were evaluated (Judge et al., supra). In their modified form, these ssRNA do not induce measurable IFN-α production from human PBMCs (FIG. 12A).

2′OMe RNA was co-encapsulated into lipid nanoparticles at a 1:1 molar ratio with immunostimulatory single-stranded (βgal-AS, ApoB-AS) or duplex (ApoB siRNA) RNA. Lack of duplex formation or dimerization between the 2′OMe RNA and the other RNA species was confirmed by non-denaturing PAGE analysis of the formulated RNA. Each of the immunostimulatory RNA induced high levels of IFN-α when applied to PBMC cultures alone at RNA doses ranging from 0.1 to 3 μg/ml. Strikingly, this immune response was completely abrogated when these native, immunostimulatory RNA were co-administered with either of the non-complementary 2′OMe RNA (FIGS. 12A, B, and C). This inhibitory effect appeared robust since 2′OMe RNA effectively antagonized the IFN-α induction associated with a 4-fold molar excess of the native immunostimulatory RNA. Co-formulation of immunostimulatory RNA with an inherently non-stimulatory native ssRNA had no effect on cytokine induction.

To test whether other 2′OMe nucleotides possessed similar inhibitory capacity, modified RNA were synthesized incorporating either 2′OMe-guanosine, adenosine, or cytidine residues. 2′OMe-G and 2′OMe-A, but not 2′OMe-C modified RNA inhibited cytokine production when co-formulated with immunostimulatory RNA (FIGS. 12D and E).

To determine whether the inhibitory effects of 2′OMe RNA required the modified nucleotides to be presented in a particular sequence or positional context, the inhibitory activity of 2′OMe-uridine homopolymers of 21, 15, or 10 nucleotides in length were tested. 2′OMe-uridine 21mers ((mU)₂₁) and 15mers ((mU)₁₅) were equally as effective at inhibiting cytokine production from human PBMCs when co-formulated with immunostimulatory ssRNA. 2′OMe-uridine 10mers ((mU)₁₀) also significantly reduced cytokine induction, although inhibition with these shorter oligonucleotides was not absolute (FIG. 12F).

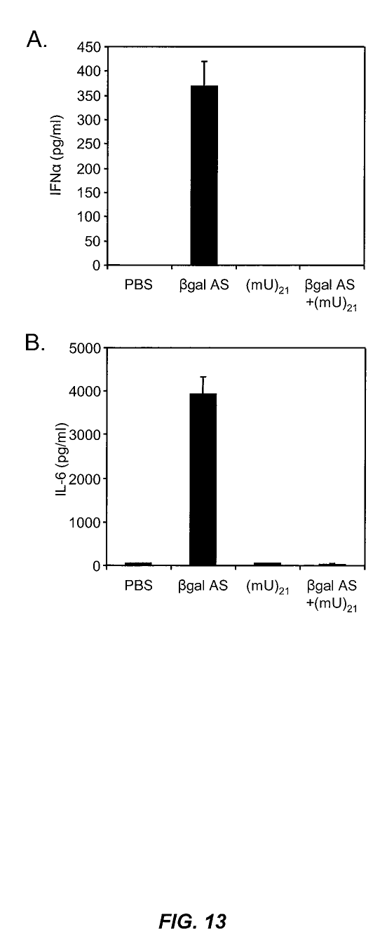

The above experiments conducted in human PBMC cultures were repeated using murine Flt3L-derived dendritic cells (Flt3L DC). Culture of murine bone marrow cells with Flt3L generates a mixed culture of myeloid DC (mDC) and plasmacytoid DC (pDC)-like cells that are responsive to TLR7 ligands including ssRNA (Heil et al., Science, 303:1526-1529 (2004)). The results using murine Flt3L DC were similar to those obtained in human PBMC cultures. Co-administration of (mU)₂₁ with immunostimulatory ssRNA completely abrogated measurable IFN-α and IL-6 production in Flt3L DC (FIG. 13). Taken together, these results demonstrate that 2′OMe RNA potently inhibit immune stimulation mediated by short RNA molecules. Inhibition of this pathway in both mouse and humans is achieved by the incorporation of 2′OMe-U, G, or A nucleotides with no apparent positional or sequence dependent requirements within the modified RNA.

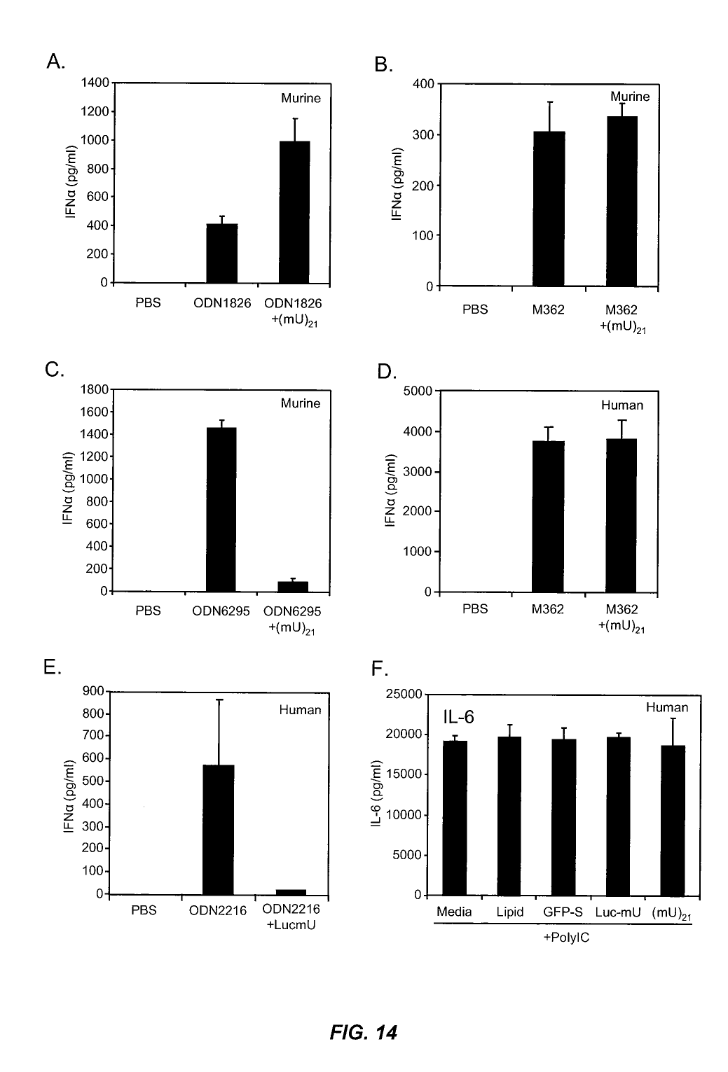

2′OMe RNA does not antagonize cytokine induction by Type B and C CpG ODN. To determine whether 2′OMe RNA also inhibited immune stimulation by TLR9 agonists, various CpG DNA oligonucleotides (ODN) were co-formulated with either (mU)₂₁ RNA or Luc-mU at a 1:1 molar ratio and applied to Flt3L-DC. ODN 6295, 1826, and M362 were selected as representatives of CpG Type A, B, and C ODN, respectively, in the murine system. Each TLR9 agonist was synthesized with phosphodiester backbones and shown to be highly immunostimulatory in murine Flt3L cultures when formulated in lipid nanoparticles. Co-formulation with (mU)₂₁ RNA had no inhibitory effect on the level of IFN-α induction by Type B ODN (1826) or Type C ODN (M362), indicating that 2′OMe RNA does not inherently antagonize TLR9 activation (FIGS. 14A and B). However, co-formulation of 2′OMe RNA was found to cause significant, but not absolute, inhibition of the IFN-α response to a Type A ODN (6295) (FIG. 14C). Experiments were repeated with a range of ODN concentrations from 0.1-5 μg/ml with similar effect.

To further examine this differential effect by 2′OMe RNA on TLR9 agonists, these experiments were repeated in human PBMC cultures. As in the mouse system, co-formulation of Type C ODN (M362) with 2′OMe RNA had little or no effect on IFN-α induction by human PBMCs, whereas the response to a human Type A ODN (2216) was effectively abolished (FIGS. 14D and E). In both mouse and human models, analysis of inflammatory cytokines such as IL-6 and TNF-α mirrored the results for IFN-α. Furthermore, 2′OMe RNA had no effect on the cytokine response elicited by polyI:C (FIG. 14F), a long dsRNA homologue that activates mammalian cells through both TLR3 and PKR. Taken together, these findings indicate that 2′OMe RNA oligonucleotides specifically inhibit TLR7/8-mediated activation by immunostimulatory RNA, but do not directly antagonize the other nucleic acid sensing toll-like receptors.

Although immune stimulation by Type A ODN requires TLR9 (Vollmer et al., Eur. J. Immunol., 34:251-262 (2004)), unlike other ODN, stimulatory activity is dependent on its oligomerization through G-quartets in the ODN sequence. Co-formulation with 2′OMe RNA did not disrupt oligomer formation by the Type A ODN.

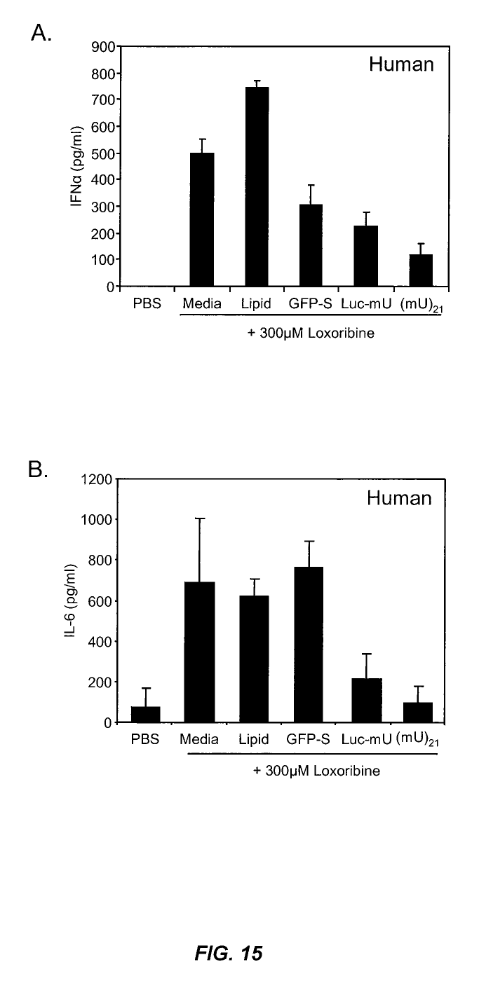

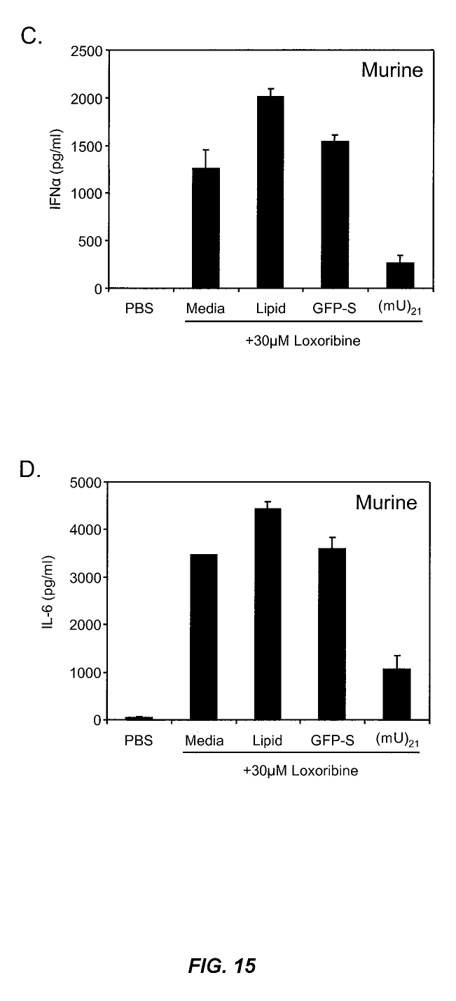

2′OMe RNA inhibit immune activation by the TLR7 agonist loxoribine in vitro. 2′OMe RNA were tested for their ability to inhibit cytokine production by the defined TLR7 agonist loxoribine (7-allyl-8-oxoguanosine, Lox), a guanosine analogue that preferentially activates human and mouse TLR7 (Heil et al., Eur. J. Immunol., 33:2987-2997 (2003)). 300 μM Lox induced robust IFN-α and IL-6 production when added as free drug to human PBMC cultures (FIGS. 15A and B). Co-addition of formulated 2′OMe RNA (Luc-mU) at 1.5 μg/ml (˜0.2 μM) reduced Lox induced IFN-α by 55+/−16% and IL-6 by 62+/−10% in replicate experiments. In addition, (mU)₂₁ provided even more potent inhibition of the response to Lox with IFN-α and IL-6 levels reduced 72+/−5% and 80+/−1%, respectively, based on replicate experiments.

Potent cytokine induction in murine Flt3L DC cultures was achieved with a 10-fold lower concentration of Lox (30 μM) compared to human PBMCs. Under these conditions in which Lox is still in a 140-fold molar excess, 0.2 μM (mU)₂₁ inhibited Lox-mediated IFN-α induction by 87+/−11% and IL-6 by 69+/−1% (FIGS. 15C and D). In both human and murine systems, non-stimulatory native RNA or the lipid vehicle alone had no effect on cytokine induction, indicating that inhibition of the response to Lox was specific to the 2′OMe RNA. These results demonstrate that 2′OMe RNA act as an antagonist to TLR7-mediated immune stimulation.

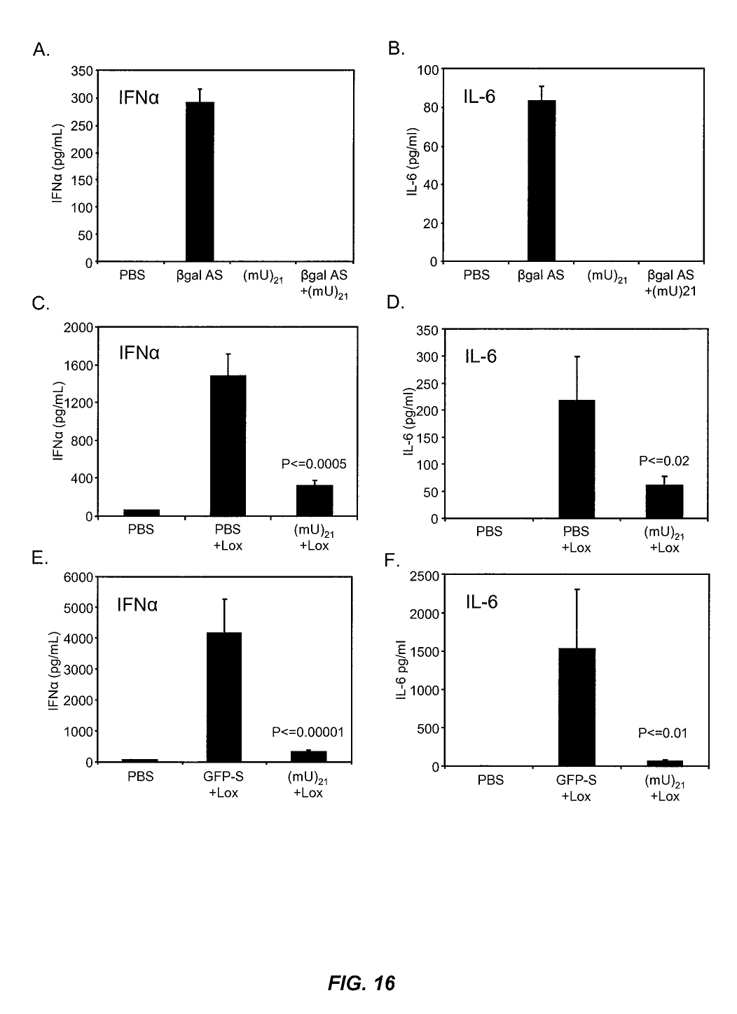

2′OMe RNA inhibit cytokine production by TLR7 agonists in vivo. Mice were treated with immunostimulatory ssRNA (βgal) and 2′OMe-uridine RNA ((mU)₂₁) Co-formulated into lipid particles. Administration of immunostimulatory ssRNA alone induced significant elevations in plasma IFN-α and IL-6, whereas (mU)₂₁ alone induced no measurable cytokine response. As observed in vitro, co-formulation of (mU)₂₁ with the ssRNA agonist eliminated measurable IFN-α and IL-6 induction, indicating that the inhibitory effects of 2′OMe RNA still manifest in vivo (FIGS. 16A and B).

Preliminary studies indicated that plasma cytokine levels peaked around 2 h after IV injection of 1 mg aqueous solution of Lox in mice. To determine if 2′OMe RNA is able to inhibit loxoribine-mediated immune stimulation in vivo, 100 μg of lipid formulated (mU)₂₁, native non-stimulatory ssRNA (GFP-S), or PBS control were administered IV, 2 h prior to treating mice with 1 mg Lox Plasma cytokine levels were then determined 2 h after Lox administration. Control mice pre-treated with non-stimulatory native ssRNA mounted a robust response to loxoribine as assessed by plasma IFN-α and IL-6 levels (FIGS. 16E and F). Pre-treatment with (mU)₂, RNA significantly reduced loxoribine-mediated cytokine induction relative to both the GFP-S ssRNA and the PBS treated mice. Plasma IFN-α and IL-6 levels in (mU)₂₁ treated mice were significantly reduced 79%+/−5% and 72%+/−8%, respectively, compared to PBS pre-treatment (FIGS. 16C and D) or 92%+/−2% and 96%+/−1%, respectively, compared to mice treated with GFP-S ssRNA (FIGS. 16E and F).

Taken together, these results show that 2′OMe RNA act as an antagonist of TLR7-mediated immune stimulation both in vitro and in vivo. This feature of chemically modified RNA may have potential utility in developing novel therapeutics for use in inflammatory and autoimmune diseases that are driven by TLR-7-mediated immune activation (Vollmer et al., J. Exp. Med., 202:1575-1578 (2005); Lau et al., J. Exp. Med., 202:1171-1177 (2005)).

### Discussion

Immune stimulation by short RNA species is effectively blocked by the introduction of 2′OMe nucleotides (Judge et al., Mol. Ther., 13:494-505 (2006); Karikó et al., Immunity, 23:165-175 (2005); Sioud, Eur. J. Immunol., 36:1222-1230 (2006)). To determine how 2′OMe nucleotides may exert this potent inhibitory effect, the ability of 2′OMe RNA oligonucleotides to antagonize TLR7-mediated immune stimulation was tested. This example is the first to detail specific inhibition of TLR7 activation by an antagonistic RNA and illustrates that 2′OMe RNA acts as a potent inhibitor of immune stimulation by short single-stranded and double-stranded RNA molecules in both human and murine systems. This does not require the 2′OMe nucleotides to be directly incorporated into the immunostimulatory RNA or to be annealed as a complementary strand to form a duplex. These observations indicate that 2′OMe-containing RNA act to antagonize the immune recognition of unmodified RNA. The demonstration that 2′OMe RNA also inhibits cytokine induction by the TLR7 ligand loxoribine (Lox) both in vitro and in vivo indicates that 2′OMe RNA acts as a TLR7 antagonist. In sum, this example illustrates that 2′OMe RNA acts to antagonize the immune recognition of unrelated native RNA species as well as small molecule TLR7 ligands such as Lox.

In this example, some experiments utilized a variety of 2′OMe-modified 21mer ssRNA sequences to test for their inhibitory effects on RNA-mediated immune stimulation. Each of these modified RNA proved to be an effective inhibitor, indicating that the antagonistic effect is not sequence-dependent. This is supported by subsequent results demonstrating potent antagonism with 2′OMe-uridine homopolymers as short as 10 nucleotides in length. One exception to these general effects was the observation that RNA containing 2′OMe-cytidines was ineffective at antagonizing RNA-mediated cytokine induction, a finding that is consistent with previous studies that incorporated 2′OMe-cytidines directly into immunostimulatory RNA (Judge et al., supra; Karikó et al., supra). This indicates that the mechanism(s) underlying the immunosuppressive effects of 2′OMe RNA distinguish O-methyl substitutions at the 2′ ribose position in a base-dependent context.

The antagonistic effects of 2′OMe RNA were specific to TLR7 and did not cause global inhibition of other related TLR signaling pathways. 2′OMe RNA inhibited IFN-α and inflammatory cytokine induction by the TLR7 agonist loxoribine in both human and mouse cell culture systems and when administered to mice in vivo. In contrast, 2′OMe RNA had no inhibitory effect on cytokine induction by the TLR9 agonists CpG Type B and C ODN or by the TLR3 agonist polyI:C (FIG. 14). These findings indicate that 2′OMe RNA do not globally disrupt MYD88- or TRIF-dependent pathways utilized by nucleic acid-sensing TLR's. TLR8 is phylogenetically close to TLR7 and is also activated by ssRNA in humans (Heil et al., Science, 303:1526-1529 (2004)). The expression patterns, however, are distinct, with B cells and pDC typically expressing TLR7, while myeloid DC and monocytes constitutively express TLR8. These differences likely account for the respective bias towards predominantly IFN-α (TLR7) or pro-inflammatory cytokine (TLR8) induction profiles (Gorden, et al., J. Immunol., 174:1259-1268 (2005)). Since 2′OMe RNA abolishes both these cytokine responses to ssRNA in human PBMC that contain TLR7 and TLR8 expressing cell types, these findings indicate that 2′OMe RNA can antagonize both TLR7 and TLR8 in human cells.

Given that 2′OMe RNA did not inhibit responses towards CpG Type B and Type C ODN, it was surprising to find that these modified RNA were able to antagonize human and mouse CpG Type A ODN (FIGS. 14C and E). This indicates that ssRNA TLR7/8 agonists and Type A ODN may share a common receptor or adaptor in their signaling pathways that is inhibited by 2′OMe RNA. It has been shown that neither of these classes of TLR agonists induce strong activation of NF-κB reporter constructs in TLR-expressing HEK293 cells, indicating that these TLR ligands may require an adaptor or co-receptor that is absent from the TLR-HEK293 cell system (Judge et al., Nature Biotech., 23:457-462 (2005); Vollmer et al., Eur. J. Immunol., 34:251-262 (2004)).

Dysregulated activation of the immune system through TLR pathways is believed to drive many inflammatory and autoimmune disorders. TLR7 has recently been shown to play a major role in the activation of autoreactive B cells (Vollmer et al., J. Exp. Med., 202:1575-1585 (2005); Lau et al., J. Exp. Med., 202:1171-1177 (2005)) and subsequent development of systemic autoimmune disease such as systemic lupus erythematosus (SLE) (Pisitkun et al., Science, 312:1669-1672 (2006); Subramanian et al., Proc. Natl. Acad. Sci. USA, 103:9970-9975 (2006); Christensen et al., Immunity, 25:417-428 (2006)). The production of both pathogenic autoantibodies and Type I interferons that are hallmarks of SLE pathogenesis (Pascual et al., Curr. Opin. Immunol., 18:676-682 (2006)) can be driven by RNA associated autoantigens and immune complexes through TLR7 activation (Vollmer et al., supra; Lau et al., supra; Savarese et al., Blood, 107:3229-3234 (2006)). Antagonism of the TLR7 pathway therefore provides a potential therapeutic option that targets several key components of this disease. The results described herein indicate that 2′OMe RNA may represent a novel therapeutic candidate for this application.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents, PCT publications, and Genbank Accession Nos., are incorporated herein by reference for all purposes.

What is claimed is:

1. A composition comprising a nucleic acid having at least one modified nucleotide and a nucleic acid that silences expression of a target sequence.

2. The composition of claim 1, wherein the nucleic acid having at least one modified nucleotide comprises a single-stranded RNA (ssRNA).

3. The composition of claim 1, wherein the nucleic acid having at least one modified nucleotide comprises at least one 2′-O-methyl (2′OMe) nucleotide.

4. The composition of claim 1, wherein the nucleic acid that silences expression of the target sequence comprises an antisense oligonucleotide or siRNA.

5. The composition of claim 1, wherein the nucleic acid having at least one modified nucleotide does not have complementarity to the nucleic acid that silences expression of the target sequence.

6. The composition of claim 1, wherein the nucleic acid that silences expression of the target sequence comprises unmodified nucleotides.

7. The composition of claim 1, wherein the nucleic acid that silences expression of the target sequence comprises at least one modified nucleotide.

8. The composition of claim 1, wherein the nucleic acid that silences expression of the target sequence has immunostimulatory activity.

9. The composition of claim 8, wherein the nucleic acid having at least one modified nucleotide reduces the immunostimulatory activity of the nucleic acid that silences expression of the target sequence.

10. The composition of claim 1, wherein the nucleic acid having at least one modified nucleotide modulates Toll-like receptor activation.

11. The composition of claim 10, wherein the Toll-like receptor is selected from the group consisting of TLR7, TLR8, and a combination thereof.

12. The composition of claim 1, further comprising a pharmaceutically acceptable carrier.

13. A nucleic acid-lipid particle comprising:
a composition of claim 1;
a cationic lipid; and
a non-cationic lipid.

14. The nucleic acid-lipid particle of claim 13, wherein the cationic lipid is selected from the group consisting of 1,2-DiLinoleyloxy-N,N-dimethylaminopropane (DLinDMA), 1,2-Dilinolenyloxy-N,N-dimethylaminopropane (DLenDMA), and a mixture thereof.

15. The nucleic acid-lipid particle of claim 14, wherein the non-cationic lipid is selected from the group consisting of distearoylphosphatidylcholine (DSPC), dipalmitoylphosphatidylcholine (DPPC), distearoylphosphatidylethanolamine (DSPE), and a mixture thereof.

16. The nucleic acid-lipid particle of claim 15, further comprising cholesterol.

17. The nucleic acid-lipid particle of claim 16, further comprising a polyethyleneglycol (PEG)-lipid conjugate.

18. The nucleic acid-lipid particle of claim 17, wherein the PEG-lipid conjugate comprises a PEG-dialkyloxypropyl (DAA) conjugate.

19. The nucleic acid-lipid particle of claim 18, wherein the PEG-DAA conjugate comprises a PEG-dimyristyloxypropyl (C₁₄).

20. The nucleic acid-lipid particle of claim 18, wherein the cationic lipid comprises about 40 mol % of the total lipid present in the particle.

21. The nucleic acid-lipid particle of claim 20, wherein the non-cationic lipid comprises about 10 mol % of the total lipid present in the particle.

22. The nucleic acid-lipid particle of claim 21, wherein the cholesterol comprises about 48 mol % of the total lipid present in the particle.

23. The nucleic acid-lipid particle of claim 22, wherein the PEG-DAA conjugate comprises about 2 mol % of the total lipid present in the particle.

24. The nucleic acid-lipid particle of claim 13, wherein the nucleic acid having at least one modified nucleotide and the nucleic acid that silences expression of the target sequence are co-encapsulated in the same nucleic acid-lipid particle.

25. A pharmaceutical composition comprising a nucleic acid-lipid particle of claim 13 and a pharmaceutically acceptable carrier.

26. A method for silencing expression of a target sequence, the method comprising administering to a mammalian subject an effective amount of a composition of claim 1.

27. The method of claim 26, wherein the mammalian subject is a human.

28. The method of claim 26, wherein the composition is in a nucleic acid-lipid particle comprising:
the nucleic acid having at least one modified nucleotide;
the nucleic acid that silences expression of the target sequence;
a cationic lipid; and
a non-cationic lipid.

29. The method of claim 28, wherein the nucleic acid-lipid particle further comprises a PEG-lipid conjugate.

30. The method of claim 28, wherein the nucleic acid having at least one modified nucleotide and the nucleic acid that silences expression of the target sequence are co-encapsulated in the same nucleic acid-lipid particle.

31. A method for modulating Toll-like receptor activation, the method comprising administering to a mammalian subject an effective amount of a nucleic acid having at least one 2′OMe nucleotide.

32. The method of claim 31, wherein the mammalian subject is a human.

33. The method of claim 31, wherein the nucleic acid having at least one 2′OMe nucleotide comprises a single-stranded RNA (ssRNA).

34. The method of claim 31, wherein the nucleic acid having at least one 2′OMe nucleotide comprises a sequence of about 5 to about 60 nucleotides in length.

35. The method of claim 31, wherein the Toll-like receptor is selected from the group consisting of TLR7, TLR8, and a combination thereof.

36. The method of claim 31, wherein the nucleic acid having at least one 2′OMe nucleotide is in a nucleic acid-lipid particle comprising:
the nucleic acid having at least one 2′OMe nucleotide;
a cationic lipid; and
a non-cationic lipid.

37. The method of claim 36, wherein the nucleic acid-lipid particle further comprises a PEG-lipid conjugate.

38. The method of claim 31, wherein the nucleic acid having at least one 2′OMe nucleotide is administered for the treatment of a disease or disorder associated with Toll-like receptor activation.

39. The method of claim 38, wherein the disease or disorder associated with Toll-like receptor activation is an autoimmune disease or inflammatory disease.

40. The method of claim 39, wherein the autoimmune disease is systemic lupus erythematosus (SLE), multiple sclerosis, or arthritis.
